(19) United States
(12) Patent Application Publication
(10) Pub. No.: US 2024/0041590 A1
(43) Pub. Date: Feb. 8, 2024

(54) IMPLANTABLE CORTICOSTEROID MATRIX FOR SINUS CONDITION

(71) Applicant: LYRA THERAPEUTICS, INC., Watertown, MA (US)

(72) Inventors: Anders Cervin, Watertown, MA (US); Joanne Rimmer, Watertown, MA (US); Agnieszka Wrobel, Watertown, MA (US); Yogen Abelak, Watertown, MA (US); Lindsay Brayton, Watertown, MA (US); Yina Kuang, Watertown, MA (US)

(21) Appl. No.: 18/235,449

(22) Filed: Aug. 18, 2023

Related U.S. Application Data
(63) Continuation of application No. PCT/US22/19379, filed on Mar. 8, 2022.
(60) Provisional application No. 63/271,972, filed on Oct. 26, 2021.
(60) Provisional application No. 63/251,159, filed on Oct. 1, 2021.
(60) Provisional application No. 63/241,224, filed on Sep. 7, 2021.
(60) Provisional application No. 63/181,594, filed on Apr. 29, 2021.
(60) Provisional application No. 63/158,017, filed on Mar. 8, 2021.

(51) Int. Cl. A61F 2/18; A61K 9/00

(52) U.S. Cl. CPC A61F 2/186; A61K 9/0043; A61F 2250/0067

(57) ABSTRACT

The present invention relates to materials, devices, kits and methods for use in treatment of patients having a sinus condition, including those with (or at risk of having) severe chronic rhinosinusitis (CRS) symptoms. More specifically, in some embodiments, a device is a long-acting corticosteroid matrix.

### CROSS-REFERENCE TO RELATED APPLICATIONS

This application is a Continuation of, and claims priority to, pending PCT/US22/19379 filed Mar. 8, 2022, which claims benefit of Provisional Application Ser. Nos. 63/271,972 filed now expired; Provisional Application Ser. No. 63/251,159 filed Oct. 1, 2021, now expired; Provisional Application Ser. No. 63/241,224 filed Sep. 7, 2021, now expired; Provisional Application Ser. No. 63/181,594 filed Apr. 29, 2021, now expired; and Provisional Application Ser. No. 63/158,017 filed on Mar. 8, 2021, now expired, the contents of which are incorporated herein in their entirety.

### FIELD OF THE INVENTION

The present invention relates to materials, devices, kits and methods for use in treatment of patients having a sinus condition, including those with (or at risk of having) severe chronic rhinosinusitis (CRS) symptoms. More specifically, in some embodiments, a device is a long-acting corticosteroid matrix.

### BACKGROUND OF THE INVENTION

Topical steroids are first-line treatment for chronic rhinosinusitis (CRS), but fail to provide adequate symptom control for many patients. Yet, current clinical practice guidelines universally recommend topical corticosteroids as a first-line treatment for CRS patients.^{2, 3} Corticosteroids reduce the inflammation associated with known CRS endotypes and are particularly effective at suppressing T2 (T cell type II) inflammation.

However, intranasal corticosteroids (INCS) do not provide adequate symptom control for patients due to one or more of their limited ability to reach inflammation deep within the sinonasal passages, rapid clearance rates, and poor patient compliance.^{7, 8} Approximately 40-60% of CRS patients fail medical management⁹ and become candidates for functional endoscopic sinus surgery (FESS). Many of these patients experiencing medical-treatment failures elect to not undergo surgery as it does not address the underlying inflammation of CRS nor obviate the need for continued medical therapy. Moreover, approximately 65% of patients have recurrent symptoms within the first year post-FESS.¹⁰

Therefore, novel therapeutic modalities are needed for CRS patients, with and without nasal polyps, that fail current medical management.

### SUMMARY OF THE INVENTION

The present invention relates to materials, devices, kits and methods for use in treatment of patients having a sinus condition, including those with (or at risk of having) severe chronic rhinosinusitis (CRS) symptoms. In one embodiment, the present invention contemplates a method of treating a sinus condition in a patient or patient population. In one embodiment, implants may be used to improve sinus patency, for example, in surgically modified sinus spaces or in spaces such as the middle meatus that have not previously undergone surgical modification. Moreover, these scaffolds may be used to deliver local therapeutic agent(s) to nasal spaces, such as mometasone furoate (MF) including, for instance, as part of a treatment program that is an alternative to sinus surgery (e.g., an alternative to FESS) or in other instances as part of a postoperative care of FESS in some embodiments. In one embodiment, implantable sinonasal treatment achieves 24 weeks of benefit (or more) from a single bilateral administration of MF-eluting implants in the first and second middle meatus of surgically naïve CRS patients with and without nasal polyps. In one embodiment, subjects continue to improve even after the implants are removed, showing a durable response. Importantly, removal was not associated with any immediate increase in adverse symptoms.

In one embodiment, said patient is a candidate for sinus surgery such as Functional Endoscopic Sinus surgery (FESS) based on a first testing score. In one embodiment, said first testing score is a 22-item Sinonasal Outcomes Test (SNOT-22) severity score greater than or equal to 20 prior to implantation of said first implant. In one embodiment, after bilateral implantation of MF-eluting implants said patient is no longer a candidate for sinus surgery such as Functional Endoscopic Sinus surgery (FESS) based on second testing score. In one embodiment, said second testing score is a 22-item Sinonasal Outcomes Test (SNOT-22) severity score less than 20 after implantation of said first implant. In one embodiment, 1 month after implantation of said first implant said patient is no longer a candidate for Functional Endoscopic Sinus surgery (FESS) based on said second testing score. In one embodiment, 3 months after implantation of said first implant said patient is no longer a candidate for Functional Endoscopic Sinus surgery (FESS) based on said second testing score. In one embodiment, 6 months after implantation of said first implant said patient is no longer a candidate for Functional Endoscopic Sinus surgery (FESS) based on said second testing score. Avoidance of surgical interventions in the treatment of CRS is preferred for patients since these procedures carry surgery-associated risks, cause post-operative pain and discomfort, and require burdensome and costly post-operative cleaning.

In some embodiments, scaffolds or implants can be placed bilaterally in the first and second middle meatus of a subject easily in the office setting (and later they can be easily removed). In some embodiments, a patient with a drug-eluting scaffold or implant experiences significant symptom relief by 7 days with a durable effect to at least 12 weeks, up to 16 weeks, up to 3 months, up to 4 months, up to 5 months, up to 6 months and beyond, in duration. In some embodiments, a patient with a scaffold or implant was a CRS surgical candidate patient prior to implantation with an implant or scaffold comprising mometasone furoate (MF) converted to a patient no longer requiring surgery (i.e. the patient's symptoms are reduced such that the patient no longer meets the criteria designating the patient as a surgical candidate). In some embodiments, a scaffold results in rapid symptom improvement as early as 1 week. In some embodiments, a profound effect is observed by 3 months with the majority of patients converted from being surgical candidates to no longer meeting the criteria for being a surgical candidate.

In one embodiment, the method comprises a) providing first and second implants, each comprising about 7500 micrograms of mometasone furoate; b) implanting said first implant inside a first middle meatus of a first patient (or first patient population) having a sinus condition; c) implanting said second implant inside a second middle meatus of said first patient (or first patient population); d) monitoring the first patient's (or first patient population's) sinus condition for a period of at least 12 weeks, wherein the first patient (or first patient population) implanted with first and second implants each comprising about 7500 micrograms of mometasone furoate exhibits a reduction in the need for rescue treatment when compared to a second patient (or second patient population) having a sinus condition implanted with first and second implants each comprising about 2500 micrograms of mometasone furoate. Monitoring can be intermittently performed, e.g. once a week, once every two weeks, once every four weeks; it need not be continuous. Monitoring can be done by a physician, nurse, technician or other medical staff. Monitoring can be done in person, e.g. in a medical office, or remotely, e.g. by phone. Monitoring can be as simple as obtaining the subjective report from the patient on their status, e.g. level and number of symptoms, if any.

It is not intended that the present invention be limited to the patient (or patient population) in terms of prior treatment. Nonetheless, in a preferred embodiment, the first patients have failed medical management (e.g. with topical steroids) and become candidates for functional endoscopic sinus surgery (FESS). In a preferred embodiment, the patient (or patient population) is surgically naive, i.e. they have not had any prior sinus/nasal surgery. In one embodiment, the patients have or are without nasal polyps.

It is not intended that the present invention be limited to the nature of the rescue treatment. In one embodiment, the rescue treatment is a medication. Such rescue medications may include intranasal corticosteroids (INCS), oral corticosteroids, antibiotics, antihistamines, oral decongestants, and monoclonal antibodies.

It is not intended that the present invention be limited to the nature of the drug release kinetics of the implant. Nonetheless, in a preferred embodiment, the drug is released in a substantially linear manner over at least 12 weeks of the implantation, e.g. from the second week to the 13^{th} week. In a more preferred embodiment, the implant exhibits a zero-order release over at least 12 weeks of the implantation, e.g. from the second week to the 13th week. In one embodiment, said first and second implants are configured to release 20 to 80% of said mometasone furoate during the first 12 weeks. In one embodiment, wherein 80% of said mometasone furoate is released over 24 weeks.

It is not intended that the response to the about 7500 micrograms of drug (in each implant) is manifested only in terms of reduced rescue treatment. In one embodiment, the sinus condition of said first patient (or first patient population) improves more quickly than a second patient (or second patient population) implanted with first and second implants comprising 2500 micrograms of mometasone furoate. In one embodiment, the first patient (or first patient population) experiences fewer headaches overall for LYR-210 (7500 μg) over both LYR-210 (2500 μg) and saline. In one embodiment, the first patient (or first patient population) has fewer Treatment-related Adverse events for LYR-210 (7500 μg) over LYR-210 (2500 μg). In one embodiment, the first patient (or first patient population) exhibit symptom improvement in nasal blockage, facial pain/pressure and/or nasal discharge for LYR-210 (7500 μg) over both LYR-210 (2500 μg) and nasal saline. In one embodiment, the first patient (or first patient population) exhibit symptom improvement in loss of smell for LYR-210 (7500 μg) over both LYR-210 (2500 μg) and controls with only saline treatment. In one embodiment, the first patient (or first patient population) exhibit symptom improvement measured by the SNOT-22 score with LYR-210 (7500 μg) over LYR-210 (2500 μg). In one embodiment, the first patient (or first patient population) shows greater early SNOT-22 improvement of LYR-210 (7500 μg) in patients having polyps over LYR-210 (2500 μg). In one embodiment, the first patient (or first patient population) shows a greater reduction in ethmoid sinus inflammation by LYR-210 (7500 μg) over both LYR-210 (2500 μg) and the control with only saline treatment.

In one embodiment, said sinus condition is a chronic sinus condition. In one embodiment, said sinus condition is characterized by at least two symptoms selected from the group consisting of nasal obstruction, nasal congestion, difficulty breathing through nasal passages, nasal polyp, nasal discharge, loss of smell, and facial pain. In one embodiment, CRS is characterized by 2 or more of the following symptoms (lasting at least 12 weeks): nasal discharge (rhinorrhea or post-nasal drip), nasal obstruction or congestion, hyposmia, and facial pressure or pain.

It is not intended that the present invention be limited to a particular structure for the implant. Nonetheless, in a preferred embodiment, at least one of said first or second implants is a braided structure. In a preferred embodiment, at least one of said first or second implants is a tubular structure. In a preferred embodiment, at least one of said first or second implants is self-expanding. In another embodiment, said implant comprises helical strands. An exemplary implant is shown in FIG. 9C. In another embodiment, the implant is a sheet. The permeable or semi-permeable sheets may be implanted flat or in a rolled state. In the rolled embodiment, the rolled sheet comprises an internal lumen. In this exemplary embodiment, the drug delivery device is comprised of a semi-permeable polymeric hollow sheet filled with a drug or active pharmaceutical ingredient (API) in the absence or presence of an osmogen. Examples are shown in FIGS. 11-12.

It is not intended that the monitoring be limited to just 12 weeks. In another embodiment, said monitoring of the first patient's sinus condition is done for a period of at least 16 weeks. In yet another embodiment, said monitoring of the first patient's sinus condition is done for a period of at least 20 weeks. In yet another embodiment, said monitoring is done for a period of 24 weeks or more. Monitoring can be intermittently performed, e.g. once a week, once every two weeks, once every four weeks; it need not be continuous. Monitoring can be done by a physician, nurse, technician or other medical staff. Monitoring can be done in person, e.g. in a medical office, or remotely, e.g. by phone. Monitoring can be as simple as obtaining the subjective report from the patient on their status, e.g. level and number of symptoms, if any.

It is not intended that the present invention be limited to how the implant is loaded with drug. However, in a preferred embodiment, said first and second implants each comprise at least one coating, said coating containing about 7500 micrograms of mometasone furoate, meaning between 7000 micrograms and 8000 micrograms, and more typically 7500 micrograms plus or minus 10%. In a preferred embodiment, said coating is a polymer coating. In a further embodiment, the drug containing coating is overlaid (at least in part) with another polymer coating or “topcoat” lacking drug. In one embodiment, the thickness of the topcoat controls the amount and/or timing of drug release.

In one embodiment, the present invention contemplates a method of treating a sinus condition, comprising a) providing first and second implants, each comprising about 7500 micrograms of mometasone furoate; b) implanting said first implant inside a first middle meatus of a first patient (or first patient population) having a sinus condition; c) implanting said second implant inside a second middle meatus of said first patient (or first patient population); d) monitoring the first patient's (or first patient population's) sinus condition for a period of at least 12 weeks, wherein the first patient (or first patient population) implanted with first and second implants each comprising about 7500 micrograms of mometasone furoate exhibits a reduction in the need for rescue treatment when compared to a second patient (or second patient population) having a sinus condition given only saline irrigation treatment and no mometasone furoate. Monitoring can be intermittently performed, e.g. once a week, once every two weeks, once every four weeks; it need not be continuous. Monitoring can be done by a physician, nurse, technician or other medical staff. Monitoring can be done in person, e.g. in a medical office, or remotely, e.g. by phone. Monitoring can be as simple as obtaining the subjective report from the patient on their status, e.g. level and number of symptoms, if any.

It is not intended that the present invention be limited to the patient (or patient population) in terms of prior treatment. Nonetheless, in a preferred embodiment, the first patients have failed medical management (e.g. with topical steroids) and become candidates for functional endoscopic sinus surgery (FESS). In a preferred embodiment, the patient (or patient population) is surgically naive, i.e. they have not had any prior sinus/nasal surgery. In one embodiment, the patients have or are without nasal polyps. In one embodiment, CRS is characterized by 2 or more of the following symptoms (lasting at least 12 weeks): nasal discharge (rhinorrhea or post-nasal drip), nasal obstruction or congestion, hyposmia, and facial pressure or pain

It is not intended that the present invention be limited to the nature of the rescue treatment. In one embodiment, the rescue treatment is a medication. Such rescue medications may include intranasal corticosteroids (INCS), oral corticosteroids, antibiotics, antihistamines, oral decongestants, and monoclonal antibodies.

It is not intended that the present invention be limited to the nature of the drug release kinetics of the implant. Nonetheless, in a preferred embodiment, the drug is released in a substantially linear manner over at least 12 weeks of the implantation, e.g. from the second week to the 13th week. In a more preferred embodiment, the implant exhibits a zero-order release over at least 12 weeks of the implantation, e.g. from the second week to the 13th week. In one embodiment, said first and second implants are configured to release 20 to 80% of said mometasone furoate during the first 12 weeks.

In one embodiment, the sinus condition of said first patient (or first patient population) improves when compared to said second patient. It is not intended that the present invention be limited to how this improvement is manifested. In one embodiment, the first patient (or first patient population) experiences fewer headaches overall for LYR-210 (7500 μg) over patients receiving only saline. In one embodiment, the first patient (or first patient population) exhibit symptom improvement in nasal blockage, facial pain/pressure and/or nasal discharge for LYR-210 (7500 μg) over patients receiving only saline. In one embodiment, the first patient (or first patient population) exhibit symptom improvement in loss of smell for LYR-210 (7500 μg) over controls with only saline treatment. In one embodiment, the first patient (or first patient population) shows a greater reduction in ethmoid sinus inflammation by LYR-210 (7500 μg) over the controls with only saline treatment.

In one embodiment, said sinus condition is a chronic sinus condition. In one embodiment, said sinus condition is characterized by at least two symptoms selected from the group consisting of nasal obstruction, nasal congestion, difficulty breathing through nasal passages, nasal polyp, nasal discharge, loss of smell and facial pain. In one embodiment, CRS is characterized by 2 or more of the following symptoms (lasting at least 12 weeks): nasal discharge (rhinorrhea or post-nasal drip), nasal obstruction or congestion, hyposmia, and facial pressure or pain.

It is not intended that the present invention be limited to a particular structure for the implant. Nonetheless, in a preferred embodiment, at least one of said first or second implants is a braided structure. In a preferred embodiment, at least one of said first or second implants is a tubular structure. In a preferred embodiment, at least one of said first or second implants is self-expanding. In another embodiment, said implant comprises helical strands. An exemplarimplant is shown in FIG. 9C. In another embodiment, the implant is a sheet. The permeable or semi-permeable sheets may be implanted flat or in a rolled state. In the rolled embodiment, the rolled sheet comprises an internal lumen. In this exemplary embodiment, the drug delivery device is comprised of a semi-permeable polymeric hollow sheet filled with a drug or active pharmaceutical ingredient (API) in the absence or presence of an osmogen.

It is not intended that the monitoring be limited to just 12 weeks. In another embodiment, said monitoring of the first patient's sinus condition is done for a period of at least 16 weeks. In yet another embodiment, said monitoring of the first patient's sinus condition is done for a period of at least 20 weeks. In yet another embodiment, said monitoring is done for a period of 24 weeks or more. Monitoring can be intermittently performed, e.g. once a week, once every two weeks, once every four weeks; it need not be continuous. Monitoring can be done by a physician, nurse, technician or other medical staff. Monitoring can be done in person, e.g. in a medical office, or remotely, e.g. by phone. Monitoring can be as simple as obtaining the subjective report from the patient on their status, e.g. level and number of symptoms, if any.

It is not intended that the present invention be limited to how the implant is loaded with drug. However, in a preferred embodiment, said first and second implants each comprise at least one coating, said coating containing about 7500 micrograms of mometasone furoate (MF), meaning between 7000 micrograms and 8000 micrograms, and more typically 7500 micrograms plus or minus 10%. In a preferred embodiment, said coating is a polymer coating. In a further embodiment, the drug containing coating is overlaid (at least in part) with another polymer coating or “topcoat” lacking drug. In one embodiment, the thickness of the topcoat controls the amount and/or timing of drug release. Sustained delivery of MF to the sinonasal mucosa is achieved along with sustained symptom reduction from a single administration of implants (rather than re-administering drug repeatedly). In a preferred embodiment, the implants are delivered for the treatment of CRS in surgically naïve adult patients, with or without nasal polyps, including those that have failed medical management.

In one embodiment, the present invention contemplates a method of treating a sinus condition, comprising a) providing first and second implants, each comprising about 7500 micrograms of mometasone furoate; b) implanting said first implant inside a first middle meatus of a first patient (or first patient population) having a sinus condition; c) implanting said second implant inside a second middle meatus of said first patient (or first patient population); d) monitoring the first patient's (or first patient population's) sinus condition for a period of at least 8 weeks, wherein the first patient (or first patient population) implanted with first and second implants each comprising about 7500 micrograms of mometasone furoate improves more quickly than a second patient (or second patient population) with a sinus condition implanted with first and second implants each comprising about 2500 micrograms of mometasone furoate. Monitoring can be intermittently performed, e.g. once a week, once every two weeks, once every four weeks; it need not be continuous. Monitoring can be done by a physician, nurse, technician or other medical staff. Monitoring can be done in person, e.g. in a medical office, or remotely, e.g. by phone. Monitoring can be as simple as obtaining the subjective report from the patient on their status, e.g. level and number of symptoms, if any.

It is not intended that the present invention be limited to when improvement is detected. However, in one embodiment, said improvement in the first patient (or first patient population) implanted with first and second implants each comprising about 7500 micrograms of mometasone furoate, as compared to the second patient (or second patient population) implanted with first and second implants each comprising about 2500 micrograms of mometasone furoate, is observed at least as early as 8 weeks (or as early as 10, 12, 14 or 16 weeks) after implantation of said implants. Further, in one embodiment, said improvement in the first patient (or first patient population) implanted with first and second implants each comprising about 7500 micrograms of mometasone furoate, as compared to the second patient (or second patient population) implanted with first and second implants each comprising about 2500 micrograms of mometasone furoate, is observed as early as 4 weeks after implantation of said implants, but more typically at 8 weeks or later. Fewer acute exacerbations of chronic sinusitis occurred in the LYR-210 (7500 μg) treatment group during a 24-week treatment period.

It is not intended that the present invention be limited to a particular structure for the implant. Nonetheless, in a preferred embodiment, at least one of said first or second implants is a braided structure. In a preferred embodiment, at least one of said first or second implants is a tubular structure. In a preferred embodiment, at least one of said first or second implants is self-expanding. In another embodiment, said implant comprises helical strands. In another embodiment, said implant comprises a sheet. In a further embodiment, said implant comprises a folded sheet. An exemplary implant is shown in FIG. 9C. In another embodiment, the implant is a sheet. The permeable or semi-permeable sheets may be implanted flat or in a rolled state. In the rolled embodiment, the rolled sheet comprises an internal lumen. In this exemplary embodiment, the drug delivery device is comprised of a semi-permeable polymeric hollow sheet filled with a drug or active pharmaceutical ingredient (API) in the absence or presence of an osmogen.

It is not intended that the monitoring be limited to just 8 weeks. In another embodiment, said monitoring of the first patient's sinus condition is done for a period of at least 12 weeks. In yet another embodiment, said monitoring of the first patient's sinus condition is done for a period of at least 16 weeks. In yet another embodiment, said monitoring is done for a period of 20 weeks or more. Monitoring can be intermittently performed, e.g. once a week, once every two weeks, once every four weeks; it need not be continuous. Monitoring can be done by a physician, nurse, technician or other medical staff. Monitoring can be done in person, e.g. in a medical office, or remotely, e.g. by phone.

It is not intended that the present invention be limited to how the implant is loaded with drug. However, in a preferred embodiment, said first and second implants each comprise at least one coating, said coating containing about 7500 micrograms of mometasone furoate, meaning between 7000 micrograms and 8000 micrograms, and more typically 7500 micrograms plus or minus 10%. In a preferred embodiment, said coating is a polymer coating. In a further embodiment, the drug containing coating is overlaid (at least in part) with another polymer coating or “topcoat” lacking drug. In one embodiment, the thickness of the topcoat controls the amount and/or timing of drug release. In one embodiment, said first and second implants are configured to release 20 to 80% of said mometasone furoate during the first 12 weeks.

In one embodiment, said sinus condition is a chronic sinus condition. In one embodiment, said sinus condition is characterized by at least two symptoms selected from the group consisting of nasal obstruction, nasal congestion, difficulty breathing through nasal passages, nasal polyp, nasal discharge, loss of smell and facial pain. In one embodiment, CRS is characterized by 2 or more of the following symptoms (lasting at least 12 weeks): nasal discharge (rhinorrhea or post-nasal drip), nasal obstruction or congestion, hyposmia, and facial pressure or pain.

In one embodiment, the present invention contemplates a self-expanding implantable device comprising about 7500 micrograms of mometasone furoate. It is not intended that the present invention be limited to the amount of self-expanding. Nonetheless, in one embodiment, the devices of the present disclosure preferably expand to from 70 to 100% of their as-manufactured configuration after being crimped (e.g. crimped to a smaller shape to facilitate delivery).

It is not intended that the present invention be limited to a particular structure for the device. Nonetheless, in a preferred embodiment, said device is a braided structure. In a preferred embodiment, said device is a tubular structure. In a preferred embodiment, said device comprises helical strands. In a preferred embodiment, said device is configured to conform to the middle meatus space. In certain embodiments, the device is configured to have a relatively high RRF to be able to hold open nasal features, such as the middle meatus, yet have a relatively low COF so as to avoid applying possibly injurious forces against the walls of the middle meatus. An exemplary device is shown in FIG. 9C. In one embodiment, the device is part of a kit that includes a delivery device.

It is not intended that the present invention be limited to how the device is loaded with drug. However, in a preferred embodiment, said device comprises at least one coating, said coating containing about 7500 micrograms of mometasone furoate, meaning between 7000 micrograms and 8000 micrograms, and more typically 7500 micrograms plus or minus 10%. In a preferred embodiment, said coating is a polymer coating. In a further embodiment, the drug containing coating is overlaid (at least in part) with another polymer coating or “topcoat” lacking drug. In one embodiment, the thickness of the topcoat controls the amount and/or timing of drug release.

It is not intended that the present invention be limited to the nature of the drug release kinetics of the implant. Nonetheless, in a preferred embodiment, the drug is released in a substantially linear manner over at least 12 weeks of the implantation, e.g. from the second week to the 13th week. In a more preferred embodiment, the implant exhibits a zero-order release over at least 12 weeks of the implantation, e.g. from the second week to the 13th week. In one embodiment, said device is configured to release 20 to 80% of said mometasone furoate during the first 12 weeks.

In one embodiment, the present invention contemplates a scaffold that conforms to the shape of the middle meatus space comprising: a) a scaffold comprising a plurality of polymeric strands that comprise a first polymer material; b) a coating over the scaffold that comprises a crosslinked elastomer; and c) a layer comprising about 7500 micrograms of mometasone furoate. In one embodiment, the device further comprises d) a topcoat over said layer comprising mometasone furoate, wherein the thickness of said topcoat is configured so that said mometasone furoate is to be released substantially linearly for more than 6 weeks after placement of the scaffold in the middle meatus. In one embodiment, said topcoat contains no drug. In one embodiment, the substantially linear release is after week one. In one embodiment, the polymer material comprises poly(lactide-co-glycolide). In one embodiment, the elastomer material comprises poly(lactide-co-caprolactone). In one embodiment, the elastomer material comprises poly(lactide-co-caprolactone) having molar percentage of lactide ranging from 30 to 50% and a molar percentage of caprolactone ranging from 50 to 70%. In one embodiment, the present invention contemplates delivering said scaffold to the middle meatus space of a human. In one embodiment, the human has a sinus condition. In one embodiment, the sinus condition is chronic. In one embodiment, the present invention contemplates bilateral delivery of first and second scaffolds, each comprising about 7500 micrograms of mometasone furoate, to the first and second middle meatus of a human. In one embodiment, the human has a sinus condition. In one embodiment, the sinus condition is chronic.

In one embodiment, the present invention contemplates a combination therapy for use in a method of treating a sinus condition, comprising first and second implants, each comprising at least one coating containing about 7500 micrograms of mometasone furoate, wherein the first implant is configured to fit inside a first middle meatus of a patient, the mometasone furoate configured to be released into a first nasal cavity for more than 12 weeks; and wherein the second implant is configured to fit inside a second middle meatus of said patient, the mometasone furoate configured to be released into a second nasal cavity for more than 12 weeks.

In one embodiment, the present invention contemplates an implant configured to fit inside the middle meatus, said implant comprising a coating comprising about 7500 micrograms of mometasone furoate, said implant configured to exhibit have a zero-order release for at least 60% of said mometasone furoate. In one embodiment, said implant is configured to exhibit a zero-order release between 1 and 12 weeks. In one embodiment, said implant is configured to exhibit a zero-order release between days 20 and 55, after implantation. In one embodiment, the present invention contemplates delivering said implant to the middle meatus space of a human. In one embodiment, the human has a sinus condition. In one embodiment, the sinus condition is chronic. In one embodiment, the present invention contemplates bilateral delivery of first and second implants, each comprising about 7500 micrograms of mometasone furoate, to the first and second middle meatus of a human. In one embodiment, the human has a sinus condition. In one embodiment, the sinus condition is chronic.

In one embodiment, the present invention contemplates an implant configured to fit inside the middle meatus, said implant comprising a coating comprising about 7500 micrograms of mometasone furoate, said implant configured to result in an approximately constant plasma concentration of mometasone furoate of less than 100 picograms/ml. In one embodiment, the approximately constant plasma concentration of mometasone furoate is 60 picograms/ml or less after week 1, and typically about 50 picograms/ml or less after week 1 (as shown in FIG. 10).

In one embodiment, the present invention contemplates delivering said implant to the middle meatus space of a human. In one embodiment, the human has a sinus condition. In one embodiment, the sinus condition is chronic. In one embodiment, the present invention contemplates bilateral delivery of first and second scaffolds, each comprising about 7500 micrograms of mometasone furoate, to the first and second middle meatus of a human. In one embodiment, the human has a sinus condition. In one embodiment, the sinus condition is chronic.

The present invention contemplates in one embodiment that a patient will continue to improve for a period of time in terms of their sinus condition even after the implants are removed (e.g. 2-4 weeks, 4-8 weeks, and even 16 weeks after removal). Thus, in one embodiment, the present invention contemplates a method of treating a sinus condition, comprising: a) providing first and second implants, each comprising mometasone furoate; b) implanting said first implant inside a first middle meatus of a patient having a sinus condition; c) implanting said second implant inside a second middle meatus of said patient; d) removing said first and second implants from said patient; and e) detecting improvement in said patient's sinus condition after said implants are removed for a period of at least 4 weeks. In one embodiment, the patient is implanted with first and second implants each comprising about 7500 micrograms of mometasone furoate. In one embodiment, the patient is implanted with first and second implants each comprising about 2500 micrograms of mometasone furoate. In one embodiment, said improvement detected in said patient's sinus condition is reduced nasal blockage, facial pain, nasal discharge (anterior/posterior), and/or loss of smell. Improvement can be in terms of one or all of these symptoms. In one embodiment, said patient's condition continues to improve for a period of at least 8 weeks after the implants are removed. In one embodiment, said patient's condition continues to improve for a period of at least 12 weeks after the implants are removed. In one embodiment, said patient's condition continues to improve for a period of at least 16 weeks after the implants are removed. It is not intended that the present invention be limited to any precise timepoint at which the implants are removed. However, in a preferred embodiment, said implants are removed after 12 weeks, 16 weeks, 20 weeks, 24 weeks or more.

The present invention contemplates in one embodiment that a patient's symptoms will be maintained at the level of the final week of treatment (e.g. based on a SNOT score), after the implants are removed, for a period of time (e.g. 2-4 weeks, 4-8 weeks, and even 16 weeks after removal). This durable response shows that the approach has benefits even after the implants are removed. Thus, in one embodiment, the present invention contemplates a method of treating a sinus condition, comprising: a) providing first and second implants, each comprising mometasone furoate; b) implanting said first implant inside a first middle meatus of a patient having a sinus condition; c) implanting said second implant inside a second middle meatus of said patient; d) removing said first and second implants from said patient; and e) measuring symptoms in said patient after said implants are removed, wherein said symptoms maintain the same level (e.g. based on a SNOT score) for a period of at least 4 weeks after removal. In one embodiment, the patient is implanted with first and second implants each comprising about 7500 micrograms of mometasone furoate. In one embodiment, the patient is implanted with first and second implants each comprising about 2500 micrograms of mometasone furoate. In one embodiment, said symptoms measured comprise nasal blockage, facial pain, nasal discharge (anterior/posterior), and/or loss of smell. The maintained level can be in terms of one or all of these symptoms. In one embodiment, said patient's symptom levels are maintained for a period of at least 8 weeks after the implants are removed. In one embodiment, said patient's symptom levels are maintained for a period of at least 12 weeks after the implants are removed. In one embodiment, said patient's symptom levels are maintained for a period of at least 16 weeks after the implants are removed.

In one embodiment, the present invention contemplates an implant configured to fit inside the middle meatus for preventing the need for surgery in a subject who is a candidate for sinus surgery, said implant comprising a coating comprising about 7500 micrograms of mometasone furoate, said implant configured to exhibit a zero-order release for at least 60% of said mometasone furoate, characterized in that the implant is delivered to the subject prior to surgery. In one embodiment, the implant is configured for treating the for at least 4 months. In one embodiment, said sinus surgery is Functional Endoscopic Sinus surgery (FESS). In one embodiment, said implant is configured to release 20 to 80% of said mometasone furoate during the first 12 weeks. In one embodiment, said implant is configured to exhibit a zero-order release between 1 and 12 weeks. In one embodiment, said zero-order release is exhibited in vitro in pH 7.4 PBS buffer containing 2% SDS at 37° C. In one embodiment, said implant is for use in a method for improving sinus patency. In one embodiment, said implant is for use in a method of treating a chronic sinus condition. In one embodiment, said chronic sinus condition is characterized by at least two symptoms selected from the group consisting of nasal obstruction, nasal congestion, difficulty breathing through nasal passages, nasal polyp, nasal discharge, and facial pain. In one embodiment, said implant is a braided structure. In one embodiment, said implant is a tubular structure. In one embodiment, said implant comprises helical strands. In one embodiment, said implant is self-expanding. In one embodiment, said implant has a diameter of at least 13 mm. In one embodiment, said implant has a length of at least 10 mm. In one embodiment, the subject has a 22-item Sinonasal Outcomes Test (SNOT-22) severity score greater than or equal to 20.

In yet another embodiment, the present invention contemplates an implant configured to fit inside the middle meatus for preventing the need for surgery in a subject who is a candidate for Functional Endoscopic Sinus surgery (FESS), said implant comprising a coating comprising about 7500 micrograms of mometasone furoate, said implant configured to exhibit a zero-order release for at least 60% of said mometasone furoate, characterized in that the implant is delivered to the subject prior to surgery, wherein the subject is treated for at least 4 months. In one embodiment, said implant is configured to release 20 to 80% of said mometasone furoate during the first 12 weeks. In one embodiment, said implant is configured to exhibit a zero-order release between 1 and 12 weeks. In one embodiment, said zero-order release is exhibited in vitro in pH 7.4 PBS buffer containing 2% SDS at 37° C. In one embodiment, said implant is for use in a method for improving sinus patency. In one embodiment, said implant is for use in a method of treating a chronic sinus condition. In one embodiment, said chronic sinus condition is characterized by at least two symptoms selected from the group consisting of nasal obstruction, nasal congestion, difficulty breathing through nasal passages, nasal polyp, nasal discharge, and facial pain. In one embodiment, said implant is a braided structure. In one embodiment, said implant is a tubular structure. In one embodiment, said implant comprises helical strands. In one embodiment, said implant is self-expanding. In one embodiment, said implant has a diameter of at least 13mm. In one embodiment, said implant has a length of at least 10mm. In one embodiment, the subject has a 22-item Sinonasal Outcomes Test (SNOT-22) severity score greater than or equal to 20.

In one embodiment, the present invention contemplates a method of treating a sinus condition, comprising a) providing first and second implants, each comprising at least one coating containing about 7500 micrograms of mometasone furoate, and wherein the first implant is configured to fit inside a first middle meatus of a patient, and wherein the second implant is configured to fit inside a second middle meatus of said patient; b) implanting said first and second implants in a first and second middle meatus of a patient with symptoms of a sinus condition, and c) detecting a reduction in one or more symptoms, thereby treating said sinus condition. In one embodiment, the mometasone furoate configured in each implant exhibits a zero-order release for 12 weeks or more of a planned implantation period. In one embodiment, said reduction in one or more symptoms is reflected in a SNOT score. In one embodiment, said sinus condition is a chronic sinus condition. In one embodiment, said sinus condition is characterized by at least two symptoms selected from the group consisting of nasal obstruction, nasal congestion, difficulty breathing through nasal passages, nasal polyp, nasal discharge, loss of smell, and facial pain. In one embodiment, the implants comprise an additional coating that at least partially covers the drug-containing coating. In one embodiment, this additional coating lacks drug. In one embodiment at least one of said first or second implants is a braided structure. In one embodiment, at least one of said first or second implants is a tubular structure. In one embodiment, at least one of said first or second implants is self-expanding. In one embodiment, at least one of said first or second implants is configured as a sheet. In one embodiment, at least one of said first or second implants is configured as a rolled sheet. In one embodiment, said reduction comprises an improvement in a SNOT-22 scores by 50% (from baseline, i.e. the number measured prior to implantation or at the very beginning of the implantation period) at week 24 of the implantation period. In one embodiment, said reduction comprises no symptoms (or only mild symptoms) at week 24 of the implantation period.

In yet another embodiment, the present invention contemplates a method of reducing sleep dysfunction, comprising a) providing first and second implants, each comprising at least one coating containing about 7500 micrograms of mometasone furoate, and wherein the first implant is configured to fit inside a first middle meatus of a patient, and wherein the second implant is configured to fit inside a second middle meatus of said patient; b) implanting said first and second implants in a first and second middle meatus of a patient with symptoms of sleep dysfunction, and c) detecting a reduction in one or more symptoms, thereby treating said sleep dysfunction. In one embodiment, the symptoms of sleep dysfunction include difficulty falling asleep, waking up at night, lack of a good night's sleep, waking up tired, and fatigue. In one embodiment, said reduction in one or more symptoms is reflected in a SNOT score. In one embodiment, said subject with sleep dysfunction has a sinus condition. In one embodiment, said sinus condition is a chronic sinus condition. In one embodiment, said sinus condition is characterized by at least two symptoms selected from the group consisting of nasal obstruction, nasal congestion, difficulty breathing through nasal passages, nasal polyp, nasal discharge, loss of smell, and facial pain. In one embodiment, the implants comprise an additional coating that at least partially covers the drug-containing coating. In one embodiment, this additional coating lacks drug. In one embodiment at least one of said first or second implants is a braided structure. In one embodiment, at least one of said first or second implants is a tubular structure. In one embodiment, at least one of said first or second implants is self-expanding. In one embodiment, at least one of said first or second implants is configured as a sheet. In one embodiment, at least one of said first or second implants is configured as a rolled sheet. In one embodiment, said reduction comprises an improvement in a SNOT-22 scores by 50% (from baseline, i.e. the number measured prior to implantation or at the very beginning of the implantation period) at week 24 of the implantation period.

In one embodiment, the present invention contemplates a method of treating a sinus condition, a) providing first and second implants, each comprising at least one coating containing about 7500 micrograms of mometasone furoate, and wherein the first implant is configured to fit inside a first middle meatus of a patient, the mometasone furoate configured to have a zero-order release for 12 weeks or more of a planned implantation period; and wherein the second implant is configured to fit inside a second middle meatus of said patient, the mometasone furoate configured to have a zero-order release for 12 weeks or more of a planned implantation period, b) implanting said first and second implants in a first and second middle meatus of a patient with symptoms of a sinus condition, and c) detecting and/or measuring a reduction in one or more symptoms, thereby treating said sinus condition.

In another embodiment, the present invention contemplates a method of treating a sinus condition, a) implanting first and second implants in a first and second middle meatus of a patient with a sinus condition wherein said patient is a candidate for sinus surgery, each implant comprising at least one coating containing about 7500 micrograms of mometasone furoate, and b) detecting and/or measuring a reduction in one or more symptoms, wherein after 20-24 weeks (and more preferably after just 8 weeks, 10 weeks, 12 weeks or 16 weeks) said patient is no longer a candidate for sinus surgery, thereby treating said sinus condition. In one embodiment, said patient is a candidate for sinus surgery based on a SNOT score of symptoms. In one embodiment, said patient (after step b) is no longer a candidate for sinus surgery based on a SNOT score. In one embodiment, the patient is no longer a candidate for sinus surgery based on the 4 cardinal symptom composite score (4CS), which includes nasal blockage, nasal discharge, facial pain/pressure, and loss of smell. In one embodiment, said patient is no longer a candidate for sinus surgery based on the 3 cardinal symptom composite score (3CS), which includes nasal blockage, facial pain/pressure, and nasal discharge. In one embodiment, said patient is no longer a candidate for sinus surgery where 3CS≤4.

In one embodiment, the present invention contemplates a combination for preventing the need for surgery in a subject who is a candidate for sinus surgery [such as Functional Endoscopic Sinus surgery (NESS)], comprising first and second implants, each comprising at least one coating
containing about 7500 micrograms of mometasone furoate, wherein the first implant is configured to fit inside a first middle meatus of the subject, the mometasone furoate configured to have a zero-order release for more than 12 weeks; and wherein the second implant is configured to fit inside a second middle meatus of said subject, the mometasone furoate configured to have a zero-order release for more than 12 weeks, characterized in that the combination is delivered to the subject prior to surgery, wherein the subject is treated for at least 4 months. In one embodiment, said first and second implants are configured to release 20 to 80% of said mometasone furoate during the first 12 weeks. In one embodiment, said each of said first and second implants is configured to exhibit a zero-order release between 1 and 12 weeks. In one embodiment, said zero-order release is exhibited in vitro in pH 7.4 PBS buffer containing 2% SDS at 37° C. In one embodiment, said combination is for use in a method of treating a chronic sinus condition. In one embodiment, said chronic sinus condition is characterized by at least two symptoms selected from the group consisting of nasal obstruction, nasal congestion, difficulty breathing through nasal passages, nasal polyp, nasal discharge, and facial pain.

In one embodiment, at least one of said first or second implants is a braided structure. In one embodiment, at least one of said first or second implants is a tubular structure. In one embodiment, at least one of said first or second implants comprises helical strands. In one embodiment, at least one of said first or second implants is self-expanding.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Exemplary embodiments are illustrated in referenced figures. It is intended that the embodiments and figures disclosed herein are to be considered illustrative rather than restrictive.

FIG. 1 LANTERN Phase 2 Study Design Schematic; including LYR-210 implants ranging from 2500 μg-7500 μg.

FIG. 2 LANTERN Phase 2 Study Disposition of Patients (CONSORT) Diagram. ITT=Intention-to-treat.

FIG. 3A-C Patient symptom improvement in nasal blockage, facial pain/pressure, and nasal discharge. Mean change from baseline (CFBL) in the 7-day average score in (FIG. 3A) nasal blockage, (FIG. 3B) facial pain/pressure, and (FIG. 3C) nasal discharge (anterior/posterior). Data represents LSM. P<0.05 is considered statistically significant to control.

FIG. 4A-C Patient symptom improvement in loss of smell in patients with moderate-to-severe baseline disease. Mean change from baseline (CFBL) in the 7-day average score in loss of smell for patients with moderate-to-severe baseline anosmia (>2 baseline score in loss of sense of smell). FIG. 4A CFBL in 4CS Score. FIG. 4B CFBL in 3CS Score. FIG. 4C CFBL in loss of smell. LYR-210 (7500 μg) (n=15 patients), LYR-210 (2500 μg) (n=20 patients), control (n=20 patients). Data represents LSM. P<0.05 is considered statistically significant to control.

FIG. 5A-B Patient symptom improvement measured by the composite score of (FIG. 5A top) 4 cardinal symptoms (4CS) and (FIG. 5B) 3 cardinal symptoms (3CS). Mean change from baseline (CFBL) in the 7-day average score in 4CS include nasal blockage, facial pain, nasal discharge (anterior/posterior), and loss of smell. 3CS include nasal blockage, facial pain, and nasal discharge (anterior/posterior). Data represents LSM. P<0.05 is considered statistically significant to control. FIG. 5A bottom. Mean trends show lasting treatment effect post-removal of LYR-210. Approximately 73% mean compliance with daily nasal saline irrigation throughout the Post-Treatment Period.

*Excluded from post-treatment analysis: Patients who received rescue treatment prior to completing 80% of the post-treatment follow-up; and patients with missing data in the post-treatment period. FIG. 6A-C Patient symptom improvement measured by SNOT-22. (FIG. 6A) Mean change from baseline (CFBL) in SNOT-22 total score. Data represents LSM. P<0.05 is considered statistically significant to control. FIG. 6B Percent patients administered bilateral LYR-210 (2500 μg), bilateral LYR-210 (7500 μg), or control that achieved minimal clinically important difference (MCID) in SNOT-22 total score at week 24. FIG. 6C Percent patients with nasal polyps vs. without nasal polyps administered bilateral LYR-210 (2500 μg), bilateral LYR-210 (7500 μg), or control that achieved MCID in SNOT-22 total score at week 24.

FIG. 7 Reduction in ethmoid sinus inflammation measured by bilateral Zinreich MRI scores. Mean change from baseline (CFBL) in the bilateral ethmoid Zinreich score (composite score of anterior ethmoid and posterior ethmoid Zinreich scores) at 24 weeks. Data represents mean. P<0.05 is considered statistically significant to control.

FIG. 8 Time-to-first rescue treatment use over 24 weeks. An event is when a first rescue treatment was used. Patients who did not achieve the event were censored at the end of treatment date or at the early termination date. LYR-210 (7500 μg) (n=1 patient), LYR-210 (2500 μg) (n=2 patients), saline irrigation control (n=7 patients) used rescue treatment over the 24-week treatment period.

FIG. 9A is an illustration showing anatomical labeling, as a representation of a Coronal MRI image.

FIG. 9B illustrates, for reference, a magnified endoscopic view looking into a left (in relation to the patient) nostril, showing a septum (S), a middle turbinate (MT) and a middle meatus (MM).

FIG. 9C illustrates a partial deployment of the scaffold from the end of the applicator sheath, outside of nose, merely for illustrative purposes. LYR-210 matrix self-expands from a constrained state when deployed from an applicator.

FIG. 10 is a graph showing the resulting plasma concentration of mometasone furoate over time following the bilateral implantation of either 1) first and second implants, each comprising about 7500 micrograms of mometasone furoate or 2) first and second implants, each comprising about 2500 micrograms of mometasone furoate, in the middle meatus of subject having a sinus condition, e.g. CRS patients. The plasma concentration achieved remains relatively constant between days 5 and 55, indicating that the release of drug from the implant is substantially linear and even zero-order (e.g. for days 5-55, and particularly days 25-55). Plasma MF concentration-time profiles for LYR-210 (2500 μg) and LYR-210 (7500 μg) through 56 days. Data are represented as mean and standard deviation. Cₛₛ=steady state concentration.

FIG. 11 shows a schematic illustration of a rolled osmotic drug delivery sheet embodiment comprising one or more delivery orifices (500), a semi-permeable polymer membrane (501), an API (502) and an osmogen (503).

FIG. 12 shows a schematic illustration of a self-expandable implant embodiment comprising osmotic drug delivery fibers (600) comprising orifices (601).

### DEFINITIONS

As used herein, terms “sinus” and “sinus cavity” refer to cavities which include the maxillary, frontal and ethmoid sinuses, the ethmoid infundibulum and the sphenoid sinuses. The middle meatus refers to a nasal cavity that is not a sinus cavity. The ostiomeatal complex is a channel that links the frontal sinus, anterior ethmoid air cells and the maxillary sinus to the middle meatus, allowing airflow and mucociliary drainage.

As used herein, a “condition” refers to a particular state of being. One example is a “medical condition” referring to how good or bad is a person's physical state. A “medical condition” also is a broad term that includes disorders, diseases, lesions, and symptoms thereof. A medical condition may also include a specific type of area of the body, such as when using the term “sinus condition” referring to any one or more of symptoms including “cardinal” or “defining symptoms”.

“Chronic sinus condition” or “CRS” refers to inflammation of the nose and paranasal sinuses characterized by the presence of two or more of the following symptoms for greater than 12 weeks duration: 1) nasal blockage/obstruction/congestion; 2) nasal discharge; 3) facial pain/pressure; 4) reduction or loss of smell (ref-1). Objective confirmation of the diagnosis is made by sinus CT scan or nasal endoscopy that will also determine the phenotype: CRSsNP or CRSwNP. CRS was divided into two subtypes based on the presence of nasal polyps: CRS with nasal polyps (CRSwNP) and CRS without nasal polyps (CRSsNP). In adults, nasal polyps may be seen in both nasal passages and any unilateral polyps should be concerning for an alternative etiology such as malignancy.

“Chronic Sinusitis” refers to a sinus condition having at least two symptoms, including but not limited to: impaired nasal obstruction, congestion, nasal discharge when blowing nose, spontaneous nasal discharge from one or both nostrils, nasal discharge into the throat area, facial pain, facial pressure, facial fullness, headache, olfactory loss, etc. In some cases, CRS may be severe CRS.

Chronic rhinosinusitis with nasal polyps (CRSwNP) refers to a clinical diagnosis with the presence of subjective and/or objective evidence of chronic sinonasal inflammation.

A “nasal polyp” refers to an inflammatory lesion that projects into the nasal airway (cavity). Polyps are typically bilateral, however a polyp may also be found in one nasal airway, rather than both. Polyps may be soft, painless, noncancerous growths on the lining of nasal passages or sinuses. CRSwNP is frequently associated with asthma and allergic rhinitis.

“Cardinal symptom” refers to a symptom that may be diagnostic, and/or pathognomonic (of a sign or symptom) specifically characteristic or indicative of a particular disease or condition. Merely for example, a CRS may be one or more of nasal blockage, facial pain, nasal discharge (anterior/posterior), and loss of smell, including symptoms such as anterior or posterior rhinorrhea, nasal congestion, hyposmia and/or facial pressure or pain that last for greater than 12 weeks duration. The 4 cardinal symptom composite score includes nasal blockage, nasal discharge, facial pain/pressure, and loss of smell. The 3CS composite score includes nasal blockage, facial pain/pressure, and nasal discharge. In a preferred embodiment, 3CS=7-day average composite score of 3 cardinal symptoms of nasal blockage/obstruction/congestion, anterior/posterior nasal discharge, and facial pain/pressure

As used herein, “generally tubular” includes hollow shapes of circular cross-section or non-circular cross-section (e.g., oval, etc.) and hollow shapes of constant diameter or variable diameter (e.g. of tapered diameter, such as in a hollow frustum).

Implantable medical devices of certain embodiments of the present disclosure are generally self-expanding devices. In some embodiments, they are generally tubular devices, although some embodiments are contemplated to be configured as a sheet or folded sheet. In preferred embodiments, the devices comprise “strands” and “filaments. As used herein, “device,” “scaffold,” “stent”, “carrier” and “implant” may be used synonymously.

Scaffolds in accordance with certain embodiments of the present disclosure are provided with expansion and mechanical properties suitable to render the scaffolds effective for its intended purpose. Two measures of such mechanical properties that are used herein are “radial resistive force” (“RRF”) and “chronic outward force” (“COF”). RRF is the force that the scaffold applies in reaction to a crimping force, and COF is the force that the scaffold applies against a static abutting surface. In certain embodiments, the scaffolds are configured to have a relatively high RRF to be able to hold open bodily lumens, cavities, and nasal features, and the like, yet have a relatively low COF so as to avoid applying possibly injurious forces against the walls of bodily lumens, optic nerve, brain, or the like. For example, the scaffolds of the present disclosure preferably expand to from 70 to 100% of their as-manufactured configuration after being crimped, have an RRF ranging from 50 to 300 mmHg, and/or have an acute COF (at the time of delivery) ranging from 10 to 100 mmHg. Scaffolds in accordance with certain embodiments of the present disclosure are typically tubular devices which may be of various sizes, including a variety of diameters and lengths, and which may be used for a variety of sinus applications. In the case of objects of non-circular cross-section, “diameter” denotes width.

As used herein, “strength” and “stiffness” may be used synonymously to mean the resistance of the medical scaffolds of the present disclosure to deformation by radial forces or a force applied by the scaffolds against a static abutting object. Examples of strength and stiffness measurements, as used to characterize the medical scaffolds of the present disclosure, include radial resistive force and chronic outward force, as further described herein.

Implantable medical devices of certain embodiments of the present disclosure are generally self-expanding devices. As used herein, “self-expanding” is intended to include devices that are crimped to a reduced delivery configuration for delivery into the body, and thereafter tend to expand to a larger suitable configuration once released from the delivery configuration, either without the aid of any additional expansion devices or with the partial aid of balloon-assisted or similarly-assisted expansion. The many scaffold embodiments of the present disclosure are self-expanding in that they are manufactured at a first diameter, subsequently reduced or “crimped” to a second, reduced diameter for placement within a delivery catheter, and self-expand towards the first diameter when extruded from the delivery catheter at an implantation site. The first diameter may be at least 10% larger than the diameter of the bodily lumen into which it is implanted in some embodiments. The scaffold may be designed to recover at least about 70%, at least about 80%, at least about 90%, up to about 100% of its manufactured, first diameter, in some embodiments.

As used herein “strands” and “filaments” may be used interchangeably and include single fiber strands and filaments (also referred to as monofilaments) and multi-fiber strands and filaments. Helical strand. In some embodiments, which may be used in conjunction with any of the embodiments, a braided structure may comprise opposing sets of helical strands. For example, each set of helical strands may comprise between 2 and 64 members, more typically between 8 and 32 members.

In some preferred embodiments, an implant is a “long term” implant, such that an implant may be in contact with sinus tissue for up to 20 weeks, and more preferably 24 weeks or more. In some preferred embodiments, an implant is a “Long-acting” implant, such that a patient with an implant in contact with sinus tissue may show an effect of sinus tissue for up to 4 weeks, up to 8 weeks, up to 12 weeks, up to 16 weeks, up to 20 weeks, up to 24 weeks or more.

As used herein, “coding” refers to using a standardized (or chosen) terminology for converting a symptom, as an “adverse event” or “AE” into terminology for consistently analyze clinical data. An AE may also be described as a “Serious Adverse Event” or “SAE”. More specifically, MedDRA Code refers to an eight-digit number assigned to each term and is not to be confused with the text string of the term itself. Each term in MedDRA has a unique non-expressive 8-digit code used for data analysis.

As one example, a code was chosen for use as described in the Medical Dictionary for Regulatory Activities (MedDRA) Version 23.0. The MedDRA provides a general method of coding applicable to any phases of biopharmaceutical and medical product development, as briefly described herein. https://www.meddra.org/how-to-use/case-studies/industry-case-study https://www.meddra.org/glossary. One benefit of MedDRA is in its support of straightforward as well as sophisticated analyses. MedDRA can be used to analyze individual medical events (e.g., “Chronic sinusitis”) or issues involving a system, organ or aetiology (e.g., infections) using its hierarchical structure. AEs were coded using MedDRA Version 23.0.

“Monitoring” can be intermittently performed, e.g. once a week, once every two weeks, once every four weeks; it need not be continuous. Monitoring can be done by a physician, nurse, technician or other medical staff. Monitoring can be done in person, e.g. in a medical office, or remotely, e.g. by phone. Monitoring can be as simple as obtaining the subjective report from the patient on their status, e.g. level and number of symptoms, if any.

Terms such as “about” and “approximately” are intended, when used with a number, to indicate a range of plus/minus 5%.

### DESCRIPTION OF THE INVENTION

The present invention relates to materials, devices, kits and methods for use in treatment of patients having a sinus condition, including those with (or at risk of having) severe chronic rhinosinusitis (CRS) symptoms. In one embodiment, the present invention contemplates implants designed for use in patients with sinus conditions who have failed after standard medical treatment. In one embodiment, the present invention contemplates an implantable matrix (LYR-210) (7500 μg of MF) that locally elutes mometasone furoate (e.g. from the middle meatus to inflamed sinonasal tissue) for 8-12 weeks, and more preferably for 24 weeks or more in surgically naïve CRS patients with and without nasal polyps. In an open-label Phase 1 study, LYR-210 demonstrated clinically relevant improvement in the 22-item Sinonasal Outcome Test (SNOT-22). The safety and efficacy of LYR-210 (7500 μg) in CRS was further evaluated in the LANTERN Phase 2 study.

Methods: Sixty-seven surgically naïve adult CRS patients who failed previous medical management and were seeking further treatment were enrolled in a multicenter, blinded, controlled, dose-ranging LANTERN study. Patients had moderate-to-severe disease based on SNOT-22 and composite 7-day average scores of the 4 cardinal CRS symptoms (4C5), with diagnosis confirmed by nasal endoscopy and MRI. Patients were randomized (1:1:1) to saline-irrigation only control or bilateral in-office administration of LYR-210-2500 μg or LYR-210-7500 μg. Safety and efficacy were evaluated over 24 weeks.

LYR-210 (7500 μg) is an implantable drug matrix based on the XTreo™ drug delivery technology platform¹¹ that is designed to consistently elute mometasone furoate (MF), e.g. from the middle meatus (implant site) to local sinonasal mucosa for up to 24 weeks. LYR-210 is designed to fit within and dynamically conform to the middle meatus. The middle meatus allows for drainage of the paranasal sinuses and houses the osteomeatal complex, an originating site of mechanical obstruction leading to secondary infection in CRS.¹² The active ingredient, MF, is embedded in inactive ingredients composed of biocompatible and bioabsorbable polymers that aid in the controlled and sustained delivery of MF to the sinonasal mucosa from a single bilateral administration. LYR-210 (7500 μg) is in development for the treatment of CRS in surgically naïve adult patients that have failed medical management. In an open-label Phase 1 study (ClinicalTrials.gov Identifier: NCT02967731), LYR-210 was well-tolerated and demonstrated clinically relevant improvement in the 22-item Sino-nasal Outcome Test (SNOT-22) over 24 weeks in 20 surgically naive CRS patients.¹³ To further evaluate the safety and efficacy of LYR- 210, we conducted a multicenter, randomized, blinded, controlled, dose-ranging LANTERN Phase 2 study (ClinicalTrials.gov Identifier: NCT04041609).

Results: Both LYR-210 doses were safe and well-tolerated over the 24-week treatment period. LYR-210 demonstrated rapid and durable dose-dependent symptom improvement based on 4CS and SNOT-22, with LYR-210-7500 μg achieving statistical significance as early as 8 weeks and out to 24 weeks compared to control. LYR-210 (7500 μg) reduced rescue treatment use and radiographic ethmoid opacification at week 24 compared to control.

In one embodiment, the implants are removed and the patient continues to improve and/or maintains improvement in terms of reduced symptoms of a sinus condition. In one embodiment, the patient continued to improve for at least 2 weeks and even 4 weeks.

Importantly, removal was not associated with any immediate increase in adverse symptoms. Indeed, symptom levels were maintained, e.g. as measured by a SNOT and/or 4CS score.

### EXPERIMENTAL RESULTS

### Patient Characteristics

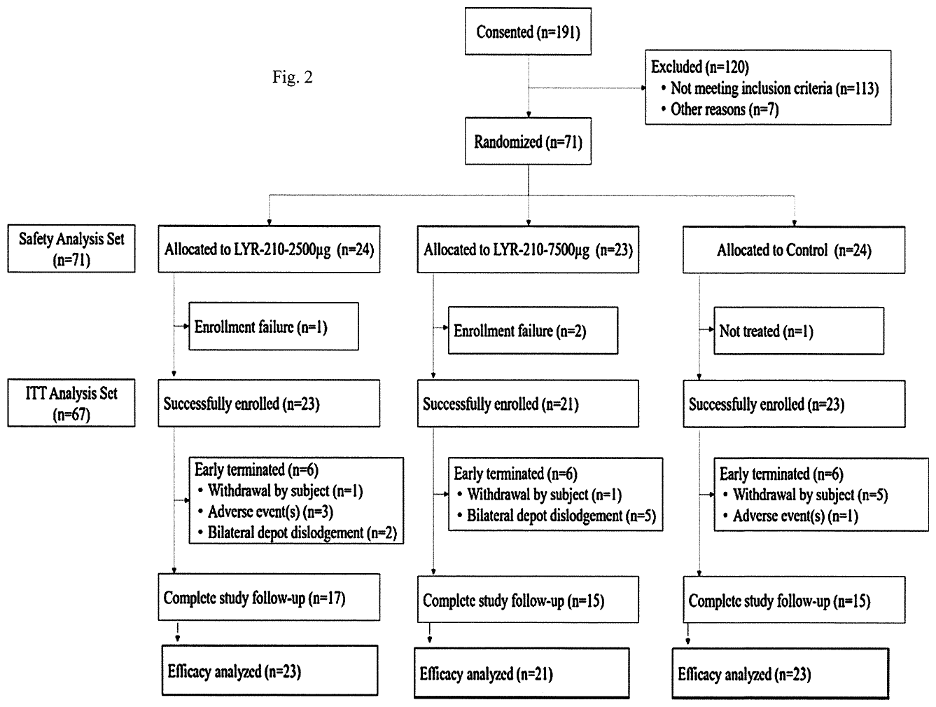

The LANTERN Phase 2 study planned to enroll up to 150 adult CRS patients that had failed medical management and had not undergone sinus surgery. Patient enrollment was curtailed early due to the COVID-19 global pandemic. Between May 2019 and Mar. 2020, 71 patients were randomized, 70 underwent an attempted study treatment procedure, and 67 were successfully enrolled. Of these, 23 patients were bilaterally administered LYR-210 (2500 μg), 21 were bilaterally administered LYR-210 (7500 μg), and 23 had a bilateral sham procedure (saline irrigation only control). Six patients in each study arm discontinued treatment before completing the planned 24 weeks. At week 22, 80% of administered LYR-210 drug matrices were retained. Disposition of patients is illustrated in FIG. 2.

Of the 67 successfully enrolled patients, 35 (52.2%) were male and 37 (55.2%) were diagnosed with bilateral nasal polyps by endoscopy, each representing approximately half the patients in each study arm. The nasal polyps were sizable, with the majority extending outside the middle meatus. Patients reported moderate-to-severe CRS symptoms¹⁶ at baseline with a mean SNOT-22 score of 68.2±18.4 (range 25 to 107) and a mean 4CS score of 9.7±1.59 (range 7.0 to 12.0). In addition, Patients exhibited sinonasal inflammation based on their baseline Zinreich scores (mean 20.3±12.33 (median 17, range 1-53)). Patients had at least 1 trial of INCS prior to screening, while 19.4%, 49.2%, and 73.1% of patients had previously used oral corticosteroids, antibiotics, and saline irrigations for CRS, respectively. Patient demographics, CRS medical history, and baseline clinical disease severity measures are summarized in Table 1.

TABLE 1
Patient demographics, medical history, and baseline characteristics.

| | LYR-210 | LYR-210 | | | |
|---|---|---|---|---|---|
| | (2500 μg) | (7500 μg) | Control | Total | |
| | (n = 23) | (n = 21) | (n = 23) | (n = 67) | P |
| Gender (male), n (%) | 11 (47.8) | 13 (61.9) | 11 (47.8) | 34 (52.2) | 0.565 |
| Age (years) | | | | | |
| Mean # SD | 42.3 ± 12.72 | 46.9 ± 12.72 | 41.3 ± 14.68 | 43.3 ± 13.44 | 0.357 |
| Median (range) | 41.0 (18- | 43.0 (28- | 43.0 (21- | 42.0 (18- | |
| | 72) | 75) | 78) | 78) | |
| Comorbidity, n (%) | | | | | |
| Seasonal allergic | 0 (0) | 2 (9.5) | 2 (8.7) | 4 (6.0) | 0.453 |
| rhinitis | | | | | |
| Perennial allergic | 2 (8.7) | 1 (4.8) | 4 (17.4) | 7 (10.4) | 0.431 |
| rhinitis | | | | | |
| Concurrent asthma | 3 (13.0) | 5 (23.8) | 4 (17.4) | 12 (17.9) | 0.670 |
| COPD | 1 (4.3) | 0 (0) | 0 (0) | 1 (1.5) | 1.000 |
| Sensitivity to NSAIDs | 2 (8.7) | 2 (9.5) | 1 (4.3) | 5 (7.5) | 0.734 |
| Current smoker | 2 (8.7) | 5 (23.8) | 3 (13.0) | 10 (14.9) | 0.353 |
| Nasal polyps, n (%) | 13 (56.5) | 11 (52.3) | 13 (56.5) | 37 (55.2) | 1.000 |
| Baseline SNOT-22 | | | | | |
| score | | | | | |
| Mean ± SD | 70.0 ± 19.92 | 62.9 ± 18.39 | 71.2 ± 16.57 | 68.2 ± 18.4 | 0.277 |
| Median (range) | 74.0 (41- | 65.0 (25- | 71.0 (47- | 69.0 (25- | |
| | 97) | 92) | 107) | 107) | |
| Baseline 4CS score | | | | | |
| Mean ± SD | 9.9 ± 1.61 | 9.3 ± 1.60 | 9.8 ± 1.58 | 9.7 ± 1.59 | 0.357 |
| Median (range) | 10.0 (7.0- | 8.9 (7.1-12.0) | 10.4 (7.3- | 9.7 (7.0- | |
| | 12.0) | | 12.0) | 12.0) | |

Baseline Modified Zinreich MRI score

| | | | | | |
|---|---|---|---|---|---|
| Mean ± SD | 19.6 ± 12.3 | 23.7 ± 12.4 | 17.6 ± 12.0 | 20.3 ± 12.3 | 0.268 |
| Median (range) | 16.0 (5- | 24.0 (5- | 15.5 (1- | 17.0 (1- | |
| | 52) | 53) | 49) | 53) | |

CRS medical treatment history, n (%)

| | | | | | |
|---|---|---|---|---|---|
| Intranasal | 23 (100) | 21 (100) | 23 (100) | 67 (100) | 1.000 |
| corticosteroid sprays | | | | | |
| Oral corticosteroids | 5 (21.7) | 5 (23.8) | 3 (13.0) | 13 (19.4) | 0.689 |
| Antibiotics | 13 (56.5) | 12 (57.1) | 8 (34.8) | 33 (49.2) | 0.267 |
| Saline irrigations | 15 (65.2) | 17 (81.0) | 17 (73.9) | 49 (73.1) | 0.543 |

COPD = chronic obstructive pulmonary disease; NSAIDs = nonsteroidal anti-inflammatory drugs;
4CS = composite score of 7-day average scores of 4 cardinal symptoms of chronic rhinosinusitis;
SD = standard deviation; SNOT-22 = 22-item Sino-Nasal Outcome Test.
Analysis of variance (ANOVA) is used to compare means of continuous variables between groups. Fisher's exact test is used to compare categorical variables between groups.

### Safety

Table 2 summarizes treatment emergent AEs reported by more than 1 patient in any study arm. The most common AEs reported in the LYR-210 (2500 μg) arm were chronic sinusitis, epistaxis and rhinorrhoea (n=4 each); in the LYR-210 (7500 μg) arm were chronic sinusitis and rhinitis (n=4 each); and in the control arm were chronic sinusitis (n=7). Other less common AEs included upper respiratory tract infection, oropharyngeal pain, nasal congestion, facial pain, dizziness, and hyperkalaemia. There was only one serious adverse event (SAE) reported in this study, in which a patient in the LYR-210 (2500vg) arm had acarodermatitis that was determined by the treating physician to be unrelated to study treatment.

The mean CFBL in TOP at week 24 was reduced in the LYR-210 (7500 μg) arm (−0.5±2.8, range −5.5 to 7.5) and in the LYR-210 (2500 μg) arm (−0.1±2.5, range −6.3 to 4.0) and was increased in the control arm (0.9±2.7, range −4.0 to 9.5). None of the patients in this study had a clinically significant increase in TOP. One patient in the control arm developed a clinically significant nuclear cataract while in this study. There was no significant decrease of morning serum cortisol levels at week 4, 12, or 24. No AEs indicative of adrenal insufficiency were reported.

TABLE 2
Treatment emergent adverse events and
serious adverse events summary.

| Event | LYR-210 | LYR-210 | |
|---|---|---|---|
| System Organ Class | (2500 μg) | (7500 μg) | Control |
| Preferred Term | n = 24 | n = 23 | n = 23 |
| AEs | | | |
| Infections and infestations | | | |
| Chronic sinusitis | 4 | 4 | 7 |
| Rhinitis | 1 | 4 | 0 |
| Upper respiratory tract infection | 2 | 1 | 1 |

Respiratory, thoracic and mediastinal disorders

| | | | |
|---|---|---|---|
| Epistaxis | 4 | 3 | 2 |
| Rhinorrhoea | 4 | 2 | 1 |
| Oropharyngeal pain | 0 | 1 | 2 |
| Nasal congestion | 2 | 0 | 0 |
| Nervous system disorders | | | |
| Headache | 2 | 1 | 3 |
| Dizziness | 2 | 0 | 0 |

General disorders and administration site conditions

| | | | |
|---|---|---|---|
| Facial pain | 1 | 2 | 0 |
| Metabolism and nutrition disorders | | | |
| Hyperkalaemia | 0 | 0 | 2 |
| SAEs | | | |

Infections and infestations

| | | | |
|---|---|---|---|
| Acarodermatitis | 1 | 0 | 0 |
| Treatment-related AEs | | | |
| Infections and infestations | | | |
| Rhinitis | 1 | 3 | 0 |

Respiratory, thoracic and mediastinal disorders

| | | | |
|---|---|---|---|
| Epistaxis | 3 | 1 | 0 |
| Rhinorrhoea | 2 | 0 | 0 |
| Nervous system disorders | | | |
| Headache | 0 | 0 | 2 |
| Procedure-related AEs | | | |
| Respiratory, thoracic and | | | |
| mediastinal disorders | | | |
| Epistaxis | 2 | 1 | 1 |

AE = Adverse Event; MedDRA = Medical dictionary for Regulatory Activities, SAE = Serious Adverse Event.
The Preferred Terms were summarized for AEs reported by more than 1 patient in any study group.
If a patient reported an AE more than once within that System Organ Class/Preferred Term, the patient was counted only once for that System Organ Class/Preferred Term.
The relatedness of an AE to study treatment or procedure was attributed by the treating physician.
AEs were coded using MedDRA Version 23.0.
* MedDRA Preferred Term for exacerbation/worsening of chronic sinusitis

### Efficacy

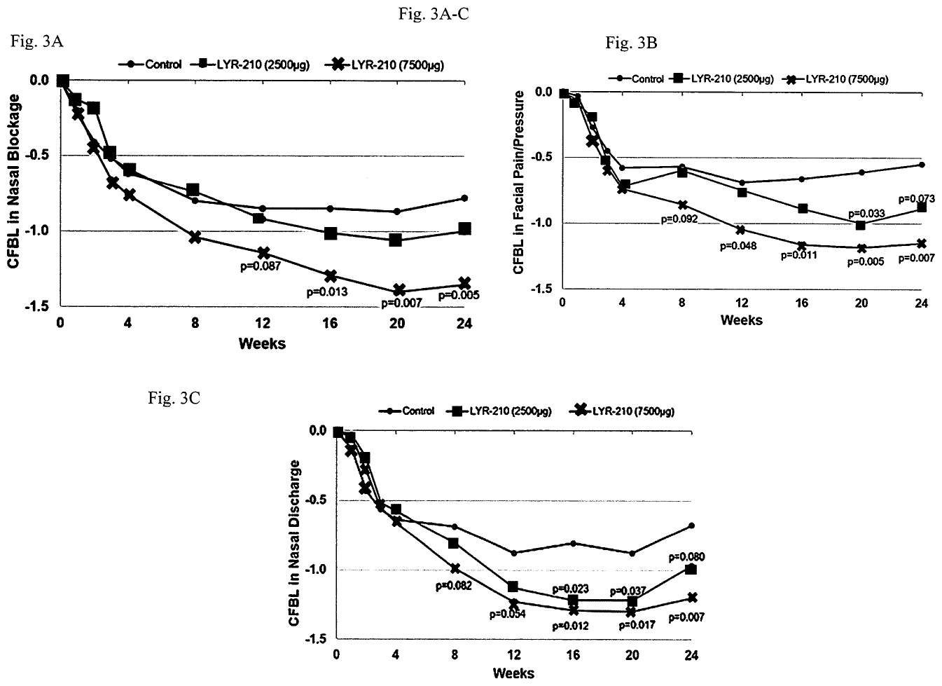

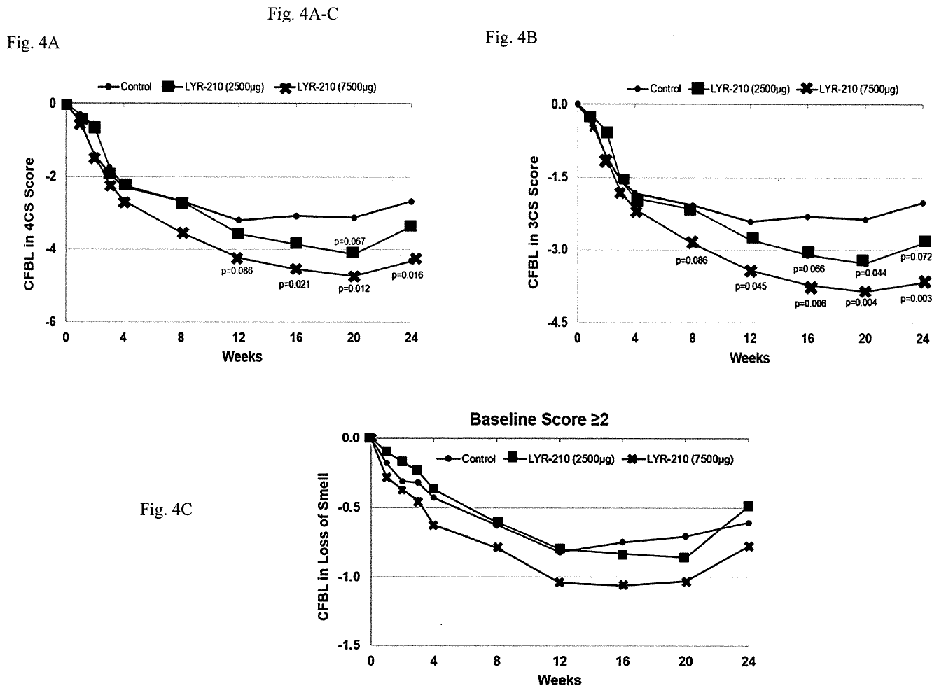

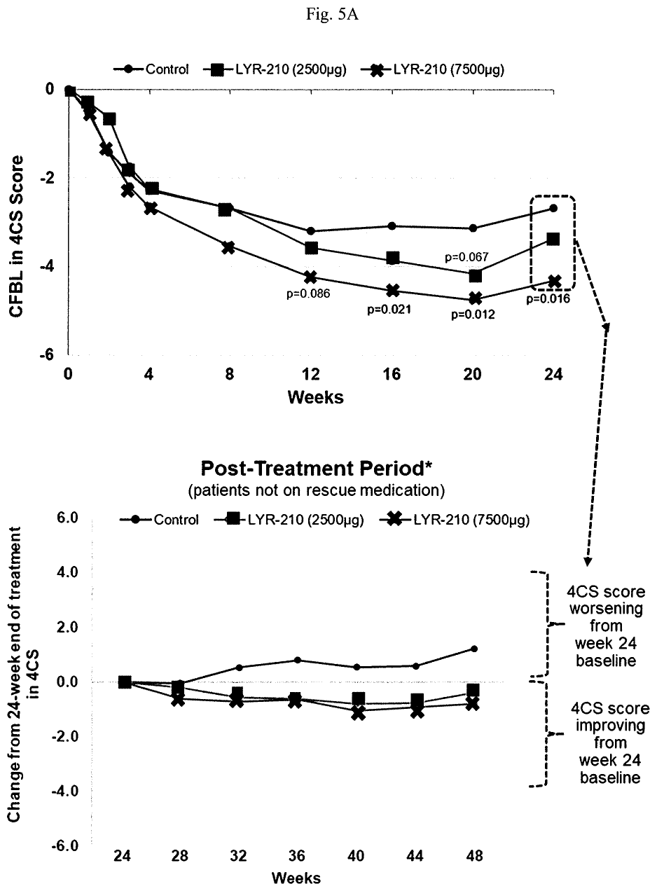

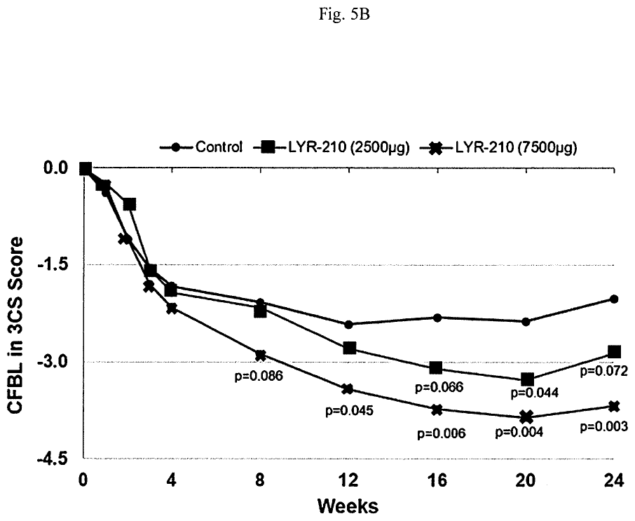

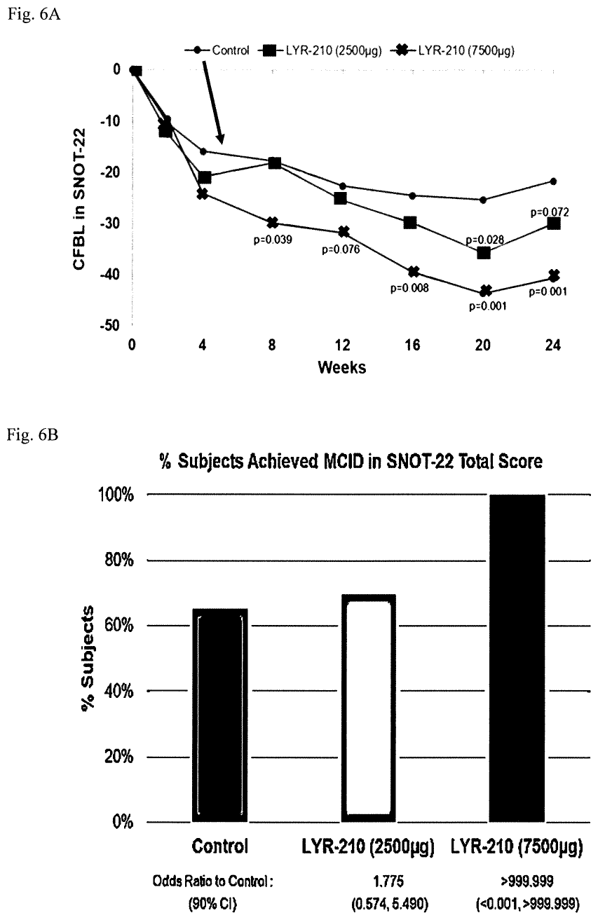

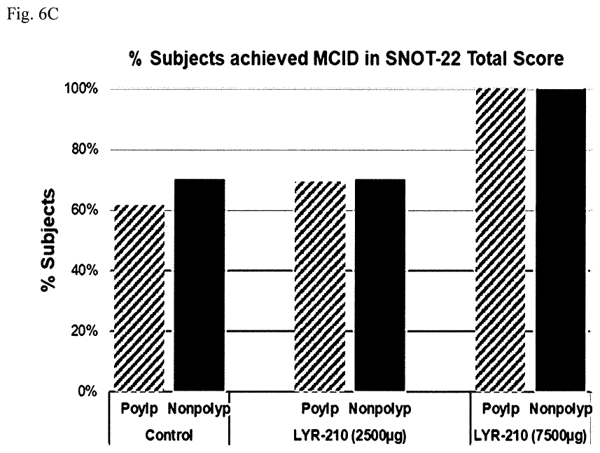

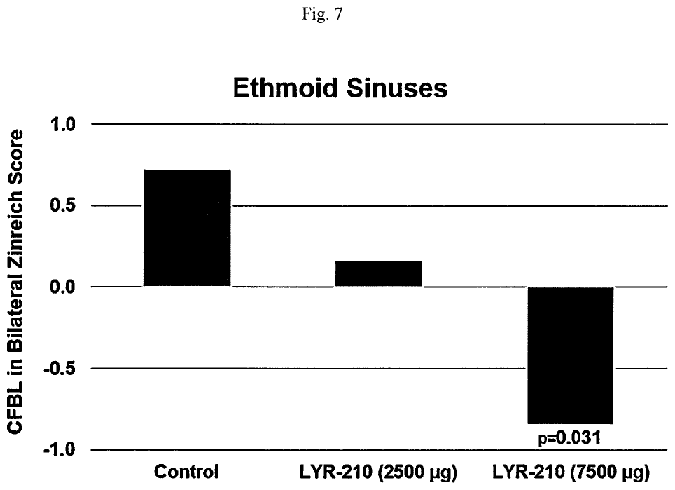

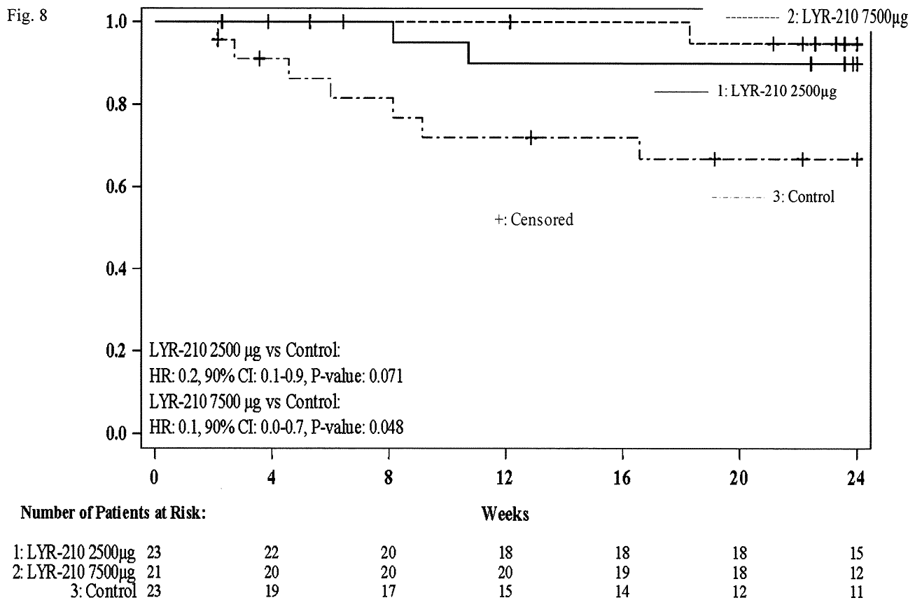

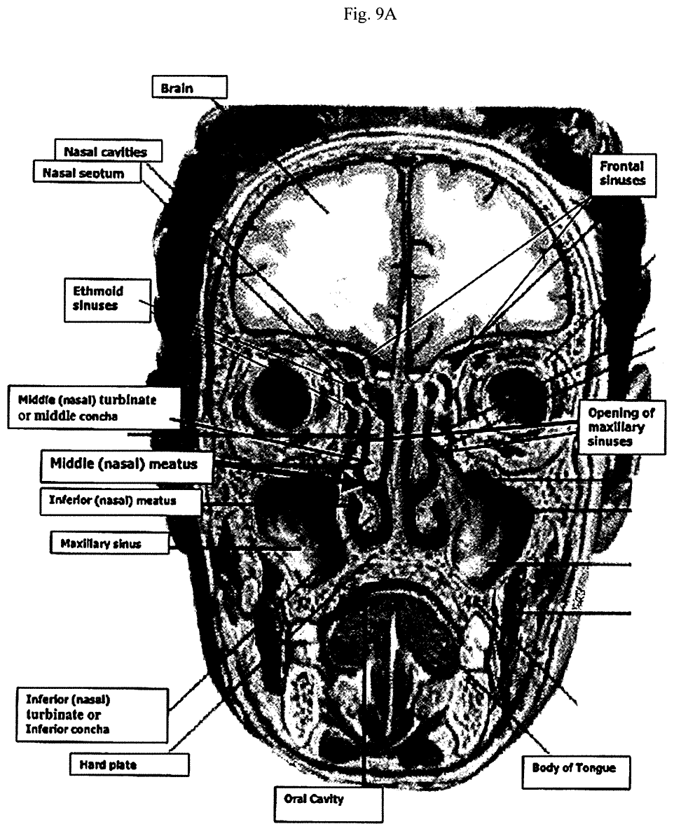

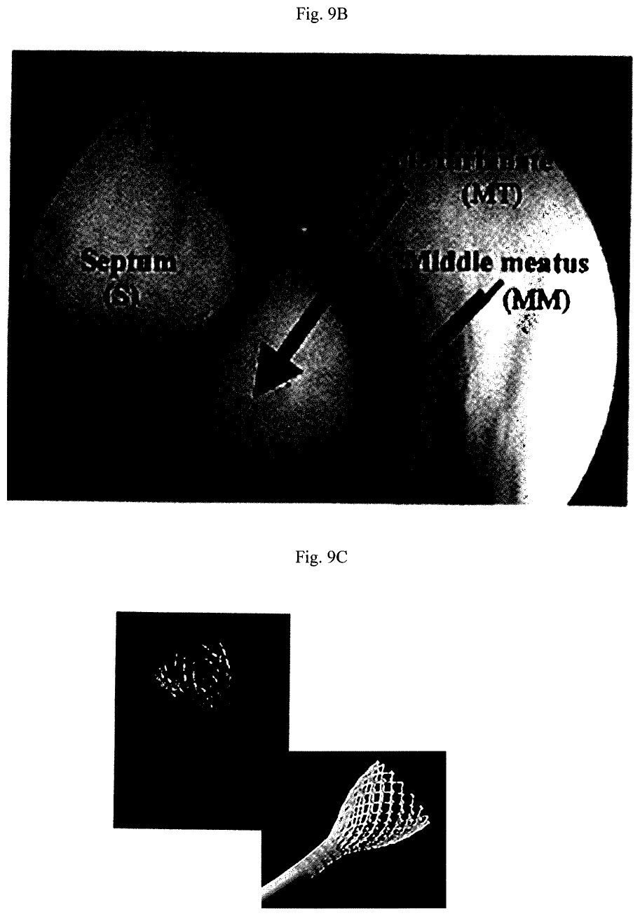

The treatment effect of LYR-210 was evaluated over 24 weeks based on patient symptom improvement, reduction in sinonasal inflammation, and reduction in the need for rescue treatment. LYR-210 demonstrated dose-dependent improvement in nasal blockage, facial pain/pressure, and nasal discharge in CRS patients throughout the 24-week treatment period (FIG. 3A-C). Specifically, LYR-210 (7500 μg) achieved statistically significant improvement compared to control in nasal blockage (weeks 16, 20, and 24) (FIG. 3A), facial pain/pressure (weeks 12, 16, 20, and 24) (FIG. 3B), and nasal discharge (weeks 16, 20, and 24) (FIG. 3C). Analysis of enrolled patients that exhibited moderate-to-severe anosmia at baseline, as defined by baseline score of ≥2 in loss of smell, showed that LYR-210 (7500 μg) improved patients' sense of smell (FIG. 4C).

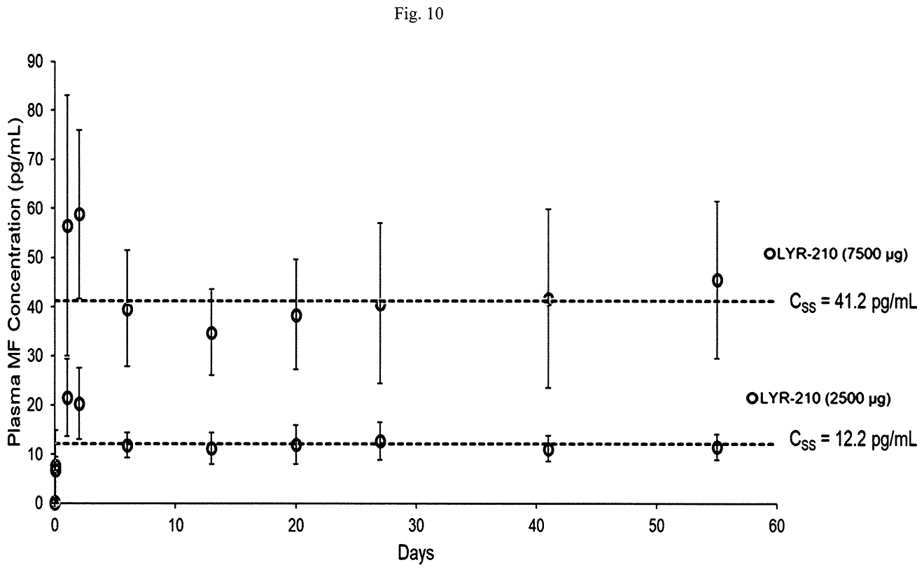

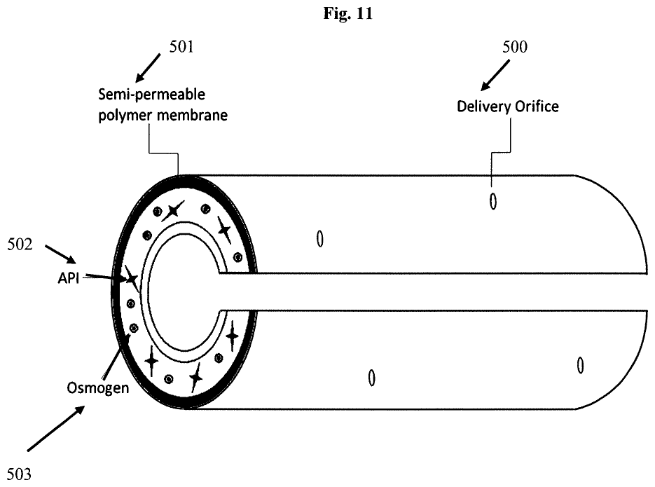

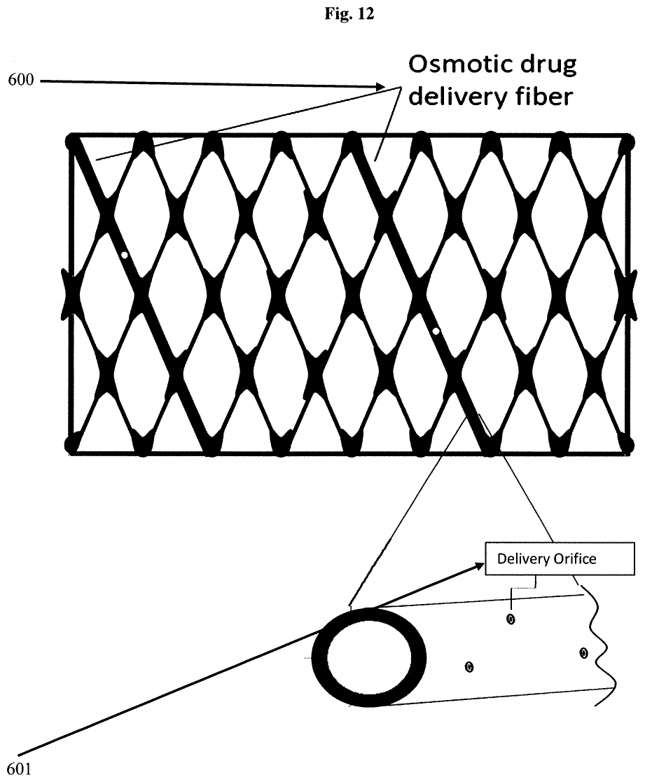

The 4 cardinal symptoms were analyzed throughout the 24-week treatment period as a composite score (4CS), which is the primary efficacy endpoint. The 4 cardinal symptom composite score, includes nasal blockage, nasal discharge, facial pain/pressure, and loss of smell. LYR-210 demonstrated dose-dependent improvement in the 4CS score that became more pronounced over 24 weeks (FIG. 5A upper graph). LYR-210 (7500 μg) demonstrated statistically significant improvement from baseline compared to control at weeks 16, 20, and 24. At week 24, patients had different stages of wellness, with LYR-210 (7500 μg) significantly improved over control.

To determine how LYR-210-treated patients responded after matrices were removed, their week 24 scores were set as a new baseline. (FIG. 5A lower graph) For this analysis, patients that required rescue treatment prior to completing 80% of the post-treatment follow-up, and those with missing data in the post-treatment period were excluded. Despite small numbers, the mean trends indicate lasting treatment effect post-removal of LYR-210, while the control showed worsening from week 24 baseline. Patients were provided nasal saline irrigation throughout the post-treatment period and were compliant with daily use approximately 73% of the time throughout these 24 weeks. There was an absence of a profound rebound effect in the post-treatment period. Thus, the implant can be removed and the patient can experience a lasting treatment effect and even continue to improve.

As only a subset of patients exhibited moderate-to-severe anosmia at baseline, we also analyzed the composite score of the 7-day average scores from nasal blockage, facial pain/pressure, and nasal discharge (3 cardinal symptoms (3CS)), which was not a prespecified endpoint in this study. LYR-210 demonstrated a dose-dependent treatment effect, with LYR-210 (7500 μg) achieving significant improvement in the 3CS score compared to control at week 12 through week 24 (FIG. 5B).

The SNOT-22 questionnaire is the clinical gold standard measurement of CRS patient burden and quality of life. 14 As shown in FIG. 6A, a dose response was observed between LYR-210 (7500 μg), LYR-210 (2500 μg) and control over the 24-week treatment period. LYR-210 demonstrated rapid, durable, and clinically meaningful improvement in SNOT-22 scores from baseline. Patients administered bilateral LYR-210 (7500 μg) exhibited statistically significant improvement compared to control at weeks 8, 16, 20, and 24. Notably, LYR-210 (7500 μg) achieved a 19-point improvement in SNOT-22 over control at week 24; more than 2-fold the minimal clinically important difference (MCID) of 8.9 points.¹⁴

SNOT-22 responder analysis revealed that LYR-210 (7500 μg) is superior at the percentage of subjects achieving MCID at week 24 [odds ratio >999.999 (<0.001, >999.999) compared to LYR-210 (2500 μg) and control] with 100% of patients administered LYR-210 (7500 μg) achieving≥MCID in SNOT-22 total score at week 24 compared to 70% in the LYR-210 (2500 μg) and 65% in the control arms (FIG. 6B-C).

Upon further assessment of patients administered LYR-210 (7500 μg), 70% achieved ≥MCID in SNOT-22 total score as early as week 4 (Table 3A). Importantly, this early onset was observed in patients with sizable nasal polyps and those without nasal polyps. 80% of CRSsNP patients and 60% of CRSwNP patients in the LYR-210 (7500 μg) arm achieved ≥MCD in SNOT-22 total score at week 4 (Table 3B). This strong treatment effect persisted throughout the study increasing to 100% of patients in the LYR-210 (7500 μg) arm achieving MCD in SNOT-22 total score by week 24 (Table 3A-Table 3B).

TABLE 3A
Percent (%) Subjects Achieved MCID in
SNOT-22 Total Score. LYR-210 7500 μg.

| Time Point | N | RSP | PCT |
|---|---|---|---|
| Week 2 | 19 | 9 | 47.4% |
| Week 4 | 20 | 14 | 70.0% |
| Week 8 | 20 | 16 | 80.0% |
| Week 12 | 20 | 17 | 85.0% |
| Week 16 | 20 | 18 | 90.0% |
| Week 20 | 20 | 19 | 95.0% |
| Week 24 | 20 | 20 | 100.0% |

TABLE 3B
Percent (%) Subjects Achieved MCID in SNOT-22
Total Score. LYR-210 7500 μg - polyp vs. nonpolyp.

| Time Point | Polyp Status | N | RSP | PCT |
|---|---|---|---|---|
| Week 2 | Polyp | 10 | 3 | 30.0% |
| | Nonpolyp | 9 | 6 | 66.7% |
| Week 4 | Polyp | 10 | 6 | 60.0% |
| | Nonpolyp | 10 | 8 | 80.0% |
| Week 8 | Polyp | 10 | 7 | 70.0% |
| | Nonpolyp | 10 | 9 | 90.0% |
| Week 12 | Polyp | 10 | 8 | 80.0% |
| | Nonpolyp | 10 | 9 | 90.0% |
| Week 16 | Polyp | 10 | 9 | 90.0% |
| | Nonpolyp | 10 | 9 | 90.0% |
| Week 20 | Polyp | 10 | 9 | 90.0% |
| | Nonpolyp | 10 | 10 | 100.0% |
| Week 24 | Polyp | 10 | 10 | 100.0% |
| | Nonpolyp | 10 | 10 | 100.0% |

Sleep dysfunction or poor sleep is associated with decreased quality of life and is a common complaint of approximately 60%-75% of individuals with CRS compared to 8%-18% of the general population.¹⁷ To evaluate the treatment effect of LYR-210 on sleep, the sleep dysfunction domain derived from the SNOT-22 was analyzed. The sleep dysfunction domain measures include difficulty falling asleep, waking up at night, lack of a good night's sleep, waking up tired, and fatigue. 18 LYR-210 (7500 μg) improved symptoms of sleep dysfunction throughout the 24-week treatment period with 90% of patients having achieved the MCID of 2.9 points for the SNOT-22 sleep dysfunction domain, as defined by Chowdhury et al.¹⁹, at week 24 (Table 3C).

TABLE 3C
Percent (%) Subjects Achieved MCID in SNOT-22
Sleep Dysfunction Domain. LYR-210 7500 μg.

| Time Point | N | RSP | PCT |
|---|---|---|---|
| Week 2 | 19 | 10 | 52.6% |
| Week 4 | 20 | 11 | 55.0% |
| Week 8 | 20 | 14 | 170.0% |
| Week 12 | 20 | 13 | 165.0% |
| Week 16 | 20 | 16 | 80.0% |
| Week 20 | 20 | 15 | 75.0% |
| Week 24 | 20 | 18 | 190.0% |

This discovery of a significant improvement for the SNOT-22 sleep dysfunction domain at week 24 was surprising based upon previous studies by others who pointed to a lack of such improvement using nonsurgical medical treatment. As one example, DeConde, et al., Int Forum Allergy Rhinol. 4(12): 972-979, 2014, showed that nonsurgical medical treatment did not significantly improve 2 general health-related QOL (Quality Of Life) domains, i.e., Sleep dysfunction and Psychological dysfunction, while surgical treatment with ongoing medical management did. In fact, patients choosing surgical treatment over nonsurgical medical treatment generally reported significantly higher pretreatment sleep dysfunction in addition to higher psychological dysfunction prior to surgery. Examples of medical treatment included oral, broad spectrum, or culture directed antibiotics; either topical nasal corticosteroid sprays or 5-day trials of systemic steroid therapy.

Moreover, DeConde, et al., reported there is a direct relationship between a worse baseline SNOT-22 score with a higher probability of a patient electing surgery. Metrics of baseline symptom severity appeared more effective at predicting treatment modality selection by a patient than a variety of other measures including: personality traits, risk aversion, degree of social support, economic factors and the patient-physician relationship. DeConde, et al., Int Forum Allergy Rhinol. 4(12): 972-979, 2014.

In fact, Endoscopic sinus surgery (ESS) was shown to be a more effective intervention treatment across all SNOT-22 categories than continued medical therapy, but was also found to show a differential effect across certain domains. DeConde, et al., Int Forum Allergy Rhinol. 4(12): 972-979,2014. Factor analysis of the SNOT-22 survey identified 5 distinct domains that are differentially impacted by endoscopic sinus surgery (ESS) vs. medical treatment. The first 2 domains were described above as being improved by surgery but not medical treatment (i.e., sleep dysfunction and psychological dysfunction). In contrast, 3 sinus-specific symptom domains (i.e., Rhinologic, Extra-rhinologic and Ear/facial symptoms) improved with either of these treatments but with caveats. Although significant differential mean improvement across all domain scores within both ESS and nonLYR-210 medical treatments was reported, the greatest improvements were in the group electing and undergoing sinus surgery.

Even though both surgical and medical treatment modalities resulted in improvement across all domains, subjects electing surgical interventions experienced greater relative improvement. That said, although subjects undergoing ESS experience greater gains than medical management across all domains; these gains were smallest in the sleep dysfunction and psychological dysfunction domains. DeConde, et al., Int Forum Allergy Rhinol. 4(12): 972-979, Published online 2014. Thus, patients improve in disease-specific quality-of-life (QOL) scores to a greater degree with surgical intervention than with medical management.

Emboldened by the surprising discovery that sleep dysfunction showed a statistically significant improvement of SNOT-22 scores from patients treated with a nonsurgical LYR-210 medical treatment as shown herein, the following study was done to see what additional domains might be improved using nonsurgical medical treatment using LYR-210 (7500 μg) over a nontreated control patient group.

Additionally, the use of patient reported outcomes measures (PROM), e.g., SNOT-22, to track clinical improvement leads to a problem of how to analyze these more abstract values of patient self-reporting in a clinically-relevant context, i.e., for use as a guide for one or more methods of: measuring the effects over time of LYR-210 medical treatment; measuring whether treatment outcome of LYR-210 is similar to outcomes of surgical intervention; etc. In some embodiments, methods for measuring treatment outcome are comparative LYR-210 medical treatments, i.e., between 2500 iug vs. 7500 μg. In some embodiments, methods for measuring treatment outcome are comparative LYR-210 medical treatments to control or sham patients. In some embodiments, methods for measuring treatment outcome of LYR-210 are used for suggesting a treatment type or change therapy (treatment).

Therefore, although tests like SNOT-22 were validated in numerous publications as reliable measures of disease at a particular time point, this validation process offers little guidance with how to interpret changes in scores over time. Thus, one additional objective herein is to use previously established thresholds by which a change in score, e.g., CFBL, is reflective of a true change in health status; and for use in outcomes research. This threshold is commonly known as the Minimal Clinically Important Change Score (MCID), see, for example, Cook. “Clinimetrics corner: the minimal clinically important change score (MCID): a necessary pretense.” Journal of Manual & Manipulative Therapy 16, no. 4: 82E-83E (2008).

MCID of SNOT-22 total and SNOT-22 (sub)domains were computed as mean MCID values for both SNOT-22 total and SNOT-22 domain scores before and after ESS, see, Chowdhury, et al., Int Forum Allergy Rhinol. 7(12): 1149-1155, 2017. As one example, Chowdhury, et al., suggested that for treatment outcomes research into CRS, 8.9 is generally accepted as the MCID threshold for improvement following sinus surgery however further suggested the use of 9.0 instead for ESS treated CRS patients.

Thus, as shown herein, patients with chronic rhinosinusitis were also evaluated (see bar graphs and Tables) using a predetermined MCID as provided for CRS surgery treated patients in Hopkins, et al., Clinical Otolaryngology, 34, 447-454, 2009 and for ESS surgery treated CRS patients in Chowdhury, et al., Int Forum Allergy Rhinol. 7(12): 1149-1155, 2017. One MCID unit of change for distribution-based methods is a mean MCID of the SNOT-22 total score or SNOT-22 domain score. A mean MCID of the SNOT-22 total score used herein was approximately 9.0. The average MCID for the rhinologic domain, extra-nasal rhinologic domain, ear/facial domain, psychological domain, and sleep domain score used were 3.8, 2.4, 3.2, 3.9, and 2.9, respectively. Albeit computed for surgical outcomes, these threshold values were used for measuring results of LYR-210 medical treatments, see Table 9A, below.

Further, as used herein, a minimal clinically important difference (MCID) refers to defining a threshold value by which a statistically significant result may also be thought to offer a clinically meaningful result. As one example, SNOT-22 results for psychological dysfunction for LYR-210 7500 μg at week 24 show significant statistical differences from baseline, along with a MCID that appears to be at least one 3.9 units of change from control. Further, 90% of patients achieved at least on MCID at week 24, See, Table 9A and Table 9B.

Although a MCID appears to be approaching 3.9, observed at weeks 4 and 8 in Table 9B, there is no significant statistical difference from control. Over that same time period, the percentage of patients achieving at least one MCID rose from around 65% to 75%, as shown in Table 9B. When patients are separated in polyp vs. nonpolyp groups, patients with nasal polyps show 80-90% and without show 70-90% response to LY-210 7500 μg as improvement of a MCID in sleep dysfunction, at weeks 12-24. See, Table 9C.

Therefore, in general, results from weeks 12-24 are considered clinically meaningful while values at weeks 4-8 are showing improvement reaching clinical MCID by around week 12. In another example, SNOT-22 results for sleep dysfunction for LYR-210 7500 μg at weeks 8-24 show statistical differences of improvement while at least approximately 65-90% of patients have MCID of at least 2.9 from control. See, Table 9A. When patients are separated in polyp vs. nonpolyp groups, patients with nasal polyps show 80-100% response to LYR-210 7500 μg as improvement of a MCID in sleep dysfunction, also at weeks 12-24. See, Table 9B.

In summary, this analysis supports the surprising results that LYR-210 7500 μg treatment improves sleep dysfunction and extends these finding to the surprise of a medical nonsurgical treatment for improvement of psychological dysfunction.

A. Impact of Long-Acting Implantable Corticosteroid Matrices on the SNOT-22 (Sub)Domains in Patients with Chronic Rhinosinusitis: Results from the LANTERN Study.

LYR-210 is an implantable matrix designed to release mometasone furoate for 24 weeks to inflamed sinonasal mucosa in chronic rhinosinusitis (CRS) patients. In the LANTERN study, LYR-210 demonstrated dose-dependent and significant improvement in the 22-item sinonasal outcome test (SNOT-22) total score compared to control, with all LYR-210 (7500 μg)-treated patients achieving the 8.9-point minimal clinically important difference (MCID) at week 24. The changes from baseline and proportion of patients achieving MCID at week 24 in each SNOT-22 (sub)domain from the LANTERN study were evaluated. Lower (more negative) CFBL scores in the SNOT-22 survey items suggest better patient functioning and improved symptoms. SNOT-22 refers to a patient-reported outcomes measurement used for providing a group average which in turn may be used to determine whether there is a Minimal Clinically Important Change Score (MCID). MCID refers to a change in score, e.g., a predetermined CFBL score, that might indicate changing the management of patients in the group. Cook. “Clinimetrics corner: the minimal clinically important change score (MCID): a necessary pretense.” Journal of Manual & Manipulative Therapy 16, no. 4: 82E-83E (2008).

Methods: Surgically naïve adults with moderate-to-severe CRS who failed previous medical management enrolled in a multicenter, randomized, controlled, dose-ranging (LANTERN) study. Twenty-three patients underwent a sham-procedure (control); and 21 or 23 patients received bilateral administration of LYR-210 (7500 μg) or LYR-210 (2500 μg), respectively. MOD values for the rhinologic, extranasal rhinologic, ear/facial, psychological, and sleep (dysfunction) domain scores are 3.8, 2.4, 3.2, 3.9, and 2.9, respectively (Chowdhury et al. 2017). Mean CFBL and percent patients achieving MOD in each SNOT-22 subdomain were analyzed using an ANCOVA model and logistic regression model, respectively.
Results: LYR-210 demonstrated dose-dependent symptom improvement with LYR-210 (7500 μg) achieving statistical significance (p<0.05) in each SNOT-22 (sub)domain compared to control at week 24. More LYR-210 (7500 μg)-treated patients achieved the MCID compared to control in the rhinologic (90% vs. 65%), extranasal rhinologic (71% vs. 52%), ear/facial (80% vs. 48%), psychological (90% vs. 78%), and sleep (90% vs. 61%) domains at week 24. See, Tables 4A-B though 9A-C.
Tables 4A-B though 9A-C show LYR-210 demonstrated dose-dependent symptom improvement with LYR-210 (7500 μg) achieving statistical significance (p<0.05) in each SNOT-22 domain compared to control at week 24.

Tables show the percent of subjects that achieved the MCID (minimal clinically important difference) in SNOT-22 total or subdomain scores. Data was analyzed on the ITT (Intention-To-Treat) population, which included all subjects who underwent a successful treatment procedure and had at least one post-randomization efficacy assessment, with LOCF (Last Observation Carried Forward).

Tables 4A and 4B show exemplary MCID response in the SNOT-22 total scores. Data represents % of subjects who achieved the MCID of at least an 8.9-point decrease in the SNOT-22 total score as defined in Hopkins, et al., Clinical Otolaryngology. 34, 447-454, 2009.

TABLE 4A
Percent (%) Subjects Achieved MCID in SNOT-22 Total Score.

| Time Point | Treatment | N | RSP | PCT |
|---|---|---|---|---|
| Week 2 | Sham | 21 | 8 | 38.1% |
| | LYR-210 2500 μg | 19 | 12 | 63.2% |
| | LYR-210 7500 μg | 19 | 9 | 47.4% |
| Week 4 | Sham | 23 | 14 | 60.9% |
| | LYR-210 2500 μg | 23 | 15 | 65.2% |
| | LYR-210 7500 μg | 20 | 14 | 70.0% |
| Week 8 | Sham | 23 | 15 | 65.2% |
| | LYR-210 2500 μg | 23 | 14 | 60.9% |
| | LYR-210 7500 μg | 20 | 16 | 80.0% |
| Week 12 | Sham | 23 | 16 | 69.6% |
| | LYR-210 2500 μg | 23 | 14 | 60.9% |
| | LYR-210 7500 μg | 20 | 17 | 85.0% |
| Week 16 | Sham | 23 | 15 | 65.2% |
| | LYR-210 2500 μg | 23 | 16 | 69.6% |
| | LYR-210 7500 μg | 20 | 18 | 90.0% |
| Week 20 | Sham | 23 | 16 | 69.6% |
| | LYR-210 2500 μg | 23 | 17 | 73.9% |
| | LYR-210 7500 μg | 20 | 19 | 95.0% |
| Week 24 | Sham | 23 | 15 | 65.2% |
| | LYR-210 2500 μg | 23 | 16 | 69.6% |
| | LYR-210 7500 μg | 20 | 20 | 100.0% |

TABLE 4B
Percent (%) Subjects Achieved MCID in SNOT-22
Total Score - polyp vs. nonpolyp.

| Time Point | Polyp Status | Treatment | N | RSP | PCT |
|---|---|---|---|---|---|
| Week 2 | Polyp | Sham | 12 | 5 | 41.7% |
| | | LYR-210 7500 μg | 10 | 3 | 30.0% |
| | Nonpolyp | Sham | 9 | 3 | 33.3% |
| | | LYR-210 7500 μg | 9 | 6 | 66.7% |
| Week 4 | Polyp | Sham | 13 | 9 | 69.2% |
| | | LYR-210 7500 μg | 10 | 6 | 60.0% |
| | Nonpolyp | Sham | 10 | 5 | 50.0% |
| | | LYR-210 7500 μg | 10 | 8 | 80.0% |
| Week 8 | Polyp | Sham | 13 | 9 | 69.2% |
| | | LYR-210 7500 μg | 10 | 7 | 70.0% |
| | Nonpolyp | Sham | 10 | 6 | 60.0% |
| | | LYR-210 7500 μg | 10 | 9 | 90.0% |
| Week 12 | Polyp | Sham | 13 | 8 | 61.5% |
| | | LYR-210 7500 μg | 10 | 8 | 80.0% |
| | Nonpolyp | Sham | 10 | 8 | 80.0% |
| | | LYR-210 7500 μg | 10 | 9 | 90.0% |
| Week 16 | Polyp | Sham | 13 | 8 | 61.5% |
| | | LYR-210 7500 μg | 10 | 9 | 90.0% |
| | Nonpolyp | Sham | 10 | 7 | 70.0% |
| | | LYR-210 7500 μg | 10 | 9 | 90.0% |
| Week 20 | Polyp | Sham | 13 | 8 | 61.5% |
| | | LYR-210 7500 μg | 10 | 9 | 90.0% |
| | Nonpolyp | Sham | 10 | 8 | 80.0% |
| | | LYR-210 7500 μg | 10 | 10 | 100.0% |
| Week 24 | Polyp | Sham | 13 | 8 | 61.5% |
| | | LYR-210 7500 μg | 10 | 10 | 100.0% |
| | Nonpolyp | Sham | 10 | 7 | 70.0% |
| | | LYR-210 7500 μg | 10 | 10 | 100.0% |

Table 5A shows exemplary CFBL in SNOT-22 rhinologic domain scores.

TABLE 5A
Change from Baseline in SNOT-22 Rhinologic Symptoms.

| Time Point | Treatment | N | LSMEAN | STDERR |
|---|---|---|---|---|
| Week 2 | Sham | 21 | −2.2 | 1.43 |
| | LYR-210 2500 μg | 19 | −1.3 | 1.61 |
| | LYR-210 7500 μg | 19 | −0.7 | 1.64 |
| Week 4 | Sham | 23 | −4.3 | 1.55 |
| | LYR-210 2500 μg | 23 | −4.5 | 1.62 |
| | LYR-210 7500 μg | 20 | −5.0 | 1.75 |
| Week 8 | Sham | 23 | −5.4 | 1.71 |
| | LYR-210 2500 μg | 23 | −4.1 | 1.79 |
| | LYR-210 7500 μg | 20 | −7.6 | 1.94 |
| Week 12 | Sham | 23 | −6.6 | 1.53 |
| | LYR-210 2500 μg | 23 | −6.7 | 1.60 |
| | LYR-210 7500 μg | 20 | −7.0 | 1.73 |
| Week 16 | Sham | 23 | −8.2 | 1.51 |
| | LYR-210 2500 μg | 23 | −7.3 | 1.58 |
| | LYR-210 7500 μg | 20 | −10.0 | 1.71 |
| Week 20 | Sham | 23 | −7.8 | 1.44 |
| | LYR-210 2500 μg | 23 | −8.6 | 1.51 |
| | LYR-210 7500 μg | 20 | −11.4 | 1.63 |
| Week 24 | Sham | 23 | −6.3 | 1.43 |
| | LYR-210 2500 μg | 23 | −6.3 | 1.50 |
| | LYR-210 7500 μg | 20 | −10.0 | 1.62 |

Tables 5B and 5C show exemplary MCID response in the SNOT-22 rhinologic domain scores. Data represents % of subjects who achieved the MCID of at least a 3.8-point decrease in the SNOT-22 Rhinologic domain score, as defined in Chowdhury, et al., Int Forum Allergy Rhinol. 7(12): 1149-1155, 2017.

TABLE 5B
Percent (%) Subjects Achieved MCID in SNOT-22
Rhinologic Domain.

| Time Point | Treatment | N | RSP | PCT |
|---|---|---|---|---|
| Week 2 | Sham | 21 | 7 | 33.3% |
| | LYR-210 2500 μg | 19 | 7 | 36.8% |
| | LYR-210 7500 μg | 19 | 5 | 26.3% |
| Week 4 | Sham | 23 | 11 | 47.8% |
| | LYR-210 2500 μg | 23 | 10 | 43.5% |
| | LYR-210 7500 μg | 20 | 11 | 55.0% |
| Week 8 | Sham | 23 | 13 | 56.5% |
| | LYR-210 2500 μg | 23 | 10 | 43.5% |
| | LYR-210 7500 μg | 20 | 13 | 65.0% |
| Week 12 | Sham | 23 | 15 | 65.2% |
| | LYR-210 2500 μg | 23 | 13 | 56.5% |
| | LYR-210 7500 μg | 20 | 13 | 65.0% |
| Week 16 | Sham | 23 | 15 | 65.2% |
| | LYR-210 2500 μg | 23 | 15 | 65.2% |
| | LYR-210 7500 μg | 20 | 16 | 80.0% |
| Week 20 | Sham | 23 | 13 | 56.5% |
| | LYR-210 2500 μg | 23 | 15 | 65.2% |
| | LYR-210 7500 μg | 20 | 16 | 80.0% |
| Week 24 | Sham | 23 | 15 | 65.2% |
| | LYR-210 2500 μg | 23 | 12 | 52.2% |
| | LYR-210 7500 μg | 20 | 18 | 90.0% |

TABLE 5C
Percent (%) Subjects Achieved MCID in SNOT-22
Rhinologic Domain - polyp vs. nonpolyp.

| Time Point | Polyp Status | Treatment | N | RSP | PCT |
|---|---|---|---|---|---|
| Week 2 | Polyp | Sham | 12 | 4 | 33.3% |
| | | LYR-210 7500 μg | 10 | 3 | 30.0% |
| | Nonpolyp | Sham | 9 | 3 | 33.3% |
| | | LYR-210 7500 μg | 9 | 2 | 22.2% |
| Week 4 | Polyp | Sham | 13 | 7 | 53.8% |
| | | LYR-210 7500 μg | 10 | 4 | 40.0% |
| | Nonpolyp | Sham | 10 | 4 | 40.0% |
| | | LYR-210 7500 μg | 10 | 7 | 70.0% |
| Week 8 | Polyp | Sham | 13 | 7 | 53.8% |
| | | LYR-210 7500 μg | 10 | 6 | 60.0% |
| | Nonpolyp | Sham | 10 | 6 | 60.0% |
| | | LYR-210 7500 μg | 10 | 7 | 70.0% |
| Week 12 | Polyp | Sham | 13 | 9 | 69.2% |
| | | LYR-210 7500 μg | 10 | 7 | 70.0% |
| | Nonpolyp | Sham | 10 | 6 | 60.0% |
| | | LYR-210 7500 μg | 10 | 6 | 60.0% |
| Week 16 | Polyp | Sham | 13 | 8 | 61.5% |
| | | LYR-210 7500 μg | 10 | 8 | 80.0% |
| | Nonpolyp | Sham | 10 | 7 | 70.0% |
| | | LYR-210 7500 μg | 10 | 8 | 80.0% |
| Week 20 | Polyp | Sham | 13 | 7 | 53.8% |
| | | LYR-210 7500 μg | 10 | 6 | 60.0% |
| | Nonpolyp | Sham | 10 | 6 | 60.0% |
| | | LYR-210 7500 μg | 10 | 10 | 100.0% |
| Week 24 | Polyp | Sham | 13 | 8 | 61.5% |
| | | LYR-210 7500 μg | 10 | 8 | 80.0% |
| | Nonpolyp | Sham | 10 | 7 | 70.0% |
| | | LYR-210 7500 μg | 10 | 10 | 100.0% |

Table 6A shows exemplary CBFL in SNOT-22 extranasal rhinologic domain scores.

TABLE 6A
Change from Baseline in SNOT-22 Extra-Nasal
Rhinologic Symptoms.

| Time Point | Treatment | N | LSMEAN | STDERR |
|---|---|---|---|---|
| Week 2 | Sham | 21 | −1.7 | 0.79 |
| | LYR-210 2500 μg | 19 | −1.0 | 0.90 |
| | LYR-210 7500 μg | 19 | −1.5 | 0.90 |
| Week 4 | Sham | 23 | −2.9 | 0.86 |
| | LYR-210 2500 μg | 23 | −2.8 | 0.90 |
| | LYR-210 7500 μg | 20 | −3.7 | 0.96 |
| Week 8 | Sham | 23 | −2.9 | 0.79 |
| | LYR-210 2500 μg | 23 | −2.7 | 0.83 |
| | LYR-210 7500 μg | 20 | −4.3 | 0.89 |
| Week 12 | Sham | 23 | −3.2 | 0.87 |
| | LYR-210 2500 μg | 23 | −3.1 | 0.91 |
| | LYR-210 7500 μg | 20 | −3.7 | 0.98 |
| Week 16 | Sham | 23 | −3.6 | 0.84 |
| | LYR-210 2500 μg | 23 | −3.7 | 0.87 |
| | LYR-210 7500 μg | 20 | −5.1 | 0.94 |
| Week 20 | Sham | 23 | −3.7 | 0.73 |
| | LYR-210 2500 μg | 23 | −4.4 | 0.76 |
| | LYR-210 7500 μg | 20 | −6.0 | 0.82 |
| Week 24 | Sham | 23 | −3.1 | 0.72 |
| | LYR-210 2500 μg | 23 | −3.7 | 0.75 |
| | LYR-210 7500 μg | 20 | −5.2 | 0.81 |

Tables 6B and 6C show exemplary MCID response in the SNOT-22 extranasal rhinologic domain scores. Data represents % of subjects who achived the MCID of at least a 2.4-point decrease in the SNOT-22 extranasal rhinologic domain score, as defined in Chowdhury, et al., Int Forum Allergy Rhinol. 7(12): 1149-1155, 2017.

TABLE 6B
Percent (%) Subjects Achieved MCID in SNOT-22
Extra-Nasal Rhinologic Domain.

| Time Point | Treatment | N | RSP | PCT |
|---|---|---|---|---|
| Week 2 | Sham | 21 | 6 | 28.6% |
| | LYR-210 2500 μg | 19 | 7 | 36.8% |
| | LYR-210 7500 μg | 19 | 6 | 31.6% |
| Week 4 | Sham | 23 | 9 | 39.1% |
| | LYR-210 2500 μg | 23 | 10 | 43.5% |
| | LYR-210 7500 μg | 20 | 10 | 50.0% |
| Week 8 | Sham | 23 | 11 | 47.8% |
| | LYR-210 2500 μg | 23 | 13 | 56.5% |
| | LYR-210 7500 μg | 20 | 11 | 55.0% |
| Week 12 | Sham | 23 | 11 | 47.8% |
| | LYR-210 2500 μg | 23 | 13 | 56.5% |
| | LYR-210 7500 μg | 20 | 10 | 50.0% |
| Week 16 | Sham | 23 | 11 | 47.8% |
| | LYR-210 2500 μg | 23 | 13 | 56.5% |
| | LYR-210 7500 μg | 20 | 13 | 65.0% |
| Week 20 | Sham | 23 | 11 | 47.8% |
| | LYR-210 2500 μg | 23 | 16 | 69.6% |
| | LYR-210 7500 μg | 20 | 14 | 70.0% |
| Week 24 | Sham | 23 | 12 | 52.2% |
| | LYR-210 2500 μg | 23 | 14 | 60.9% |
| | LYR-210 7500 μg | 20 | 14 | 70.0% |

TABLE 6C
Percent (%) Subjects Achieved MCID in SNOT-22
Extra-Nasal Rhinologic Domain - polyp vs. nonpolyp.

| Time Point | Polyp Status | Treatment | N | RSP | PCT |
|---|---|---|---|---|---|
| Week 2 | Polyp | Sham | 12 | 3 | 25.0% |
| | | LYR-210 7500 μg | 10 | 3 | 30.0% |
| | Nonpolyp | Sham | 9 | 3 | 33.3% |
| | | LYR-210 7500 μg | 9 | 3 | 33.3% |
| Week 4 | Polyp | Sham | 13 | 6 | 46.2% |
| | | LYR-210 7500 μg | 10 | 5 | 50.0% |
| | Nonpolyp | Sham | 10 | 3 | 30.0% |
| | | LYR-210 7500 μg | 10 | 5 | 50.0% |
| Week 8 | Polyp | Sham | 13 | 7 | 53.8% |
| | | LYR-210 7500 μg | 10 | 5 | 50.0% |
| | Nonpolyp | Sham | 10 | 4 | 40.0% |
| | | LYR-210 7500 μg | 10 | 6 | 60.0% |
| Week 12 | Polyp | Sham | 13 | 6 | 46.2% |
| | | LYR-210 7500 μg | 10 | 3 | 30.0% |
| | Nonpolyp | Sham | 10 | 5 | 50.0% |
| | | LYR-210 7500 μg | 10 | 7 | 70.0% |
| Week 16 | Polyp | Sham | 13 | 6 | 46.2% |
| | | LYR-210 7500 μg | 10 | 6 | 60.0% |
| | Nonpolyp | Sham | 10 | 5 | 50.0% |
| | | LYR-210 7500 μg | 10 | 7 | 70.0% |
| Week 20 | Polyp | Sham | 13 | 5 | 38.5% |
| | | LYR-210 7500 μg | 10 | 4 | 40.0% |
| | Nonpolyp | Sham | 10 | 6 | 60.0% |
| | | LYR-210 7500 μg | 10 | 10 | 100.0% |
| Week 24 | Polyp | Sham | 13 | 6 | 46.2% |
| | | LYR-210 7500 μg | 10 | 5 | 50.0% |
| | Nonpolyp | Sham | 10 | 6 | 60.0% |
| | | LYR-210 7500 μg | 10 | 9 | 90.0% |

Table 7A shows exemplary CFBL in SNOT-22 ear/facial domain scores.

TABLE 7A
Change from Baseline in SNOT-22 Ear/Facial Symptoms.

| Time Point | Treatment | N | LSMEAN | STDERR |
|---|---|---|---|---|
| Week 2 | Sham | 21 | −1.0 | 1.18 |
| | LYR-210 2500 μg | 19 | −2.2 | 1.32 |
| | LYR-210 7500 μg | 19 | −0.9 | 1.37 |
| Week 4 | Sham | 23 | −2.7 | 1.27 |
| | LYR-210 2500 μg | 23 | −3.6 | 1.33 |
| | LYR-210 7500 μg | 20 | −4.2 | 1.46 |
| Week 8 | Sham | 23 | −3.9 | 1.35 |
| | LYR-210 2500 μg | 23 | −3.9 | 1.42 |
| | LYR-210 7500 μg | 20 | −6.4 | 1.56 |
| Week 12 | Sham | 23 | −3.9 | 1.28 |
| | LYR-210 2500 μg | 23 | −3.8 | 1.35 |
| | LYR-210 7500 μg | 20 | −5.9 | 1.48 |
| Week 16 | Sham | 23 | −4.9 | 1.20 |
| | LYR-210 2500 μg | 23 | −6.3 | 1.26 |
| | LYR-210 7500 μg | 20 | −8.5 | 1.39 |
| Week 20 | Sham | 23 | −4.1 | 1.24 |
| | LYR-210 2500 μg | 23 | −7.4 | 1.30 |
| | LYR-210 7500 μg | 20 | −8.1 | 1.43 |
| Week 24 | Sham | 23 | −3.8 | 1.25 |
| | LYR-210 2500 μg | 23 | −6.1 | 1.31 |
| | LYR-210 7500 μg | 20 | −8.0 | 1.44 |

Tables 7B and 7C show exemplary MCID response in the SNOT-22 ear/facial domain scores. Data represents % of subjects who achieved the MCID of at least a 3.2-point decrease in the SNOT-22 Ear-Facial domain score, as defined in Chowdhury, et al., Int Forum Allergy Rhinol. 7(12): 1149-1155, 2017.

TABLE 7B
Percent (%) Subjects Achieved MCID in SNOT-22 Ear/Facial
Domain.

| Time Point | Treatment | N | RSP | PCT |
|---|---|---|---|---|
| Week 2 | Sham | 21 | 7 | 33.3% |
| | LYR-210 2500 μg | 19 | 8 | 42.1% |
| | LYR-210 7500 μg | 19 | 6 | 31.6% |
| Week 4 | Sham | 23 | 10 | 43.5% |
| | LYR-210 2500 μg | 23 | 10 | 43.5% |
| | LYR-210 7500 μg | 20 | 12 | 60.0% |
| Week 8 | Sham | 23 | 14 | 60.9% |
| | LYR-210 2500 μg | 23 | 10 | 43.5% |
| | LYR-210 7500 μg | 20 | 14 | 70.0% |
| Week 12 | Sham | 23 | 14 | 60.9% |
| | LYR-210 2500 μg | 23 | 13 | 156.5% |
| | LYR-210 7500 μg | 20 | 13 | 65.0% |
| Week 16 | Sham | 23 | 13 | 56.5% |
| | LYR-210 2500 μg | 23 | 13 | 56.5% |
| | LYR-210 7500 μg | 20 | 14 | 70.0% |
| Week 20 | Sham | 23 | 12 | 52.2% |
| | LYR-210 2500 μg | 23 | 14 | 60.9% |
| | LYR-210 7500 μg | 20 | 15 | 75.0% |
| Week 24 | Sham | 23 | 11 | 47.8% |
| | LYR-210 2500 μg | 23 | 14 | 60.9% |
| | LYR-210 7500 μg | 20 | 16 | 80.0% |

TABLE 7C
Percent (%) Subjects Achieved MCID in SNOT-22 Ear/Facial
Domain-polyp vs. nonpolyp.

| Time Point | Polyp Status | Treatment | N | RSP | PCT |
|---|---|---|---|---|---|
| Week 2 | Polyp | Sham | 12 | 5 | 41.7% |
| | | LYR-210 7500 μg | 10 | 3 | 30.0% |
| | Nonpolyp | Sham | 9 | 2 | 22.2% |
| | | LYR-210 7500 μg | 9 | 3 | 33.3% |
| Week 4 | Polyp | Sham | 13 | 6 | 46.2% |
| | | LYR-210 7500 μg | 10 | 5 | 50.0% |
| | Nonpolyp | Sham | 10 | 4 | 40.0% |
| | | LYR-210 7500 μg | 10 | 7 | 70.0% |
| Week 8 | Polyp | Sham | 13 | 8 | 61.5% |
| | | LYR-210 7500 μg | 10 | 6 | 60.0% |
| | Nonpolyp | Sham | 10 | 6 | 60.0% |
| | | LYR-210 7500 μg | 10 | 8 | 80.0% |
| Week 12 | Polyp | Sham | 13 | 8 | 61.5% |
| | | LYR-210 7500 μg | 10 | 4 | 40.0% |
| | Nonpolyp | Sham | 10 | 6 | 60.0% |
| | | LYR-210 7500 μg | 10 | 9 | 90.0% |
| Week 16 | Polyp | Sham | 13 | 8 | 61.5% |
| | | LYR-210 7500 μg | 10 | 6 | 60.0% |
| | Nonpolyp | Sham | 10 | 5 | 50.0% |
| | | LYR-210 7500 μg | 10 | 8 | 80.0% |
| Week 20 | Polyp | Sham | 13 | 8 | 61.5% |
| | | LYR-210 7500 μg | 10 | 5 | 50.0% |
| | Nonpolyp | Sham | 10 | 4 | 40.0% |
| | | LYR-210 7500 μg | 10 | 10 | 100.0% |
| Week 24 | Polyp | Sham | 13 | 8 | 61.5% |
| | | LYR-210 7500 μg | 10 | 6 | 60.0% |
| | Nonpolyp | Sham | 10 | 3 | 30.0% |
| | | LYR-210 7500 μg | 10 | 10 | 100.0% |

Table 8A shows exemplary CHIL in SNOT-22 psychological dysfunction domain scores.

TABLE 8A
Change from Baseline in SNOT-22 Psychological Dysfunction.

| Time Point | Treatment | N | LSMEAN | STDERR |
|---|---|---|---|---|
| Week 2 | Sham | 21 | −5.6 | 2.11 |
| | LYR-210 2500 μg | 19 | −5.8 | 2.39 |
| | LYR-210 7500 μg | 19 | −5.0 | 2.37 |
| Week 4 | Sham | 23 | −5.9 | 1.91 |
| | LYR-210 2500 μg | 23 | −8.5 | 1.99 |
| | LYR-210 7500 μg | 20 | −9.6 | 2.14 |
| Week 8 | Sham | 23 | −5.7 | 2.08 |
| | LYR-210 2500 μg | 23 | −6.7 | 2.17 |
| | LYR-210 7500 μg | 20 | −9.7 | 2.32 |
| Week 12 | Sham | 23 | −8.7 | 1.93 |
| | LYR-210 2500 μg | 23 | −10.3 | 2.01 |
| | LYR-210 7500 μg | 20 | −13.2 | 2.16 |
| Week 16 | Sham | 23 | −8.4 | 2.00 |
| | LYR-210 2500 μg | 23 | −11.2 | 2.09 |
| | LYR-210 7500 μg | 20 | −14.6 | 2.24 |
| Week 20 | Sham | 23 | −9.2 | 1.85 |
| | LYR-210 2500 μg | 23 | −12.9 | 1.93 |
| | LYR-210 7500 μg | 20 | −15.5 | 2.07 |
| Week 24 | Sham | 23 | −8.6 | 1.98 |
| | LYR-210 2500 μg | 23 | −12.1 | 2.06 |
| | LYR-210 7500 μg | 20 | −15.2 | 2.21 |

Tables 8B and 8C shows exemplary MCID response in the SNOT-22 psychological dysfunction domain scores. Data represents % of subjects who achieved the MCID of at least a 3.9-point decrease in the SNOT-22 Psychological domain score, as defined in Chowdhury, et al., Int Forum Allergy Rhinol. 7(12): 1149-1155, 2017.

TABLE 8B
Percent (%) Subjects Achieved MCID in SNOT-22
Psychological Domain.

| Time Point | Treatment | N | RSP | PCT |
|---|---|---|---|---|
| Week 2 | Sham | 21 | 12 | 57.1% |
| | LYR-210 2500 μg | 19 | 9 | 47.4% |
| | LYR-210 7500 μg | 19 | 9 | 47.4% |
| Week 4 | Sham | 23 | 12 | 52.2% |
| | LYR-210 2500 μg | 23 | 14 | 60.9% |
| | LYR-210 7500 μg | 20 | 13 | 65.0% |
| Week 8 | Sham | 23 | 14 | 60.9% |
| | LYR-210 2500 μg | 23 | 12 | 52.2% |
| | LYR-210 7500 μg | 20 | 15 | 75.0% |
| Week 12 | Sham | 23 | 17 | 73.9% |
| | LYR-210 2500 μg | 23 | 14 | 60.9% |
| | LYR-210 7500 μg | 20 | 15 | 75.0% |
| Week 16 | Sham | 23 | 16 | 69.6% |
| | LYR-210 2500 μg | 23 | 17 | 73.9% |
| | LYR-210 7500 μg | 20 | 15 | 75.0% |
| Week 20 | Sham | 23 | 17 | 73.9% |
| | LYR-210 2500 μg | 23 | 16 | 69.6% |
| | LYR-210 7500 μg | 20 | 17 | 85.0% |
| Week 24 | Sham | 23 | 18 | 78.3% |
| | LYR-210 2500 μg | 23 | 16 | 69.6% |
| | LYR-210 7500 μg | 20 | 18 | 90.0% |

TABLE 8C
Percent (%) Subjects Achieved MCID in SNOT-22 Psychological
Domain-polyp vs. nonpolyp.

| Time Point | Polyp Status | Treatment | N | RSP | PCT |
|---|---|---|---|---|---|
| Week 2 | Polyp | Sham | 12 | 7 | 58.3% |
| | | LYR-210 7500 μg | 10 | 4 | 40.0% |
| | Nonpolyp | Sham | 9 | 5 | 55.6% |
| | | LYR-210 7500 μg | 9 | 5 | 55.6% |
| Week 4 | Polyp | Sham | 13 | 7 | 53.8% |
| | | LYR-210 7500 μg | 10 | 5 | 50.0% |
| | Nonpolyp | Sham | 10 | 5 | 50.0% |
| | | LYR-210 7500 μg | 10 | 8 | 80.0% |
| Week 8 | Polyp | Sham | 13 | 8 | 61.5% |
| | | LYR-210 7500 μg | 10 | 7 | 70.0% |
| | Nonpolyp | Sham | 10 | 6 | 60.0% |
| | | LYR-210 7500 μg | 10 | 8 | 80.0% |
| Week 12 | Polyp | Sham | 13 | 8 | 61.5% |
| | | LYR-210 7500 μg | 10 | 8 | 80.0% |
| | Nonpolyp | Sham | 10 | 9 | 90.0% |
| | | LYR-210 7500 μg | 10 | 7 | 70.0% |
| Week 16 | Polyp | Sham | 13 | 8 | 61.5% |
| | | LYR-210 7500 μg | 10 | 8 | 80.0% |
| | Nonpolyp | Sham | 10 | 8 | 80.0% |
| | | LYR-210 7500 μg | 10 | 7 | 70.0% |
| Week 20 | Polyp | Sham | 13 | 9 | 69.2% |
| | | LYR-210 7500 μg | 10 | 8 | 80.0% |
| | Nonpolyp | Sham | 10 | 8 | 80.0% |
| | | LYR-210 7500 μg | 10 | 9 | 90.0% |
| Week 24 | Polyp | Sham | 13 | 9 | 69.2% |
| | | LYR-210 7500 μg | 10 | 9 | 90.0% |
| | Nonpolyp | Sham | 10 | 9 | 90.0% |
| | | LYR-210 7500 μg | 10 | 9 | 90.0% |

Table 9A shows exemplary CI-13L in SNOT-22 sleep dysfunction domain scores.

TABLE 9A
Change from Baseline in SNOT-22 Sleep Dysfunction.

| Time Point | Treatment | N | LSMEAN | STDERR |
|---|---|---|---|---|
| Week 2 | Sham | 21 | −1.9 | 1.52 |
| | LYR-210 2500 μg | 19 | −3.3 | 1.70 |
| | LYR-210 7500 μg | 19 | −2.9 | 1.70 |
| Week 4 | Sham | 23 | −2.5 | 1.55 |
| | LYR-210 2500 μg | 23 | −4.8 | 1.61 |
| | LYR-210 7500 μg | 20 | −5.6 | 1.72 |
| Week 8 | Sham | 23 | −2.3 | 1.64 |
| | LYR-210 2500 μg | 23 | −3.6 | 1.70 |
| | LYR-210 7500 μg | 20 | −6.4 | 1.82 |
| Week 12 | Sham | 23 | −4.3 | 1.58 |
| | LYR-210 2500 μg | 23 | −6.3 | 1.64 |
| | LYR-210 7500 μg | 20 | −8.0 | 1.75 |
| Week 16 | Sham | 23 | −4.2 | 1.50 |
| | LYR-210 2500 μg | 23 | −6.8 | 1.56 |
| | LYR-210 7500 μg | 20 | −9.0 | 1.66 |
| Week 20 | Sham | 23 | −5.8 | 1.47 |
| | LYR-210 2500 μg | 23 | −9.2 | 1.53 |
| | LYR-210 7500 μg | 20 | −11.0 | 1.63 |
| Week 24 | Sham | 23 | −4.1 | 1.60 |
| | LYR-210 2500 μg | 23 | −7.5 | 1.66 |
| | LYR-210 7500 μg | 20 | −10.3 | 1.77 |

Tables 9B and 9C shows exemplary MCID response in the SNOT-22 sleep dysfunction domain scores. Data represents % of subjects who achieved the MCID of at least a 2.9-point decrease in
The SNOT-22 Sleep Dysfunction domain score, as defined in Chowdhury, et al., Int Forum Allergy

### Rhinol. 7(12): 1149-1155,2017.

TABLE 9B
Percent (%) Subjects Achieved MCID in SNOT-22 Sleep
Dysfunction Domain.

| Time Point | Treatment | N | RSP | PCT |
|---|---|---|---|---|
| Week 2 | Sham | 21 | 9 | 42.9% |
| | LYR-210 2500 μg | 19 | 11 | 57.9% |
| | LYR-210 7500 μg | 19 | 10 | 52.6% |
| Week 4 | Sham | 23 | 12 | 52.2% |
| | LYR-210 2500 μg | 23 | 15 | 65.2% |
| | LYR-210 7500 μg | 20 | 11 | 55.0% |
| Week 8 | Sham | 23 | 13 | 56.5% |
| | LYR-210 2500 μg | 23 | 11 | 47.8% |
| | LYR-210 7500 μg | 20 | 14 | 70.0% |
| Week 12 | Sham | 23 | 15 | 65.2% |
| | LYR-210 2500 μg | 23 | 15 | 65.2% |
| | LYR-210 7500 μg | 20 | 13 | 65.0% |
| Week 16 | Sham | 23 | 15 | 65.2% |
| | LYR-210 2500 μg | 23 | 15 | 65.2% |
| | LYR-210 7500 μg | 20 | 16 | 80.0% |
| Week 20 | Sham | 23 | 15 | 65.2% |
| | LYR-210 2500 μg | 23 | 16 | 69.6% |
| | LYR-210 7500 μg | 20 | 15 | 75.0% |
| Week 24 | Sham | 23 | 15 | 65.2% |
| | LYR-210 2500 μg | 23 | 15 | 65.2% |
| | LYR-210 7500 μg | 20 | 18 | 90.0% |

TABLE 9C
Percent (%) Subjects Achieved MCID in SNOT-22 Sleep
Dysfunction Domain-polyp vs. nonpolyp.

| Time Point | Polyp Status | Treatment | N | RSP | PCT |
|---|---|---|---|---|---|
| Week 2 | Polyp | Sham | 12 | 5 | 41.7% |
| | | LYR-210 7500 μg | 10 | 5 | 50.0% |
| | Nonpolyp | Sham | 9 | 4 | 44.4% |
| | | LYR-210 7500 μg | 9 | 5 | 55.6% |
| Week 4 | Polyp | Sham | 13 | 8 | 61.5% |
| | | LYR-210 7500 μg | 10 | 4 | 40.0% |
| | Nonpolyp | Sham | 10 | 4 | 40.0% |
| | | LYR-210 7500 μg | 10 | 7 | 70.0% |
| Week 8 | Polyp | Sham | 13 | 7 | 53.8% |
| | | LYR-210 7500 μg | 10 | 5 | 50.0% |
| | Nonpolyp | Sham | 10 | 6 | 60.0% |
| | | LYR-210 7500 μg | 10 | 9 | 90.0% |
| Week 12 | Polyp | Sham | 13 | 8 | 61.5% |
| | | LYR-210 7500 μg | 10 | 5 | 50.0% |
| | Nonpolyp | Sham | 10 | 7 | 70.0% |
| | | LYR-210 7500 μg | 10 | 8 | 80.0% |
| Week 16 | Polyp | Sham | 13 | 8 | 61.5% |
| | | LYR-210 7500 μg | 10 | 6 | 60.0% |
| | Nonpolyp | Sham | 10 | 7 | 70.0% |
| | | LYR-210 7500 μg | 10 | 10 | 100.0% |
| Week 20 | Polyp | Sham | 13 | 7 | 53.8% |
| | | LYR-210 7500 μg | 10 | 6 | 60.0% |
| | Nonpolyp | Sham | 10 | 8 | 80.0% |
| | | LYR-210 7500 μg | 10 | 9 | 90.0% |
| Week 24 | Polyp | Sham | 13 | 7 | 53.8% |
| | | LYR-210 7500 μg | 10 | 8 | 80.0% |
| | Nonpolyp | Sham | 10 | 8 | 80.0% |
| | | LYR-210 7500 μg | 10 | 10 | 100.0% |

Conclusions: LYR-210 demonstrated improvement in all SNOT-22 (sub)domains and may be a promising long-acting sinonasal treatment for surgically naive CRS patients.

Furthermore, when patients are surgical candidates, these improvements indicate that when such patients are treated with LYR-210 7500 μg they may no longer be considered as a candidate for ESS. Determining a minimally important difference provides a way to interpret scores in a clinical context and may help to improve patient selection of treatment or guide a treatment professional or a patient's choice for undergoing surgery or choosing a medical treatment such as LYR-210, 2500 μg or 7500 μg. In other words, if a patient mainly would like to improve sleep dysfunction, that patient may choose a LYR-210 7500 lig treatment.

In part because this surprising discovery of nonsurgical medical management with LYR-210 (2500 μg) and/or LYR-210 (7500 μg) improving quality of life similar to ESS treated patients as part of the 22-item SinoNasal Outcomes Test (SNOT-22), further studies were done in order to support this surprising finding for use in light of MCID values.

B. Quality of Life Evaluation Using the 36-Item Short form Health Survey in Chronic Rhinosinusitis Patients Treated with Long-Acting Implantable Corticosteroid Matrices.

The quality of life of the LANTERN study patients was evaluated using the 36-item short form health survey, version 2 (SF-36v2). LYR-210 is an implantable matrix intended to release mometasone furoate for 24 weeks to inflamed sinonasal mucosa in chronic rhinosinusitis (CRS) patients. In the randomized, controlled LANTERN study, LYR-210 (7500 μg) demonstrated clinically relevant symptom improvement and decreased ethmoid opacification and need for rescue medication at week 24.

Methods: Surgically naïve adults with moderate-to-severe CRS who failed previous medical management enrolled in a multicenter, randomized, controlled, dose-ranging study. Patients were randomized (1:1:1) to either bilateral administration of LYR-210 (2500 μg) (n=23) or LYR-210 (7500 μg) (n=21), or to sham-procedure controls (n=23). The SF-36v2 was completed by ePRO at baseline and week 24. The mental health component summary (MCS) and its (sub)domains (vitality, social functioning, role-emotional, mental health), and physical health component summary (PCS) and its (sub)domains (physical functioning, role-physical, bodily pain, general health) were analyzed as change from baseline. LYR-210 groups were compared to control using an ANCOVA model. P<0.05 was statistically significant.
Results: Both LYR-210 doses significantly improved the MCS score by >8 points over control at week 24. LYR-210 (7500 μg) achieved statistical significance in each of the 4 MCS domains (vitality, social functioning, role-emotional, and mental health). LYR-210 (2500 μg) and (7500 μg) numerically increased the PCS score compared to control at week 24, with LYR-210 (7500 μg) achieving statistical significance in 3 PCS domains (physical functioning, role-physical, and bodily pain). Each of the domain scores is oriented so that a higher score represents better health. See, Tables 10-11.
Tables 10-11 show a quality-of-life evaluation using the SF-36v2 health survey. Data were analyzed on the ITT population using an ANCOVA model. Missing data were not imputed. Data presented are least squares means. Both LYR-210 (2500 μg) and LYR-210 (7500 μg) significantly improved the mean mental component summary (MCS) score at week 24. LYR-210 (7500 μg) achieved statistical significance in each of the 4 mental component (MCS) domains (vitality, social functioning, role-emotional, and mental health). LYR-210 (2500 μg) and (7500 μg) numerically increased the physical component (PCS) score compared to control at week 24, with LYR-210 (7500 ttg) achieving statistical significance in 3 PCS domains (physical functioning, role-physical, and bodily pain). Each of the (sub)domain scores was oriented so that a higher score represents better health. * Indicates statistical significance (1-sided p<0.05 vs. control).
Tables 10A-10E shows exemplary CFBL in SF-36v2 scores for a mental health component summary (MCS) and the vitality, social functioning, role-emotional, and mental health scores.

TABLE 10A
Change from Baseline in SF-36 Mental Component Score.

| Time Point | Treatment | N | LSMEAN | STDERR |
|---|---|---|---|---|
| Week 24 | Sham | 10 | 1.7 | 2.99 |
| | LYR-210 2500 μg | 16 | 10.0 | 2.78 |
| | LYR-210 7500 μg | 14 | 10.2 | 2.89 |
| Week 48 | Sham | 10 | 7.7 | 3.94 |
| | LYR-210 2500 μg | 17 | 7.9 | 3.30 |
| | LYR-210 7500 μg | 17 | 7.7 | 3.30 |

TABLE 10B
Change from Baseline in SF-36 Vitality.

| Time Point | Treatment | N | LSMEAN | STDERR |
|---|---|---|---|---|
| Week 24 | Sham | 10 | 6.1 | 3.05 |
| | LYR-210 2500 μg | 16 | 9.9 | 2.82 |
| | LYR-210 7500 μg | 14 | 11.4 | 2.93 |
| Week 48 | Sham | 10 | 7.4 | 3.82 |
| | LYR-210 2500 μg | 17 | 9.2 | 3.20 |
| | LYR-210 7500 μg | 17 | 11.9 | 3.23 |

TABLE 10C
Change from Baseline in SF-36 Social Functioning.

| Time Point | Treatment | N | LSMEAN | STDERR |
|---|---|---|---|---|
| Week 24 | Sham | 10 | 2.2 | 2.95 |
| | LYR-210 2500 μg | 16 | 11.5 | 2.76 |
| | LYR-210 7500 μg | 14 | 11.7 | 2.88 |
| Week 48 | Sham | 10 | 8.3 | 4.43 |
| | LYR-210 2500 μg | 17 | 8.8 | 3.72 |
| | LYR-210 7500 μg | 17 | 10.4 | 3.77 |

TABLE 10D
Change from Baseline in SF-36 Role Emotional.

| Time Point | Treatment | N | LSMEAN | STDERR |
|---|---|---|---|---|
| Week 24 | Sham | 10 | 0.5 | 2.99 |
| | LYR-210 2500 μg | 16 | 8.0 | 2.78 |
| | LYR-210 7500 μg | 14 | 8.7 | 2.89 |
| Week 48 | Sham | 10 | 3.1 | 3.86 |
| | LYR-210 2500 μg | 17 | 5.0 | 3.25 |
| | LYR-210 7500 μg | 17 | 5.2 | 3.24 |

TABLE 10E
Change from Baseline in SF-36 Mental Health.

| Time Point | Treatment | N | LSMEAN | STDERR |
|---|---|---|---|---|
| Week 24 | Sham | 10 | 2.8 | 2.67 |
| | LYR-210 2500 μg | 16 | 9.8 | 2.47 |
| | LYR-210 7500 μg | 14 | 9.8 | 2.56 |
| Week 48 | Sham | 10 | 5.9 | 3.81 |
| | LYR-210 2500 μg | 17 | 6.5 | 3.19 |
| | LYR-210 7500 μg | 17 | 6.9 | 3.19 |

Tables 11A-11E shows exemplary CFBL in SF-36v2 scores for a physical health component summary (PCS) and physical functioning, role-physical, bodily pain and general health scores.

TABLE 11A
Change from Baseline in SF-36 Physical Component Score.

| Time Point | Treatment | N | LSMEAN | STDERR |
|---|---|---|---|---|
| Week 24 | Sham | 10 | 5.5 | 2.56 |
| | LYR-210 2500 μg | 16 | 7.3 | 2.39 |
| | LYR-210 7500 μg | 14 | 8.1 | 2.58 |
| Week 48 | Sham | 10 | 1.5 | 3.52 |
| | LYR-210 2500 μg | 17 | 3.8 | 2.96 |
| | LYR-210 7500 μg | 17 | 7.9 | 3.11 |

TABLE 11B
Change from Baseline in SF-36 Physical Functioning.

| Time Point | Treatment | N | LSMEAN | STDERR |
|---|---|---|---|---|
| Week 24 | Sham | 10 | 3.6 | 2.26 |
| | LYR-210 2500 μg | 16 | 6.5 | 2.10 |
| | LYR-210 7500 μg | 14 | 8.4 | 2.23 |
| Week 48 | Sham | 10 | −0.8 | 3.53 |
| | LYR-210 2500 μg | 17 | 3.3 | 2.97 |
| | LYR-210 7500 μg | 17 | 4.6 | 3.02 |

TABLE 11C
Change from Baseline in SF-36 Role Physical.

| Time Point | Treatment | N | LSMEAN | STDERR |
|---|---|---|---|---|
| Week 24 | Sham | 10 | 3.6 | 2.38 |
| | LYR-210 2500 μg | 16 | 7.3 | 2.20 |
| | LYR-210 7500 μg | 14 | 8.4 | 2.40 |
| Week 48 | Sham | 10 | 1.4 | 3.80 |
| | LYR-210 2500 μg | 17 | 4.3 | 3.18 |
| | LYR-210 7500 μg | 17 | 7.6 | 3.30 |

TABLE 11D
Change from Baseline in SF-36 Bodily Pain.

| Time Point | Treatment | N | LSMEAN | STDERR |
|---|---|---|---|---|
| Week 24 | Sham | 10 | 5.0 | 3.12 |
| | LYR-210 2500 μg | 16 | 11.2 | 2.90 |
| | LYR-210 7500 μg | 14 | 10.6 | 3.04 |
| Week 48 | Sham | 10 | 4.4 | 4.34 |
| | LYR-210 2500 μg | 17 | 5.0 | 3.61 |
| | LYR-210 7500 μg | 17 | 11.5 | 3.70 |

TABLE 11E
Change from Baseline in SF-36 General Health.

| Time Point | Treatment | N | LSMEAN | STDERR |
|---|---|---|---|---|
| Week 24 | Sham | 10 | 7.0 | 3.32 |
| | LYR-210 2500 μg | 16 | 8.4 | 3.13 |
| | LYR-210 7500 μg | 14 | 8.5 | 3.29 |
| Week 48 | Sham | 10 | 7.0 | 3.87 |
| | LYR-210 2500 μg | 17 | 6.1 | 3.33 |
| | LYR-210 7500 μg | 17 | 7.6 | 3.40 |

Conclusions: LYR-210 may improve the quality of life of surgically naïve CRS patients who failed previous medical management. Furthermore, results of SF-36v2 scores indicate a statistically significant higher quality of life during and/or after LYR-210 7500 μg treatment as compared to pretreatment scores, e.g., improvement in mental component, vitality, social functioning, emotional and overall mental health. Results of SF-36v2 scores indicate a statistically significant higher quality of life during and/or after LYR-210 2500 μg treatment as compared to pretreatment scores, e.g., improvement in mental component, social functioning, emotional and overall mental health. Results of SF-36v2 scores indicate a statistically significant higher quality of life during and/or after LYR-210 7500 μg treatment as compared to pretreatment scores, e.g., improvement in physical component, physical functioning, role physical, and bodily pain. Results of SF-36v2 scores indicate a statistically significant higher quality of life during and/or after LYR-210 2500 μg treatment as compared to pretreatment scores, e.g., bodily pain.

Thus, using preset MCID values in specific treatments as part of measuring improvements in SNOT-22 domains allows for a better assessment of how a particular treatment impacts quality-of-life of CRS patients. To illustrate this point, consider a hypothetical scenario where two competing interventions for CRS have similar total SNOT-22 improvements, e.g., surgery or LY10, where both achieves an MCID of improvement in the sleep dysfunction domain while other nonsurgical treatments do not. When providing clinical options for a CRS patient who wants significant impairment in sleep dysfunction, the clinician may be more inclined to promote treatments that will achieve an MCID in the sleep domain, even if such treatment may not improve as much in at least one other symptom domains.

To evaluate the treatment effect of LYR-210 on sinonasal mucosal inflammation, patients underwent paranasal sinus MRI at baseline and at the end of treatment (week 24). Since LYR-210 was placed in the middle meatus and is designed to locally deliver MF to the surrounding nasal tissue, the effect of LYR-210 on the inflammation in the ethmoids, the nearest sinus to the middle meatus, was examined. LYR-210 demonstrated improvement in bilateral ethmoid Zinreich MRI scores at week 24 in a dose-dependent manner, with the LYR-210 (7500 μg) demonstrating significant improvement compared to control (FIG. 7).

Time-to-first rescue treatment use was also assessed, with the need for rescue treatment at the physician's discretion. In other words, patients at risk of a sinus “event,” as determined by the inventors and described herein at least in part (see Table 2), were divided into at least 3 trial groups: no treatment, LYR-210 (2500 μg) and LYR-210 (7500 μg). Therefore, as an at risk patient has their first adverse event, they are administered rescue medication.

As another example, numbers of patients having no such health event are listed below the chart in FIG. 8.

LYR-210 showed a dose dependent reduction in the need for rescue treatment, with the LYR-210 (7500 μg) arm requiring significantly less rescue treatment than control (hazard ratio =P<0.05). Only 1 patient administered LYR-210 (7500 μg) and 2 patients administered LYR-210 (2500 μg) required a rescue treatment compared to 7 patients in the control arm over the 24-week treatment period (FIG. 8). Furthermore, the first incidence of rescue treatment in the control arm occurred at week 2, while the only patient in the LYR-210 (7500 μg) that received rescue treatment did not require it until after 18 weeks of treatment.

### DISCUSSION OF EXPERIMENTAL RESULTS

LYR-210 was safe and well-tolerated by patients throughout the entire 24-week treatment period at both doses. No treatment-related SAEs were reported and all treatment-related AEs that occurred were in line with the known safety profile of MF. 2° In this study, 80% of administered LYR-210 drug matrices were retained at week 22. This indicates that the elastomeric properties of LYR-210 allowed for prolonged contact with the sinonasal mucosa and thus, therapeutically effective local MF delivery throughout the 24-week treatment period.

A composite score focused on the cardinal symptoms of CRS and the SNOT-22 both provide information on the impact of CRS on patients and the effectiveness of treatments. In this study, LYR-210 (7500 μg) demonstrated significant improvements in nasal blockage, facial pain/pressure, and nasal discharge at weeks 16, 20, and 24 compared to saline irrigation control. Moreover, LYR-210 (7500 μg) improved smell in patients who exhibited moderate-to-severe anosmia at baseline (>2 baseline score in loss of smell). As only a subset of enrolled patients in this study had impaired sense of smell, the 3CS composite score of nasal blockage, facial pain/pressure, and nasal discharge may be a more appropriate endpoint for measuring symptom improvement for surgically naïve CRS patients with and without nasal polyps, as they are more reliably present in this study population. LYR-210 (7500 μg) demonstrated symptom improvement in the 3CS composite score that was statistically significant compared to saline irrigation control at weeks 12, 16, 20, and 24. LYR-210 (7500 μg) also achieved statistically significant improvement in SNOT-22 at weeks 8, 16, 20, and 24 compared to saline irrigation control. LYR-210 (7500 μg) significantly reduced ethmoid sinus opacification and the need for rescue treatment compared to control.

Moreover, patients administered bilateral LYR-210 (7500 μg) exhibited reduced mucosal inflammation as assessed by MRI. Compared to control, LYR-210 (7500 μg) significantly reduced the need for and time to first rescue treatment with only one patient in the LYR-210 (7500 μg) arm requiring rescue treatment after 18 weeks.

Despite curtailed enrollment at 67 patients, LYR-210 demonstrated rapid and durable dose-dependent symptom improvement throughout 24 weeks. The primary efficacy endpoint of the LANTERN phase 2 study is the mean CFBL in the 4CS score at week 4, which was −2.2 points for LYR-210 (2500 μg) and −2.5 points for LYR-210 (7500 μg) (FIG. 5A). The rationale for selecting week 4 as the primary efficacy endpoint was to be in accordance with the regulatory precedent at the time of the LANTERN study design, which was 4 weeks for FDA-approved steroid-eluting sinonasal implants.^{21, 22} While neither dose achieved statistical significance at week 4 compared to saline irrigation control, a clinically meaningful treatment effect was observed for both doses of LYR-210 at week 4, demonstrated by an improvement greater than half of the standard deviation of the baseline value (−0.8 point in CFBL in 4CS score), a statistical rule that is widely accepted for QOL assessments [Norman et a12003; Soler et a12010; Philips et a12018; Levi et al 2017]. LYR-210 (7500 μg) demonstrated significant improvement in 4CS score at weeks 16, 20, and 24 compared to saline irrigation control, indicating a long-acting and durable treatment effect. In the SNOT-22 assessment, 70% of patients administered LYR-210 (7500 μg) achieved the MCID in SNOT-22 at week 4, which persisted and grew more pronounced with 100% of patients achieving MCID by week 24 (Table 3A). A larger multicenter, randomized, blinded, controlled clinical study is planned to further evaluate the efficacy of LYR-210 (7500 μg) in CRS patients.

The control arm in this study reported symptom improvement based on the 4CS and SNOT-22 assessments, particularly within the first 4 weeks. This response can be attributed to the procedure, wherein Patients received decongestant and had their sinuses cleaned, as well as daily patient-administered nasal saline irrigations. Nasal saline irrigation is a mainstay guideline-driven standard of care therapy in CRS^{2, 3} and has been shown to improve SNOT-22 scores by approximately 20 points from baseline when used as a monotherapy.²³ Importantly, Patients in this study demonstrated high compliance (mean 82.6%±26.7%, median 94.6%) with the nasal saline irrigation regimen throughout the 24-week treatment period, which is much higher than real world use.⁸

The LANTERN Phase 2 study is the first clinical trial to show a dose response with an implantable sinonasal treatment in CRS patients that provides sustained corticosteroid therapy over 24 weeks from a single administration. LYR-210 demonstrated a reproducible treatment effect in surgically naïve CRS patients regardless of polyp status in 2 different clinical studies. The CFBL in SNOT-22 over the 24-week treatment period observed for LYR-210 (2500 μg) in the LANTERN Phase 2 study is consistent with that of LYR-210 of the same dose in a Phase 1 study¹³, further validating the findings in this clinical study and the XTreo™ drug delivery platform, whereby sustained delivery of medication for many months can be achieved.

Current FDA-approved steroid-eluting sinonasal implants provide up to 90 days of treatment to the sinuses of CRSwNP patients who have had FESS^{21, 22}, neglecting the vast majority of patients suffering with CRS, those who are pre-surgical and without nasal polyps. Consistent high dose drug delivery to the inflamed sinonasal mucosa over an extended period with patient-independent actuation has been identified as a factor for achieving adequate symptom control in CRS.^{24, 25} LYR-210 (7500 μg) improves upon current FDA-approved steroid-eluting sinonasal implants by having an approximately 5.5-fold higher total steroid dose, and demonstrating up to 24 weeks of clinical benefit in a single administration in CRS patients independent of polyp status. Moreover, LYR-210 placement in the middle meatus enables long-acting therapy directly to the site of CRS inflammation, which may address limitations of INCS. As LYR-210 is positioned early in the CRS treatment paradigm, it may provide a promising new therapy for optimizing medical management to control CRS (i.e. patient's symptoms).

### CONCLUSIONS AND HIGHLIGHTS

LYR-210 is the first anti-inflammatory implantable drug treatment to demonstrate up to 24 weeks of benefit in surgically naïve CRS patients independent of polyp status. LYR-210 is the first implantable sinonasal treatment to achieve up to 24 weeks of benefit from a single administration in surgically naïve CRS patients with and without nasal polyps. LYR-210 may be a promising therapeutic option for patients who have failed medical management as an alternative to invasive sinus surgery or systemic treatments. The treatment effect can continue even after the implants are removed. Importantly, removal was not associated with any immediate increase in adverse symptoms.

Chronic rhinosinusitis (CRS) is described in the literature as an “unrecognized epidemic” due to its high prevalence, its substantial impact on patient quality of life, and significant limitations of treatment options.¹ CRS is characterized by sinonasal inflammation with patients exhibiting at least 2 of the 4 cardinal symptoms (nasal blockage, facial pain/pressure, nasal discharge, and loss of smell) for longer than 12 weeks.^{2, 3} Historically, CRS has been divided into 2 phenotypes based on the presence or absence of nasal polyps (CRSwNP and CRSsNP, respectively). More recently, CRS has been divided into endotypes defined by the molecular mechanism that underwrites the pattern on inflammation in the tissue.^{4, 5} From this perspective, 85% of Western CRSwNP patients exhibit Type 2 (T2) inflammation, with significant eosinophilia.⁶ Western CRSsNP is more heterogeneous with approximately 50% of patients demonstrating T2 inflammation.⁶ Importantly, the T2 CRS endotype has been associated with severe symptoms and a high rate of treatment failure.³ As there is no cure for CRS, current medical and surgical interventions are directed toward managing symptoms of CRS by reducing inflammation and eradicating any existing infection. Moreover, there are no current FDA-approved drug therapies to treat CRSsNP, although some drugs approved for nasal polyps are used off-label in this population.

### REFERENCES

Norman et al 2003; Soler et al 2010; Philips et al 2018; Levi et al 2017.
1. Tan B K, Kern R C, Schleimer R P, Schwartz B S. Chronic rhinosinusitis: the unrecognized epidemic. Am J Respir Crit Care Med. Dec. 1 2013; 188(11):1275-7. doi:10.1164/rccm.201308-1500ED
2. Orlandi R R, Kingdom T Y, Hwang P H. International Consensus Statement on Allergy and Rhinology: Rhinosinusitis Executive Summary. Int Forum Allergy Rhinol. February 2016; 6 Suppl 1:S3-21. doi:10.1002/alr.21694
3. Fokkens W J, Lund V J, Hopkins C, et al. European Position Paper on Rhinosinusitis and Nasal Polyps 2020. Rhinology. Feb. 20 2020; 58(Suppl S29):1-464. doi:10.4193/Rhin20.600
4. Tomassen P, Vandeplas G, Van Zele T, et al. Inflammatory endotypes of chronic rhinosinusitis based on cluster analysis of biomarkers. J Allergy Clin Immunol. May 2016; 137(5):1449-1456 e4. doi:10.1016/j.jaci.2015.12.1324
5. Tan B K, Min J Y, Hulse K E. Acquired Immunity in Chronic Rhinosinusitis. Curr Allergy Asthma Rep. July 2017; 17(7):49. doi:10.1007/s11882-017-0715-0
6. Stevens W W, Peters A T, Tan B K, et al. Associations Between Inflammatory Endotypes and Clinical Presentations in Chronic Rhinosinusitis. J Allergy Clin Immunol Pract. November-December 2019; 7(8):2812-2820 e3. doi:10.1016/j.jaip.2019.05.009
7. Moeller W, Schuschnig U, Meyer G, et al. Ventilation and aerosolized drug delivery to the paranasal sinuses using pulsating airflow—a preliminary study. Rhinology. December 2009; 47(4):405-12. doi:10.4193/Rhin08.180
8. Nabi S, Rotenberg B W, Vukin I, Payton K, Bureau Y. Nasal spray adherence after sinus surgery: problems and predictors. J Otolaryngol Head Neck Surg. April 2012; 41 Suppl 1:S49-55.
9. Young L C, Stow N W, Zhou L, Douglas R G. Efficacy of medical therapy in treatment of chronic rhinosinusitis. Allergy Rhinol (Providence). 2012; 3(1):e8-e12. doi:10.2500/ar.2012.3.0027
10. Schaitkin B, May M, Shapiro A, Fucci M, Mester S J. Endoscopic sinus surgery: 4-year follow-up on the first 100 patients. Laryngoscope. October 1993; 103(10):1117-20. doi: 10.1288/00005537-199310000-00007
11. Sharma U, Concagh D, Core L, et al. The development of bioresorbable composite polymeric implants with high mechanical strength. Nat Mater. January 2018; 17(1):96-103. doi:10.1038/nmat5016
12. Stammberger H. Endoscopic endonasal surgery—concepts in treatment of recurring rhinosinusitis. Part I. Anatomic and pathophysiologic considerations. Otolaryngol Head Neck Surg. February 1986; 94(2):143-7. doi:10.1177/019459988609400202
13. Douglas R G, Psaltis A J, Rimmer J, Kuruvilla T, Cervin A, Kuang Y. Phase 1 clinical study to assess the safety of a novel drug delivery system providing long-term topical steroid therapy for chronic rhinosinusitis. Int Forum Allergy Rhinol. April 2019; 9(4):378-387. doi:10.1002/alr.22288
14. Hopkins C, Gillett S, Slack R, Lund V J, Browne J P. Psychometric validity of the 22-item Sinonasal Outcome Test. Clin Otolaryngol. October 2009; 34(5):447-54. doi:10.1111/j 1749-4486.2009.01995.x
15. Zinreich S J. Imaging for staging of rhinosinusitis. Ann Otol Rhinol Laryngol Suppl. May 2004; 193:19-23. doi:10.1177/00034894041130s506
16. Toma S, Hopkins C. Stratification of SNOT-22 scores into mild, moderate or severe and relationship with other subjective instruments. Rhinology. June 2016; 54(2):129-33. doi:10.4193/Rhin15.072
17. Mandavinia M, Schleimer R P, Keshavarzian A. Sleep disruption in chronic rhinosinusitis. Expert Rev Anti Infect Ther. May 2017; 15(5):457-465. doi: 10.1080/14787210.2017.1294063
18. DeConde A S, Mace J C, Bodner T, et al. SNOT-22 quality of life domains differentially predict treatment modality selection in chronic rhinosinusitis. Int Forum Allergy Rhinol. December 2014; 4(12):972-9. doi:10.1002/alr.21408
19. Chowdhury N I, Mace J C, Bodner T E, et al. Investigating the minimal clinically important difference for SNOT-22 symptom domains in surgically managed chronic rhinosinusitis. Int Forum Allergy Rhinol. December 2017; 7(12):1149-1155. doi:10.1002/alr.22028
20. Zitt M, Kosoglou T, Hubbell J. Mometasone furoate nasal spray: a review of safety and systemic effects. Drug Saf. 2007; 30(4):317-26. doi:10.2165/00002018-200730040-00004
21. Marple B F, Smith T L, Han J K, et al. Advance II: a prospective, randomized study assessing safety and efficacy of bioabsorbable steroid-releasing sinus implants. Otolaryngol Head Neck Surg. June 2012; 146(6):1004-11. doi:10.1177/0194599811435968
22. Kern R C, Stolovitzky J P, Silvers S L, et al. A phase 3 trial of mometasone furoate sinus implants for chronic sinusitis with recurrent nasal polyps. Int Forum Allergy Rhinol. April 2018; 8(4):471-481. doi:10.1002/alr.22084
23. Behera S K, Radhakrishnan S T, Swain S. Nasal irrigation using saline at room temperature or body temperature: which is more beneficial in chronic rhinosinusitis? Nasal irrigation, Temperature, Chronic rhinosinusitis, Saccharine transit time. 2019. 2019 Jun. 27 2019; 5(4):4. doi:10.18203/issn.2454-5929.ij0hns20192720
24. Liang J, Lane A P. Topical Drug Delivery for Chronic Rhinosinusitis. Curr Otorhinolaryngol Rep. Mar. 1 2013; 1(1):51-60. doi:10.1007/s40136-012-0003-4
25. Illum L. Nasal drug delivery: new developments and strategies. Drug Discov Today. Dec. 1 2002; 7(23):1184-9. doi:10.1016/s1359-6446(02)02529-1

### LYR-210 Pharmacokinetics and Drug Release

Plasma MF concentrations were evaluated from patients administered bilateral LYR-210 (2500 μg) or LYR-210 (7500 μg) at 1-hour post-placement (day 1), and days 2, 3, 7, 14, 21, 28, 42, and 56. FIG. 10 shows constant and dose-dependent MF plasma concentrations throughout the 56-day study duration. For the LYR-210 (2500 μg) and LYR-210 (7500 μg) groups, the plasma MF concentrations peaked at Day 2 (Cₘₐₓ=21.5 μg/mL) and Day 3 (Cₘₐₓ=58.9 μg/mL), respectively. The plasma MF concentrations dropped slightly and plateaued after Day 7. For this 56-day study, LYR-210 delivered a consistent daily dose of MF, achieving steady-state concentrations (Cₛₛ) of 12.2 μg/mL and 41.2 μg/mL for LYR-210 (2500 μg) and LYR-210 (7500 μg), respectively.

The percent total MF remaining on LYR-210 matrices removed from patients at day 56 was determined by HPLC-UV. Approximately 80% of the total MF loaded onto LYR-210 matrices remained after 56 days of treatment, revealing 18.3±5.2% and 19.1±4.7% of the total MF dose was released from LYR-210 (2500 μg) and LYR-210 (7500 μg) matrices, respectively.

TABLE 12
Arithmetic Mean Plasma MF Concentrations (pg/mL).

| | | Week 4 | Week 12 |
|---|---|---|---|
| LYR-210 (2500 μg) | n | 20 | 16 |
| | Mean | 13.6 | 21.7 |
| | CV % | 38 | 36 |
| LYR-210 (7500 μg) | n | 16 | 14 |
| | Mean | 45.4 | 30.9 |
| | CV % | 25 | 39 |

Table 12 demonstrates the ability of the implants comprising 7500 micrograms of mometasone furoate (MF) to deliver drug out to 12 weeks.

### ADDITIONAL BENEFITS OF TREATMENTS

Additional benefits of treatments comprising Mometasone furoate 7500 μg dosages per matrix (i.e. 7500 μg each side of nose for a total of 15000 μg per patient, are described below.

A dose response is observed in patients' having two Mometasone furoate implants, i.e. 7500 μg each, which results in greater improvement than patients' having two 2500 μg implants, e.g., 7500 μg improves chronic rhinosinusitis (CRS) symptoms in patients having a moderate/severe condition as measured by one or more categories of CRS symptoms at week 24. Improved CRS symptom categories specifically include, but are not limited to: 3CS, 4CS, nasal blockage, nasal discharge, facial pain, and SNOT-22 scores.

LYR-210 (7500 μg) treatment does not worsen patient's CRS symptoms from baseline to a more severe category in 3CS, 4CS, nasal blockage, nasal discharge, facial pain, or SNOT-22 at week 24.

LYR-210 (7500 μg) may result in mild or no CRS symptoms at week 24, Based on 3CS. LYR-210 (7500 μg) shows improvement in some patients' SNOT-22 scores by 50% from baseline at week 24.

LYR-210 (7500 μg) converts surgical candidates to non-surgical candidates by the end of treatment (week 24). Conversion is determined, at least in part, when a change to non-surgical candidacy is defined by 3CS≤4 at week 24.

In some preferred embodiments, a treated surgical candidate patient is no longer a surgical candidate when the patient's 3CS≤4 at week 24.

LYR-210 (7500 μg) may reduce incidence of acute exacerbations of chronic sinusitis during the treatment period.

Mometasone furoate released from LYR-210 (7500 μg) is detectable in plasma out to at least week 24.

### ADDITIONAL DATA ANALYSIS

The following data supports several benefits of patient treatments comprising 7500 μg

Mometasone furoate implants, including but not limited to comparisons to patients treated with implants comprising 2500 μg Mometasone furoate implants.

LANTERN: LYR-210 (7500 μg) Improves CRS Symptom Severity at Week 24 (Based on 3CS score) in Patients Having Moderate/Severe Disease at Baseline.

Data analysis showed improvement in at least one 3CS symptom category in some patients having severe disease at baseline improved to moderate or improved from having moderate disease to mild disease at week 24. Data analysis showed improvement in at least two 3CS symptom categories in some patients having severe disease to mild disease or moderate disease to no/none disease at week 24. See, Table 13 below.

Zero patients treated with LYR-210 (7500 μg) worsened to a more severe category at week 24 (based on 3CS score).

In particular, patients treated with 7500 μg Mometasone furoate implants that had severe disease at baseline improved in at least one 3CS severity category by week 24. More specifically, 5/7 patients (71.4%) severe disease improved to moderate disease; 1 patient (14.3%) having severe disease improved to having mild disease; and 1 patient (14.3%) having Severe disease improved to having no disease symptoms. See, Table 13 below.

A dose response was observed for certain disease categories and timepoints. In particular, a dose-response was observed with patients that had severe disease at baseline such that LYR-210 (2500 μg) treatment was not as effective as LYR-210 (7500 μg), at week 24, wherein 21.4% of the patients having severe disease continued to have severe symptoms. In comparison, 4/12 (33.3%) sham/control patients that had severe disease at baseline continued to have severe disease at week 24.

Further, unlike the 2500 μg treated patients, 12/13 (92.3%) of LYR-210 (7500 μg)-treated patients that had moderate disease at baseline, showed improvement to mild or no (none) disease at week 24. In comparison, 8/11 (72.7%) sham/control patients that had moderate disease at baseline experienced either no improvement (i.e., remained moderate) or worsened (to severe) at week 24. See, Table 13 below.

TABLE 13
LANTERN: Shift in 3 CS from Baseline to Week
24 (Last Observation Carried Forward (LOCF)).

| | | | Week 24 | | | | | | | |
|---|---|---|---|---|---|---|---|---|---|---|
| | | | None | | Mild | | Moderate | | Severe | |
| Baseline | Treatment | N | n | % | n | % | n | % | n | % |
| Mild | LYR-210 7500 μg | 1 | 0 | 0.0% | 1 | 100.0% | 0 | 0.0% | 0 | 0.0% |
| | LYR-210 2500 μg | 2 | 0 | 0.0% | 2 | 100.0% | 0 | 0.0% | 0 | 0.0% |
| Moderate | LYR-210 7500 μg | 13 | 1 | 7.7% | 11 | 84.6% | 1 | 7.7% | 0 | 0.0% |
| | LYR-210 2500 μg | 7 | 1 | 14.3% | 4 | 57.1% | 2 | 28.6% | 0 | 0.0% |
| | Sham | 11 | 0 | 0.0% | 3 | 27.3% | 7 | 63.6% | 1 | 9.1% |
| Severe | LYR-210 7500 μg | 7 | 1 | 14.3% | 1 | 14.3% | 5 | 71.4% | 0 | 0.0% |
| | LYR-210 2500 μg | 14 | 1 | 7.1% | 2 | 14.3% | 8 | 57.1% | 3 | 21.4% |
| | Sham | 12 | 0 | 0.0% | 3 | 25.0% | 5 | 41.7% | 4 | 33.3% |

None = [0, 1.5);
Mild = [1.5, 4.5);
Moderate = [4.5, 7.5);
Severe = [7.5, 9]

### LANTERN: LYR-210 (7500 μg) Improves Nasal Blockage Severity at Week 24.

Data analysis showed improvement of nasal blockage severity at week 24 in some patients having severe disease at baseline improved to having moderate disease or patients having moderate disease at baseline improved to having mild disease at week 24. Data analysis showed improvement in at least in two symptom categories in some patients having severe disease that improved to mild disease at week 24. Data analysis showed improvement in at least in three symptom categories in some patients having severe disease that improved to no/none disease at week 24.

No patient treated with LYR-210 (7500 μg) worsened to a more severe category at week 24.

More specifically, 100% of patients treated with LYR-210 7500 μg Mometasone furoate implants that had severe disease at baseline improved in at least one severity category by week 24. In particular, 7/12 (58.3%) patients having severe disease improved to moderate disease by week 24 while 4/12 (33.3%) of patients having severe disease improved to mild disease by week 24. Even further, 1/12 (8.3%) of patients having severe disease improved to having no/none disease by week 24. In comparison, 4/14 (28.6%) sham/control patients that had severe disease at baseline remained with severe disease at week 24. See, Table 14 below.

TABLE 14
LANTERN: Shift in Nasal Blockage from Baseline to Week 24 (LOCF).

| | | | Week 24 | | | | | | | |
|---|---|---|---|---|---|---|---|---|---|---|
| | | | None | | Mild | | Moderate | | Severe | |
| Baseline | Treatment | N | n | % | n | % | n | % | n | % |
| Moderate | LYR-210 7500 μg | 9 | 0 | 0.0% | 7 | 77.8% | 2 | 22.2% | 0 | 0.0% |
| | LYR-210 2500 μg | 9 | 0 | 0.0% | 4 | 44.4% | 4 | 44.4% | 1 | 11.1% |
| | Sham | 9 | 0 | 0.0% | 2 | 22.2% | 6 | 66.7% | 1 | 11.1% |
| Severe | LYR-210 7500 μg | 12 | 1 | 8.3% | 4 | 33.3% | 7 | 58.3% | 0 | 0.0% |
| | LYR-210 2500 μg | 14 | 1 | 7.1% | 4 | 28.6% | 4 | 28.6% | 5 | 35.7% |
| | Sham | 14 | 0 | 10.0% | 4 | 28.6% | 6 | 42.9% | 4 | 28.6% |

None = [0, 0.5);
Mild = [0.5, 1.5);
Moderate = [1.5, 2.5);
Severe = [2.5, 3]

### LANTERN: LYR-210 (7500 μg) Improves Nasal Discharge Severity at Week 24.

Data analysis showed improvement in at least one symptom category in some patients having severe disease at baseline to having moderate disease or patients having moderate disease at baseline improving to having mild disease at week 24. Data analysis showed improvement in at least in two symptom categories, in some patients having severe disease at baseline to having mild disease at week 24. See Table 15.

No patient treated with LYR-210 (7500 μg) worsened to a more severe category of nasal discharge at week 24.

More specifically, 100% of patients treated with 7500 μg Mometasone furoate implants that had severe disease at baseline improved in at least one severity category by week 24. In particular, ⅝ (62.5%) patients with severe disease improved to having moderate disease while ⅜ (37.5%) patients with severe disease improved to having mild disease. 11/12 (91.6%) LYR-210 (7500 μg)-treated patients that were moderate at baseline improved to having mild or no/none disease at week 24. In comparison, 4/13 (30.8%) sham/control patients that had severe disease at baseline continued to have severe disease at week 24. See Table 15.

TABLE 15
LANTERN: Shift in Nasal Discharge from Baseline to Week 24 (LOCF).

| | | | Week 24 | | | | | | | |
|---|---|---|---|---|---|---|---|---|---|---|
| | | | None | | Mild | | Moderate | | Severe | |
| Baseline | Treatment | N | n | % | n | % | n | % | n | % |
| Mild | LYR-210 7500 μg | — | 1 | 100.0% | 0 | 0.0% | 0 | 0.0% | 0 | 0.0% |
| | LYR-210 2500 μg | 2 | 0 | 0.0% | 1 | 50.0% | 1 | 50.0% | 0 | 0.0% |
| Moderate | LYR-210 7500 μg | 12 | 1 | 8.3% | 10 | 83.3% | 1 | 8.3% | 0 | 0.0% |
| | LYR-210 2500 μg | 6 | 1 | 16.7% | 3 | 50.0% | 2 | 33.3% | 0 | 0.0% |
| | Sham | 10 | 0 | 0.0% | 5 | 50.0% | 4 | 40.0% | 1 | 10.0% |
| Severe | LYR-210 7500 μg | 8 | 0 | 0.0% | 3 | 37.5% | 5 | 62.5% | 0 | 0.0% |
| | LYR-210 2500 μg | 15 | 0 | 0.0% | 5 | 133.3% | 7 | 46.7% | 3 | 20.0% |
| | Sham | 13 | 0 | 0.0% | 3 | 23.1% | 6 | 46.2% | 4 | 30.8% |

None = [0, 0.5);
Mild = [0.5, 1.5);
Moderate = [1.5, 2.5);
Severe = [2.5, 3]

LANTERN: Shift in Improvement of Facial Pain from Baseline to Week 24.

LYR-210 (7500 μg) reduces facial pain severity in patients at week 24. At week 24, data analysis showed improvement in at least one symptom category in some patients having severe disease at baseline who improved to having moderate disease or patients having moderate disease at baseline improving to patients having mild disease at week 24. Data analysis showed improvement in at least two symptom categories in some patients having moderate disease at baseline who improved to having no/none disease at week 24. Data analysis showed improvement in at least three symptom categories in some patients having severe disease at baseline who improved to having no/none disease at week 24. See, Table 16, below.

No patient treated with LYR-210 (7500 μg) worsened to a more severe category in facial pain at week 24.

More specifically, 100% of patients treated with 7500 μg Mometasone furoate implants that had severe disease at baseline improved in at least one severity category by week 24. In particular, ¾ (75%) patients with severe disease improved to having moderate disease while ¼ (25%) patients with severe disease improved to having no/none disease. Further, 12/15 (80%) LYR-210 (7500 μg)-treated patients that had moderate disease at baseline improved to having mild or no/none disease at week 24. In comparison to 3/12 (25%) sham/control patients that had severe disease at baseline remained with severe disease at week 24. See, Table 16, below.

TABLE 16
LANTERN: Shift in improvement of Facial Pain from Baseline to Week 24 (LOCF).

| | | | Week 24 | | | | | | | |
|---|---|---|---|---|---|---|---|---|---|---|
| | | | None | | Mild | | Moderate | | Severe | |
| Baseline | Treatment | N | n | % | n | % | n | % | n | % |
| None | LYR-210 7500 μg | 2 | 21 | 100.0% | 0 | 0.0% | 0 | 0.0% | 0 | 0.0% |
| None | LYR-210 2500 μg | 2 | 21 | 100.0% | 0 | 0.0% | 0 | 0.0% | 0 | 0.0% |
| None | Sham | 1 | 1 | 100.0% | 0 | 0.0% | 0 | 0.0% | 0 | 0.0% |
| Mild | LYR-210 2500 μg | 2 | 1 | 50.0% | 1 | 50.0% | 0 | 0.0% | 0 | 0.0% |
| Mild | Sham | 3 | 0 | 0.0% | 2 | 66.7% | 1 | 33.3% | 0 | 0.0% |
| Moderate | LYR-210 7500 μg | 15 | 2 | 13.3% | 10 | 66.7% | 3 | 20.0% | 0 | 0.0% |
| Moderate | LYR-210 2500 μg | 7 | 2 | 28.6% | 2 | 28.6% | 2 | 28.6% | 1 | 14.3% |
| Moderate | Sham | 7 | 0 | 0.0% | 3 | 42.9% | 3 | 42.9% | 1 | 14.3% |
| Severe | LYR-210 7500 μg | 4 | 1 | 25.0% | 0 | 0.0% | 3 | 75.0% | 0 | 0.0% |
| Severe | LYR-210 2500 μg | 12 | 2 | 16.7% | 2 | 16.7% | 5 | 41.7% | 3 | 25.0% |
| Severe | Sham | 12 | 0 | 0.0% | 3 | 25.0% | 6 | 50.0% | 3 | 25.0% |

None = [0, 0.5);
Mild = [0.5, 1.5);
Moderate = [1.5, 2.5);
Severe = [2.5, 3]

LANTERN: Patients Treated with Two 7500 μg Implants Showed an Improvement in 3CS Symptom Responses at Week 24.

One hundred percent (100%) of patients treated with two LYR-210 7500 μg Mometasone furoate implants reported at least a 1-point reduction in 3CS severity symptoms at week 24. This improvement represents a statistically significant improvement over the 65.2% improvement in control/sham patients.

Further, 90.5% of LYR-210 (7500 μg)-treated patients reported at least a 2-point reduction in 3CS severity symptoms at week 24. This improvement is statistically significant compared to a 43.5% improvement in control patients.

Moreover, a dose response was demonstrated where patients having 7500 μg implants showed a greater response that patients having 2500 μg implants. See Table 17, below.

TABLE 17
LANTERN: 3CS Response at Week 24 (LOCF).

| RSP_DEF | TRT01P | N | RSP | PCT | XPL_FISH |
|---|---|---|---|---|---|
| >=1-point | LYR-210 7500 μg | 21 | 21 | 100.0% | 0.0028 |
| Reduction | LYR-210 2500 μg | 23 | 17 | 73.9% | 0.3747 |
| | Sham | 23 | 15 | 65.2% | . |
| >=2-point | LYR-210 7500 μg | 21 | 19 | 90.5% | 0.0011 |
| Reduction | LYR-210 2500 μg | 23 | 15 | 65.2% | 0.1181 |
| | Sham | 23 | 10 | 43.5% | . |

### LANTERN: Patients Having Two 7500 μg Implants Demonstrated a 4CS Dose Response in Improvement Compared Patients Having Two 2500 μg Implants Observed at Week 24.

One hundred percent (100%) of patients treated with two LYR-210 7500 μg Mometasone furoate implants reported at least a 1-point reduction in 4CS severity symptoms at week 24. This improvement is a statistically significant improvement over the 65.2% improvement in control patients. There was a dose response demonstrated of 7500 μg-treated patients over 2500 μg-treated patients.

Further, 90.5% of LYR-210 (7500 μg)-treated patients reported at least a 2-point reduction in 4CS severity symptoms at week 24. This improvement is statistically significant compared to 52.2% improvement in control patients.

Even further, 76.2% of the LYR-210 (7500 μg)-treated patients reported at least a 3-point reduction in 3CS at week 24. This improvement is statistically significant compared to 43.5% improvement in control patients. See Table 18, below.

TABLE 18
LANTERN: 4CS Response at Week 24 (LOCF).

| RSP_DEF | TRT01P | N | RSP | PCT | XPL_FISH |
|---|---|---|---|---|---|
| >=1-point | LYR-210 7500 μg | 21 | 21 | 100.0% | 0.0028 |
| Reduction | LYR-210 2500 μg | 23 | 17 | 73.9% | 0.3747 |
| | Sham | 23 | 15 | 65.2% | . |
| >=2-point | LYR-210 7500 μg | 21 | 19 | 90.5% | 0.0060 |
| Reduction | LYR-210 2500 μg | 23 | 16 | 69.6% | 0.1826 |
| | Sham | 23 | 12 | 52.2% | . |
| >=3-point | LYR-210 7500 μg | 21 | 16 | 76.2% | 0.0280 |
| Reduction | LYR-210 2500 μg | 23 | 12 | 52.2% | 0.3842 |
| | Sham | 23 | 10 | 43.5% | . |

### LANTERN: SNOT-22 MCID Response (Improvement≥8.9) at Week 24.

100% of patients treated with 7500 μg Mometasone furoate implants improved by at least 8.9 points in SNOT-22 scores, the minimal clinically important point difference, at week 24. This result is statistically significant compared to improvement in a sham (control) group of patients. SNOT-22 improvement is statistically significant to control (p=0.0034). See, Table 19, below.

TABLE 19
LANTERN: SNOT-22 Response (Improvement ≥8.9) at Week
24 (LOCF).

| TRT01P | N | RSP | PCT | XPL_FISH |
|---|---|---|---|---|
| LYR-210 7500 μg | 20 | 20 | 100.0% | 0.0034 |
| LYR-210 2500 μg | 23 | 16 | 69.6% | 0.5000 |
| Sham | 23 | 15 | 65.2% | . |

### LANTERN: Conversion of a Patient as a Surgical Candidate to a Nonsurgical Candidate at Week 24.

Surprisingly, at this week 24 observation timepoint patients implanted with two LYR-210 at 7500 ug, each implant, 71.4% of surgical candidates, were re-classified (changed) to nonsurgical candidates when their symptoms changed to having values of 3CS≤4. In comparison, 26.1% of control patients were re-classified based upon the same criteria. Although having an Ethmoid Zinreich Score=0 was determined to be a criterion for re-classification, none of the patients evaluated here had an Ethmoid Zinreich score =0. See, Table 20, below.

Moreover, a dose-response was observed in re-classification between patients having high dose (7500 ug) vs. low dose (2500 ug) implants.

TABLE 20
LANTERN: Conversion to Nonsurgical Candidacy at Week 24 (LOCF).
3CS ≤ 4 or Ethmoid Zinreich Score = 0.

| TRT01P | N | NONSURG | PCT | XPL_FISH |
|---|---|---|---|---|
| LYR-210 7500 μg | 21 | 15 | 71.4% | 0.0031 |
| LYR-210 2500 μg | 23 | 9 | 39.1% | 0.2651 |
| Sham | 23 | 6 | 26.1% | . |

LANTERN: Acute Exacerbation of CS during Treatment Period.

Fewer acute exacerbations of chronic sinusitis occurred in the LYR-210 (7500 μg) treatment group during a 24-week treatment period. See, Table 21, below.

TABLE 21
LANTERN: Acute Exacerbation of CS during Treatment Period.

| TRT01P | N | ACU_EXAC | PCT | XPR_FISH |
|---|---|---|---|---|
| LYR-210 7500 μg | 21 | 2 | 9.5% | 0.0883 |
| LYR-210 2500 μg | 23 | 6 | 26.1% | 0.5000 |
| Sham | 23 | 7 | 30.4% | . |

### LANTERN: Correlation Between a Change in 3CS and in SNOT-22 at Week 24.

At this week 24 observation timepoint, SNOT-22 and 3CS are strongly correlated to each other and indicate statistically significant improvement from baseline disease.

Further, 3CS provides a clinically relevant assessment of the impact of a treatment on CRS symptoms at Week 24. See, Table 22, below.

TABLE 22
LANTERN: Correlation between Change in 3CS and in SNOT-22
at Week 24

| | |
|---|---|
| CHG3CS | CHGSNT |

Pearson Correlation Coefficients, N = 40
Prob > |r| under H0: Rho = 0

| | | |
|---|---|---|
| CHG3CS | 1.00000 | 0.75530 |
| | | <.0001 |
| CHGSNT | 0.75530 | 1.00000 |
| Change from Baseline | <.0001 | |

Spearman Correlation Coefficients, N = 40
Prob > |r| under H0: Rho = 0

| | | |
|---|---|---|
| CHG3CS | 1.00000 | 0.72164 |
| | | <.0001 |
| CHGSNT | 0.72164 | 1.00000 |
| Change from Baseline | <.0001 | |

### LANTERN: At Least One Positive Response to MF Implants at Week 24.

A patient group implanted with two LYR-210 (7500 μg) implants each, demonstrated a significantly higher proportion of responders than in untreated controls (i.e. no implants or sham implants without MF) at week 24. This observation is based on the parameters listed in Table 23.

In other words, a patient was considered a responder if said patient experienced at least 1 positive response and no negative responses out of the parameters listed in Table 23.

TABLE 23
LANTERN: Any Response At Week 24 (LOCF), At Least 1 Positive
and No Negative.

| TRT01P | N | RSP | PCT | XPL_FISH |
|---|---|---|---|---|
| LYR-210 7500 μg | 21 | 15 | 71.4% | 0.0318 |
| LYR-210 2500 μg | 23 | 8 | 34.8% | 0.7290 |
| Sham | 23 | 9 | 39.1% | . |

| Parameter | Positive | Negative |
|---|---|---|
| 3CS | Reduction ≥3 | Increase ≥1 |
| Nasal blockage | Reduction ≥1 | Increase ≥0.5 |
| Nasal discharge | Reduction ≥1 | Increase ≥0.5 |
| Facial pain | Reduction ≥1 | Increase ≥0.5 |
| Loss of smell | Reduction ≥1 | Increase ≥0.5 |
| SNOT-22 | Reduction ≥8.9 and | Score >50 |
| | Score ≤20 | |
| Ethmoid Zinreich Score | Reduction ≥1 | Any increase |

LANTERN: LYR-210 (7500 μg) Treatment Results in Mild or No CRS Symptoms at Week 24 (based on 3CS).

70% of patients treated with LYR-210 (7500 μg) demonstrated improvement to mild or no (none) CRS symptoms at week 24. This is statistically significant compared to sham/control. Further, a dose-dependent effect was observed between LYR-210 2500 μg patients and LYR-210 7500 μg treated patients in this analysis.

Patients having mild disease were removed from this analysis, which amounted to removing one patient from the LYR-210 (7500 μg) group and 0 patients from the sham group. See, Table 24, below.

TABLE 24
LANTERN: 3CS Severity of None/Mild at Week 24 (LOCF).

| TRT01P | N | RSP | PCT | XPL_FISH |
|---|---|---|---|---|
| LYR-210 7500 μg | 20 | 14 | 70.0% | 0.0047 |
| LYR-210 2500 μg | 21 | 8 | 38.1% | 0.2980 |
| Sham | 23 | 6 | 26.1% | . |

None = [0, 1.5); Mild = [1.5, 4.5); Moderate = [4.5, 7.5); Severe = [7.5, 9]
XPL_FISH: 1-sided p-value obtained from a Fishers exact test.

### LANTERN: LYR-210 (7500 μg) Improves CRS Symptoms By At Least 1 Severity Category Based On 3CS.

A dose-dependent effect was observed in disease severity improvement in at least one 3CS disease category in patients at week 24 after implantation of implants.

More specifically, 90.5% of patients treated with LYR-210 (7500 μg) improved by at least one 3CS severity category at week 24. This is statistically significant compared to sham/control. See, Table 25, below.

TABLE 25
LANTERN: 3CS Severity Improvement of ≥1 Category at Week
24 (LOCF).

| TRT01P | N | RSP | PCT | XPL_FISH |
|---|---|---|---|---|
| LYR-210 7500 μg | 21 | 19 | 90.5% | 0.0027 |
| LYR-210 2500 μg | 23 | 16 | 69.6% | 0.1154 |
| Sham | 23 | 11 | 47.8% | . |

None = [0, 1.5); Mild = [1.5, 4.5); Moderate = [4.5, 7.5); Severe = [7.5, 9]
XPL_FISH: 1-sided p-value obtained from a Fishers exact test

### LANTERN: 3CS Severity of None/Mild and Improvement of ≥1 Category at Week 24.

A dose-dependent effect was observed in disease severity improvement in at least one 3CS disease category in patients who showed no (none) or mild symptoms at week 24 after implantation of implants.

More specifically, 66.7% of patients treated with LYR-210 (7500 μg) had none or mild CRS symptoms at week 24 and improved in at least one severity category in 3CS at week 24. This result is statistically significant compared to sham/control. See, Table 26, below.

TABLE 26
LANTERN: 3CS Severity of None/Mild and Improvement of
≥1 Category at Week 24. (LOCF).

| TRT01P | N | RSP | PCT | XPL_FISH |
|---|---|---|---|---|
| LYR-210 7500 μg | 21 | 14 | 66.7% | 0.0078 |
| LYR-210 2500 μg | 23 | 8 | 34.8% | 0.3747 |
| Sham | 23 | 6 | 26.1% | . |

None = [0, 1.5); Mild = [1.5, 4.5); Moderate = [4.5, 7.5); Severe = [7.5, 9]
XPL_FISH: 1-sided p-value obtained from a Fishers exact test.

### MATERIALS AND METHODS

### Investigational Products

LYR-210 is an investigational product designed and manufactured by Lyra Therapeutics, Inc. (Watertown, MA, USA). LYR-210 has a tubular mesh configuration with a repeat diamond pattern throughout that is composed of biocompatible and bioabsorbable polymers formulated to precisely control the release of up to 2500 μg (LYR-210 (2500 μg)) or 7500 μg (LYR-210 (7500 μg)) of MF gradually over 24 weeks, and gradually soften over time. The engineered elastomeric properties allow LYR-210 to dynamically expand to target anatomy, promoting continuous apposition to the surrounding mucosa for effective and consistent local MF delivery over the 24-week period. LYR-210 is placed bilaterally within the middle meatus of CRS patients, that is not distorted by prior surgical intervention, with a single-use applicator in an office-based procedure under endoscopic visualization after topical anesthesia. FIG. 9C shows LYR-210 self-expanding from a constrained state upon deployment from the applicator. LYR-210 is intended to be removed at 24 weeks, or earlier at the physician's discretion, using standard instruments.

### Study Design

CRS patients who failed previous medical management were enrolled in this multicenter, randomized, blinded, controlled, dose-ranging LANTERN Phase 2 trial to evaluate the efficacy, safety, and tolerability of LYR-210 (2500 μg) and LYR-210 (7500 μg). Patients were recruited and enrolled by 14 otolaryngology practices in Poland, Czech Republic, Australia, and New Zealand. The study was conducted in accordance with the Declaration of Helsinki and Good Clinical Practice guidelines. The study protocol and patient informed consent were reviewed and approved by the Ethics Committee of each study center in accordance with the regulatory requirements of each country. Patients signed informed consent before participating in the study.

Inclusion criteria included patients aged 18 years or older with at least 2 of the 4 cardinal symptoms (4CS) of CRS for a minimum of 12 weeks^{2, 3} and a baseline average 4CS score over the preceding 7 days of 7 or higher on a 0-12 scale. Patients exhibited purulence, inflammation, and/or nasal polyps on nasal endoscopy and had radiological evidence of sinusitis on paranasal CT or MRI. Enrolled patients had at least 2 previous trials of medical treatment for CRS, independent of the LANTERN study, including at least one course of intranasal corticosteroid sprays (INCS) for a minimum of 4 weeks; a duration based on the ICAR-2016 guidance for the “Length of appropriate medical therapy prior to ESS” (Orlandi R R, Kingdom T T, Hwang P H. International Consensus Statement on Allergy and Rhinology: Rhinosinusitis Executive Summary. Int Forum Allergy Rhinol. February 2016; 6 Suppl 1:S3-21.) Enrolled patients in this trial reported to have used an average of 1.9 courses of INCS (median: 2, maximum: 5) prior to screening in the past year alone. LYR-210 (7500 μg) achieved clinically significant improvements up to 24 weeks without INCS compared to control, supporting its potential use as a monotherapy. Thus, a potential advantage for LYR-210 is the opportunity to eliminate patient compliance issues with INCS in the real world and lessen treatment burden. 100% of patients administered LYR-210 (7500 μg) achieved the MCID in SNOT-22 at week 24, which was superior to either the LYR-210 (2500 μg) and control arms. While the data reported in this study is analyzed using 8.9 points as the MCID for SNOT-22¹⁴, the MCID has also been reported to be 12 points in medically managed CRS patients²⁶. Using this alternative MCID of 12 points, 90% of patients administered LYR-210 (7500 μg) achieved this MCID at week 24 compared to 65% of patients in the control arm in this study.

Exclusion criteria for patients included history of prior FESS, nasal endoscopic evidence of significant mucosal injury (e.g. ulceration or erosion), nasal septal perforation, severe nasal blockage by nasal polyps that prevented access to or visualization of the middle meatus, concurrent seasonal allergic rhinitis (if onset of symptoms was anticipated within 4 weeks of randomization), perennial allergic rhinitis (if well controlled by regular use of INCS), or severe asthma. Additionally, patients were excluded if they exhibited a bilateral Zinreich score of less than 4 in 3 pairs of the posterior ethmoid, frontal and sphenoid sinuses (0-5 scale for each of sinuses) on screening MRI. Patients who were intolerant of topical anesthesia or corticosteroids, had received systemic corticosteroids (SCS) within 1 month prior to screening, or had a history or evidence of immunodeficiency, intracranial or orbital complication, evidence of mycetoma/fungal ball, sinus mucocele, or invasive fungal rhinosinusitis were also excluded.

Certain medications that could potentially interfere with study evaluations were not permitted from the time of the screening visit until the end of the study, except for their use as rescue treatment. Such rescue medications may include intranasal corticosteroids (INCS), oral/intramuscular corticosteroids (apart from a stable regimen of inhaled corticosteroids for asthma that had been taken for a minimum of 3 months prior to screening and would be maintained throughout the study), oral decongestants, and monoclonal antibodies. Anti-allergy medications were only allowed if patients continued such medication at a consistent dose from the screening visit through the study duration.

After screening assessment, patients underwent a 14-day minimum washout period. Patients were provided with and instructed to use nasal saline irrigations daily starting from the washout period to the end of the 24-week treatment period. Patients received no other active treatment for CRS during the washout period. Following the washout period, on the day of the procedure (Day 1), patients were randomized (1:1:1) to one of three study arms: bilateral administration of LYR-210 (7500 μg) into the middle meatus, bilateral administration of LYR-210 (2500 μg) into the middle meatus, or daily saline irrigation only control. To ensure that patients remained blinded to study treatment assignment, patients randomized to the control arm also received a sham procedure wherein the applicator was inserted into and withdrawn from the middle meatus. Patients received topical anesthetic and decongestant prior to the procedure and wore a blindfold to ensure blinding to treatment assignment. The clinical study investigator and clinical staff were unblinded to LYR-210 vs. control, however they were blinded to the LYR-210 dose administered. The sponsor was blinded to the study.

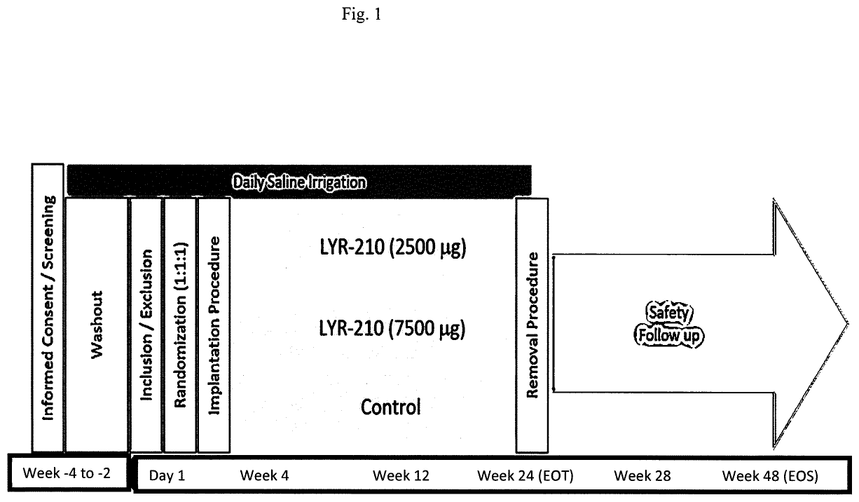

Patients returned to the clinic for follow-up assessments at weeks 4, 12 and 24, and had telephone follow-ups at weeks 8, 16, and 20 to record any adverse events (AEs) and concomitant medications/procedures. Patients wore an eye mask at all post-screening clinic visits only during endoscopies to remain blinded to the study treatment assignment. At the week 24 (end-of-treatment) visit, LYR-210 (2500 μg) or LYR-210 (7500 μg) were removed using standard instruments, and control patients underwent a sham-removal procedure. After the end-of-treatment visit, patients underwent a post-treatment follow up period lasting approximately 24 weeks. The LANTERN Phase 2 study design is summarized in FIG. 1.

### Safety Assessments

Safety was assessed by rates of AEs and changes in laboratory tests, vital signs, morning serum cortisol levels, nasal endoscopy assessment, intraocular pressure (TOP) and slit-lamp examination. AEs were coded using the Medical Dictionary for Regulatory Activities (MedDRA) dictionary Version 23.0. AEs were recorded throughout the study and reported for seriousness and relationship to study treatment or procedure. Severity of AEs was graded by the investigator as mild, moderate, or severe.

Ocular safety was assessed via measurement of TOP at baseline and weeks 4, 12, and 24, and dilated slit-lamp examination of the crystalline lens for presence of lens opacification at baseline and week 24. Lens opacity was graded using the WHO/PBD Simplified Cataract Grading System. Ocular assessments were conducted by ophthalmologists who were blinded to the study treatment received by the patient.

### Efficacy Assessments

Patient-reported symptoms and daily use of saline irrigation were captured using an electronic patient reported outcome (ePRO) system. Patients reported the score for each of the 4CS of CRS on the ePRO each morning, beginning at least 7 days prior to the Day 1 visit and continuing throughout the 24-week treatment period. Each of the four symptoms (nasal blockage, facial pain/pressure, nasal discharge, and loss of smell) was rated on a 0-3 scale (0=none, 1=mild, 2=moderate, and 3=severe). Patients also recorded the severity of their symptoms and social/emotional consequences of CRS via the SNOT-22¹⁴ at baseline and at weeks 2, 4, 8, 12, 16, 20, and 24. To evaluate the effect of LYR-210 in reducing sinonasal inflammation, patients underwent sinus MRI at baseline and end of treatment (week 24). The level of sinus opacification for each anterior and posterior ethmoid sinus was scored by an independent imaging core lab using the Zinreich (modified Lund-Mackay) scoring system which categorizes the percentage of opacification on a 0 to 5 scale (0=0%, 1=1-25%, 2=26-50%, 3=51-75%, 4=76-99%, and 5=100% or completely occluded).¹⁵ Time-to-first rescue treatment use was also evaluated and defined as worsening or acute exacerbation of CRS in an enrolled subject resulting in the investigator recommending INCS, SCS, decongestants, and/or FESS.

### Data Analysis

Patients who underwent an attempted or successful study treatment procedure were included in the safety analyses (the safety population). Efficacy analyses were conducted on the intention-to-treat (ITT) population which included Patients who underwent a successful study treatment procedure and had at least one post-randomization efficacy assessment. Patient demographics, medical history, and baseline disease characteristics were reported as frequencies or percentages of patients within the ITT patient population. Treatment emergent AEs were reported as counts of patients that experienced the event. For the primary and secondary endpoints of change from baseline (CFBL), an analysis of covariance (ANCOVA) model adjusting for treatment group, baseline score, and stratification variables (nasal allergy, nasal polyp) was used and tested at a one-sided significance level of 0.05.

No adjustments for multiplicity were made. The CFBL means were reported as least squares means (LSM) along with standard error (SE) from the ANCOVA model, unless otherwise noted. Time-to-first rescue treatment use was analyzed using the Kaplan-Meier method and hazard ratios. Subjects who did not achieve the event were censored at the end of treatment date or at the early termination date. Hazard ratios, two-sided 90% CIs and p-values were tested at a one-sided significance level of 0.05 are obtained from a Cox proportional hazards model. For symptom-based endpoints, if a patient dropped out of the trial or otherwise did not report data for a particular timepoint during the treatment period, the last observation carried forward (LOCF) approach was used to impute the missing values. Additionally, for patients that required a rescue treatment during the treatment period, the post-rescue data was set to missing and values for the post-rescue timepoints were imputed using LOCF. Statistical analyses were performed using SAS version 9.4 or higher.

Although various embodiments are specifically illustrated and described herein, it will be appreciated that modifications and variations of the present disclosure are covered by the above teachings and are within the purview of the appended claims without departing from the spirit and intended scope of the disclosure.

### ALTERNATIVE EMBODIMENTS

In one exemplary embodiment of the present implantable devices is a device comprising a permeable, non-permeable or semi-permeable sheet which contains an active ingredient. The sheet may be considered a permeable, non-permeable or semi-permeable membrane. The embodiment of the device comprising a sheet may contain osmotic drug delivery components as seen in FIG. 11. The permeable or semi-permeable sheets may be implanted flat or in a rolled state. In the rolled embodiment, the rolled sheet comprises an internal lumen. In this exemplary embodiment, drug delivery components are comprised of a semi-permeable polymeric hollow sheet containing a drug (e.g. MF) or other active pharmaceutical ingredient (API) in the absence or presence of an osmogen.

The implantable device may comprise a permeable, non-permeable or semi-permeable membrane, such as one or more fibers or a sheet. One embodiment, permeability to fluid is achieved through the use of permeable materials. In another embodiment, permeability is achieved through one or more delivery orifices on the hollow fiber or sheet wall. Any number of orifices is contemplated, including, but not limited to, one, two, three, four, five, six, seven, eight, nine, ten, twenty-five, fifty, one hundred, two hundred, a thousand, etc. A non-permeable embodiment is contemplated, for example, a metal tube with holes, wherein said holes may be drilled.

In one embodiment, the devices herein may be coated or covered. It is not intended for the present invention to be limited by the type, such as an elastomer, the thickness, or degree of coverage (e.g. partial or complete) of the coating. The device may be completely or partially coated. In one embodiment, there may be an elastomer coating on the top of the permeable or semi-permeable membrane, such as on the hollow fibers or sheet, covering or not covering any delivery orifices. Elastomers may be coated onto the implants to provide them with self-expandability. One or more orifices may be formed on the semi-permeable membrane either before or after the elastomer coating. Coatings may range, for example, from between about 1 μm to about 25 μm in thickness (e.g., ranging from about 1 to 2 to 5 to 20 to 25 vm in thickness), among other possibilities. Coating thicknesses may also be less than 1 μm or greater than 25 μm.

In one embodiment, the devices herein may be coated or covered. It is not intended for the present invention to be limited by the type, thickness, or coverage of the coating, such as an elastomer. The device may be completely or partially coated. In one embodiment, there may be an elastomer coating on the top of the permeable or semi-permeable membrane, such as the hollow fibers or sheet, covering or not covering any delivery orifices, as seen in FIG. 11. Elastomers may be coated onto the implants to provide them with self-expandability. One or more orifices may be formed on the semi-permeable membrane either before or after the elastomer coating.

1. A method of treating a sinus condition, comprising a) providing first and second implants, each comprising at least one coating containing about 7500 micrograms of mometasone furoate, and wherein the first implant is configured to fit inside a first middle meatus of a patient, and wherein the second implant is configured to fit inside a second middle meatus of said patient; b) implanting said first and second implants in a first and second middle meatus of a patient with symptoms of a sinus condition, and c) detecting a reduction in one or more symptoms, thereby treating said sinus condition.

2. The method of claim 1, wherein the mometasone furoate configured in each implant exhibits a zero-order release for 12 weeks or more of a planned implantation period.

3. The method of claim 1, wherein said reduction in one or more symptoms is reflected in a SNOT score.

4. The method of claim 1, wherein said sinus condition is a chronic sinus condition. The method of claim 1, wherein said sinus condition is characterized by at least two symptoms selected from the group consisting of nasal obstruction, nasal congestion, difficulty breathing through nasal passages, nasal polyp, nasal discharge, loss of smell, and facial pain.

6. The method of claim 1, wherein said sinus condition is characterized by two or more symptoms lasting at least 12 weeks selected from the group consisting of nasal discharge, nasal obstruction or congestion, hyposmia, and facial pressure or pain.

7. An implant configured to fit inside the middle meatus for preventing the need for surgery in a subject who is a candidate for sinus surgery, said implant comprising a coating comprising about 7500 micrograms of mometasone furoate, said implant configured to exhibit a zero-order release for at least 60% of said mometasone furoate, characterized in that the implant is delivered to the subject prior to surgery.

8. The implant of claim 7, wherein said implant is configured to release 20 to 80% of said mometasone furoate during the first 12 weeks.

9. The implant of claim 7, wherein said implant has a diameter of at least 13 mm.

10. The implant of claim 7, wherein said implant has a length of at least 10 mm.

11. A method of treating a sinus condition, a) implanting first and second implants in a first and second middle meatus of a patient with a sinus condition wherein said patient is a candidate for sinus surgery, each implant comprising at least one coating containing about 7500 micrograms of mometasone furoate, and b) detecting a reduction in one or more symptoms, wherein after 20 weeks said patient is no longer a candidate for sinus surgery, thereby treating said sinus condition.

12. The method of claim 11, wherein said patient is a candidate for sinus surgery based on a SNOT score of symptoms.

13. The method of claim 11, said patient is no longer a candidate for sinus surgery based on a SNOT score.

14. A combination for preventing the need for surgery in a subject who is a candidate for sinus surgery, comprising first and second implants, each comprising at least one coating containing about 7500 micrograms of mometasone furoate, wherein the first implant is configured to fit inside a first middle meatus of the subject, the mometasone furoate configured to have a zero-order release for more than 12 weeks; and wherein the second implant is configured to fit inside a second middle meatus of said subject, the mometasone furoate configured to have a zero-order release for more than 12 weeks, characterized in that the combination is delivered to the subject prior to surgery, wherein the subject is treated for at least 4 months.

15. The combination of claim 14, wherein said subject is a candidate for Functional Endoscopic Sinus surgery (FESS).

16. The combination of claim 14, wherein said first and second implants are configured to release 20 to 80% of said mometasone furoate during the first 12 weeks.

17. The combination of claim 14, wherein said each of said first and second implants is configured to exhibit a zero-order release after week 1.

18. A method of treating a sinus condition, comprising:
a) providing first and second implants, each comprising about 7500 micrograms of mometasone furoate;
b) implanting said first implant inside a first middle meatus of a first patient having a sinus condition;
c) implanting said second implant inside a second middle meatus of said first patient; and
d) monitoring the first patient's sinus condition for a period of at least 12 weeks, wherein the first patient implanted with first and second implants each comprising about 7500 micrograms of mometasone furoate exhibits a reduction in the need for rescue treatment when compared to a second patient having a sinus condition implanted with first and second implants each comprising about 2500 micrograms of mometasone furoate.

19. The method of claim 18, wherein said first and second implants are configured to release 20 to 80% of said mometasone furoate during the first 12 weeks.

20. The method of claim 18, wherein the sinus condition of said first patient improves more quickly than a second patient implanted with first and second implants comprising 2500 micrograms of mometasone furoate.

21. The method of claim 18, wherein said sinus condition is a chronic sinus condition.

22. The method of claim 18, wherein said monitoring of the first patient's sinus condition is done for a period of at least 16 weeks.

23. The method of claim 18, wherein said monitoring of the first patient's sinus condition is done for a period of at least 20 weeks.

24. The method of claim 18, wherein said first and second implants each comprise at least one coating, said coating containing about 7500 micrograms of mometasone furoate.

25. A method of treating a sinus condition, comprising:
a) providing first and second implants, each comprising about 7500 micrograms of mometasone furoate;
b) implanting said first implant inside a first middle meatus of a first patient having a sinus condition;
c) implanting said second implant inside a second middle meatus of said first patient; and
d) monitoring the first patient's sinus condition for a period of at least 12 weeks, wherein the first patient implanted with first and second implants each comprising about 7500 micrograms of mometasone furoate exhibits a reduction in the need for rescue treatment when compared to a second patient having a sinus condition given only saline irrigation treatment and no mometasone furoate.

26. The method of claim 25, wherein said first and second implants are configured to release 20 to 80% of said mometasone furoate during the first 12 weeks.

27. The method of claim 25, wherein the sinus condition of said first patient improves when compared to said second patient.

28. The method of claim 25, wherein said sinus condition is a chronic sinus condition.

29. A method of treating a sinus condition, comprising:
a) providing first and second implants, each comprising about 7500 micrograms of mometasone furoate;
b) implanting said first implant inside a first middle meatus of a first patient having a sinus condition;
c) implanting said second implant inside a second middle meatus of said first patient; and
d) monitoring the first patient's sinus condition for a period of at least 8 weeks, wherein the first patient implanted with first and second implants each comprising about 7500 micrograms of mometasone furoate improves more quickly than a second patient with a sinus condition implanted with first and second implants each comprising about 2500 micrograms of mometasone furoate.

30. The method of claim 29, wherein said improvement in the first patient implanted with first and second implants each comprising about 7500 micrograms of mometasone furoate, as compared to the second patient implanted with first and second implants each comprising about 2500 micrograms of mometasone furoate, is observed at least as early as 8 weeks after implantation of said implants.

31. The method of claim 29, wherein said first and second implants are configured to release 20 to 80% of said mometasone furoate during the first 12 weeks.

32. The method of claim 29, wherein said sinus condition is a chronic sinus condition.

33. The method of claim 29, wherein said first patient is a patient with nasal polyps.

34. The method of claim 29, wherein said 7500 μg implants reduced rescue treatment use for said first patient.

35. The method of claim 29, wherein said 7500 μg implants reduced radiographic ethmoid opacification at week 24 in said first patient.

36. The method of claim 29, further comprising e) measuring plasma concentrations of mometasone furoate in said first patient.

37. The method of claim 36, wherein said patient displayed dose-dependent plasma concentrations.

38. A method of treating a sinus condition, comprising:
a) providing first and second implants, each comprising mometasone furoate;
b) implanting said first implant inside a first middle meatus of a patient having a sinus condition;
c) implanting said second implant inside a second middle meatus of said patient;
d) removing said first and second implants from said patient; and
e) detecting improvement in said patient's sinus condition after said implants are removed for a period of at least 4 weeks.

39. The method of claim 38, wherein the patient is implanted with first and second implants each comprising about 7500 micrograms of mometasone furoate.

40. The method of claim 38, wherein the patient is implanted with first and second implants each comprising about 2500 micrograms of mometasone furoate.

41. The method of claim 38, wherein said improvement detected in said patient's sinus condition is reduced nasal blockage, facial pain, nasal discharge (anterior/posterior), and/or loss of smell.

42. The method of claim 38, wherein said patient's condition continues to improve for a period of at least 8 weeks after the implants are removed.

43. The method of claim 38, wherein said patient's condition continues to improve for a period of at least 12 weeks after the implants are removed.

44. The method of claim 38, wherein said patient's condition continues to improve for a period of at least 16 weeks after the implants are removed.

45. The method of claim 38, wherein said implants are removed after 24 weeks.
